(19) United States
(12) Patent Application Publication
(10) Pub. No.: US 2023/0000888 A1
(43) Pub. Date: Jan. 5, 2023

(54) NAD-PRECURSORS AND DIETARY RESTRICTION FOR TREATING AGE RELATED MEDICAL CONDITIONS

(71) Applicant: Leibniz-Institut für Alternsforschung - Fritz-Lipmann-Institut E.V. (FLI), Jena (DE)

(72) Inventors: Karl Lenhard Rudolph, Jena (DE); Elias Amro, Jena (DE); Yulin Chen, Jena (DE)

(73) Assignee: Leibniz-Institut für Alternsforschung - Fritz-Lipmann-Institut E.V. (FLI), Jena (DE)

(21) Appl. No.: 17/782,251

(22) PCT Filed: Dec. 18, 2020
(86) PCT No.: PCT/EP2020/087135

§ 371 Date: Jun. 3, 2022

(30) Foreign Application Priority Data
Dec. 19, 2019 (EP) 19218069.3

(51) Int. Cl. A61K 31/706; A61K 31/05; A61K 31/155; A61K 31/194; A61K 31/454; A61K 31/385; A61K 31/7004; A61K 31/436; A61P 43/00

(52) U.S. Cl. CPC A61K 31/706; A61K 31/05; A61K 31/155; A61K 31/194; A61K 31/454; A61K 31/385; A61K 31/7004; A61K 31/436; A61P 43/00

(57) ABSTRACT

A nicotinamide adenine dinucleotide (NAD) precursor is provided for use in the treatment and/or prevention of an age-related medical condition in a subject. The NAD precursor is administered in combination with a calorie restriction diet (CRD) and/or a calorie restriction mimetic (CRM). Furthermore, a pharmaceutical combination is provided that includes a NAD precursor and a CRM.

The invention relates to the field of medical treatment of age-related disease and the prevention or slowing of aging. The invention therefore relates to a nicotinamide adenine dinucleotide (NAD) precursor for use in the treatment and/or prevention of an age-related medical condition in a subject, wherein said NAD precursor is administered in combination with a calorie restriction diet (CRD) and/or a calorie restriction mimetic (CRM). The invention further relates to a pharmaceutical combination comprising a NAD precursor and a CRM.

### BACKGROUND OF THE INVENTION

Moderate reduction in dietary food intake (also referred to dietary restriction/DR or calorie restriction diet/CRD) is the best proven intervention to increase lifespan across species, including yeast (Wei et al., 2008), Drosophila melanogaster (Grandison et al., 2009; Lee et al., 2008), Caenorhabditis elegans (C. elegans) (Fontana et al., 2010) and some strains of laboratory mice (Mitchell et al., 2016). Mechanistically, it has been shown that reduction in nutrient availability in response to DR induces changes in various signaling pathways that can slow down the aging process and contribute to increases in longevity in model organisms (Collino et al., 2013). The most prominent pathways contributing to lifespan extension in response to DR include (i) the suppression of growth promoting pathways (Kapahi et al., 2004), (ii) the induction of metabolic fitness and mitochondrial function (Zid et al., 2009), and (iii) the activation of repair pathways (Vermeij et al., 2016).

The positive effects of DR on lifespan and/or health-parameters in various species have fueled discussions about the potential use of DR or DR mimicking drugs (such as metformin) to improve health in elderly humans (Barzilai et al., 2016). However, since most of the current knowledge comes from studies where these interventions have been started during early adulthood, little is known about the consequences of such approaches when applied late in life in older individuals. Of note, several key pathways (such as IGF and GH signaling) that are downregulated by DR and contribute to the health and lifespan extending effect of DR, are also downregulated during normal aging and contribute to the development of tissue atrophy (Carter et al., 2002). There is experimental evidence that DR-induced changes in signaling pathways that also occur during normal aging, can negatively impact on aging in model organisms (Benedetto and Gems, 2019; Wilhelm et al., 2017) and in genetic, progeroid mouse models of accelerated aging (Marino et al., 2010).

Together these data suggest that the beneficial effects of DR on health and lifespan extension may depend on the timing of the intervention and could have different outcomes when started in early adulthood vs. later in life. Only a few studies have started to look into this question. In the fruit fly, Drosophila melanogaster, it was found that DR decreases mortality rates within 2 days after beginning the intervention, independent of the age of the fly when the diet was initiated (Mair et al., 2003). In mice, DR has the potential to reduce cancer incidence and to elongate lifespan when started in middle aged, 12-month old mice (Volk et al., 1994). However, when DR was applied to middle aged mice it only prevented a small fraction (19%) of transcriptome changes that occur during aging of ad libitum fed mice (Lee et al., 2002), whereas when applied to young mice, DR appeared to have a higher potential in preventing age-related changes in gene expression; 29% were completely prevented and 34% partially (Lee et al., 1999). This interpretation is supported by a study showing that DR has a reduced potential to revert aging-associated changes in gene expression in white adipose tissue and to decrease mortality rates, when applied to old mice compared to young mice (Hahn et al., 2019).

It has been postulated that increases in mitochondrial function in response to decreases in nutrient availability by itself contribute to the activation of stress responses that can lead to increases in cellular and organism fitness and lifespan extension (Guarente, 2008). Different mechanisms have been implicated to mediate mitochondria-induced pro-longevity responses, including shifts in NAD+/NADH ratios, which can lead to induction of Sirtuin-signaling (Satoh et al., 2013) or the induction of ROS-dependent hormesis responses (Lee et al., 2010; Zarse et al., 2012). Compositions that affect the concentration of NAD, including for example NAD precursors such as nicotinamide riboside (NR), have been suggested for mitigation of the effects of aging and for improving cell and tissue health, for example of the liver (WO2019/175587A1, WO2017/184885A1, WO2018/170389A1). Furthermore, Dellinger et al. (“Repeat dose NRPT (nicotinamide riboside and pterostilbene) increases NAD+ levels in humans safely and sustainably: a randomized, double-blind, placebo-controlled study”, NPJ Aging and Mechanisms of Disease, vol. 3, no. 1, 24 Nov. 2017) describe that administration of NR in combination with pterostilbene (a SIRT1 activator) is well tolerated in elderly patients and it was speculated that this combination may have positive effects on blood pressure and liver health, which however, was only a hypothetical thought, not supported by any of the measurements in the publication.

While experimental evidence supports the concept that mitochondrial responses are an integral part of the metabolic response to changes in nutrient availability (Ito et al., 2012; Snoeck, 2017), it is currently unclear how aging related loss of mitochondrial function may interfere with such responses. Of note, aging-associated mitochondrial dysfunction has been documented across various tissues (Akbari et al., 2019; Balaban et al., 2005; Green et al., 2011; Haas, 2019) and was also demonstrated to impair the functionality of hematopoietic stem cells (HSCs) in aging mice (Ho et al., 2017).

Together, it is currently unclear whether DR has the same potential (which is seen in response to DR treatment started young animals) to improve metabolic fitness, health parameters, and cellular function, when applied late in life to aged animals or humans. Given the current discussion on the use of DR and DR-mimetics for the prevention of age-associated diseases in humans, functional studies on this question are of great importance.

In summary, it has been shown that continuous application/administration of DR and DR-mimetics to young and middle-aged individuals have a positive effect on age-associated functional decreases, such as decreasing cellular function and decreasing organ function, which are associated with age-related medical conditions. DR and DR-mimetics can slow down or prevent such functional decreases. However, recent results indicate that DR and DR-mimetics lose this activity in individuals and cells or such individuals that have passed a certain age, which may be referred to as old individuals. It is unclear, whether such old individuals and/or cells of such old individuals have lost the potential to respond to DR and DR-mimetics, or whether the potential to respond to DR and DR-mimetics can be reactivated in such cells.

In light of the prior art there remains a significant need to provide additional means for enabling treatment and/or prevention of age-related medical condition in a subject that have passed a certain age, such as in old individuals. In particular, there is a need to provide means for making the beneficial effect of DR and DR-mimetics available to old individuals.

### SUMMARY OF THE INVENTION

In light of the prior art the technical problem underlying the present invention is to provide improved or alternative means for enabling the beneficial effects of DR and DR-mimetics on life span and age-related decreases in cell function and/or organ function, preferably in old or aging subjects.

This problem is solved by the features of the independent claims. Preferred embodiments of the present invention are provided by the dependent claims.

The invention therefore relates to a nicotinamide adenine dinucleotide (NAD) precursor for use in the treatment and/or prevention of an age-related medical condition in a subject, wherein said NAD precursor is administered in combination with a calorie restriction diet (CRD) and/or a calorie restriction mimetic (CRM).

The present invention is based on the entirely surprising finding that aging-associated mitochondrial dysfunction abrogates the metabolic plasticity of cells and individuals to respond to changes in nutrient availability, which mediate the beneficial effects of CRD and CRM.

Herein, the terms “reduction in dietary food intake”, “dietary restriction (DR)” and “calorie restriction diet (CRD)” are used synonymously.

As shown in the examples below, young cells and individuals can adapt to CRD-induced reduction in nutrient availability by activation of mitochondrial metabolism, metabolic stress responses, and/or mitophagy, resulting in improved cellular function, also in vivo. In contrast, old individuals and cells isolated from old individuals fail to enhance mitochondrial function or to activate stress response pathways in response to CRD. Consequently, older individuals typically do not profit form CRD, if CRD is initiated when the individuals have already reached an age considered “old” in the sense of the present invention.

However, it was surprisingly found that the improvement of mitochondrial function by administration of an NAD precursor to old cells and/or old individuals can restore metabolic plasticity in response to CRD, which makes the beneficial effects of CRD and/or CRM available when administration is started in old individuals.

Old individuals lose the potential to respond to CRM and/or CRD, i.e. by undergoing improvements in cellular functions that would lead to an enhancement of organ function and an extension of lifespan of individuals which are constantly exposed to CRM and/CRD from a young age onwards. In contrast to young individuals and their cells, old individuals and their cells have lost the potential to metabolically adapt to CRM and/or CRD, which can be reflected in a stabilization of body weight in response to reduced nutrient availability. This loss in metabolic plasticity in aging individuals is associated with age-related mitochondrial dysfunction. This results in defects in nutrient sensing and defect in the activation of cellular stress response pathways in old individuals and cells of old individuals. However, nutrient sensing and the activation of cellular stress response pathways have been shown to be among the major mechanisms mediating beneficial effects of CRM and/or CRD on disease prevention and lifespan extension.

Surprisingly, it was found that CRD/CRM has the capacity to significantly ameliorate some differences between cells from old and young individuals. However, age-related alterations of NAD dependent biological processes, in particular sirtuin-mediated signaling pathways and proteins indicative of mitochondrial (dys)function, remain unchanged in old cells under administration CRD/CRM. Important mitochondrial enzymes that need NAD or its derivate NADH include enzymes of the “tricarboxylic acid cycle” and the “electron transport chain”. The activation of these enzymes leads to a strong increase of oxygene consumption of the young cells, but a failure of the old cells to induce this activity, which can however, fully be rescued by NAD-supplementation. Due to this entirely unexpected finding, it was tested whether provision of an NAD precursor could enable the regulation of NAD dependent biological processes in response to CRD/CRM in cells from old individuals, which was indeed the case.

Accordingly, the combined administration of an NAD precursor and CRM and/or CRD enables the treatment and/or prevention of an age-related medical condition in a subject, also when the subject is an old subject.

In summary, the present invention provides means for treating or preventing the decline in cell function and organ maintenance, which occurs with age. It was known already that CRM and/or CRD can counteract this, for example by inhibiting the age-associated loss of stem cell function and prolonging the life span, and CRM and/or CRD is the best-known intervention today that can achieve an extension of health and life span in different species. However, it was found recently that CRM and/or CRD loses its efficacy when applied in old age. As such, when administration of CRM and/or CRD is initiated in old individuals it is most likely too late and CRM and/or CRD can only marginally affect mortality and the function of cells and in particular stem cells cannot be improved by CRM and/or CRD in old age (Hahn et al. 2019).

The present invention provides a surprising solution for this problem, as a decrease in mitochondrial function was identified as the cause of the loss of efficacy of CRM and/or CRD in old age. Mitochondrial function is required for metabolic changes and stress responses that are activated by CRM and/or CRD and that lead to a strengthening of stem cell function in young/medium age in response to a reduced diet. However, due to the loss of mitochondrial function, these metabolic changes and stress responses cannot occur in old subjects, which would receive CRM and/or CRD.

In embodiments of the invention, the NAD precursor is nicotinamide riboside (NR). It has not been described until now in the art that administration of an NAD precursor, such as NR, can restore the ability of aged cells and in particular stem cells to activate stress signals in response to CRM and/or CRD by improving mitochondrial function. This completely unexpected finding is one aspect that led to the present invention.

As shown herein, NAD precursors can restore the efficacy of CRM and/or CRD in old individuals and cells of such individuals, leading in particular to an improved stem cell function and overall health, and to an extended lifespan. The examples provided herein demonstrate that the claimed combination of CRM and/or CRD and an NAD precursor is particularly promising to improve stem cell function and health, also if administration/application is only started at a relatively old age.

In preferred embodiments of the invention, the CRM is a non-chemical CRM, such as preferably a fasting mimicking diet product, bariatric surgery or exercise. It is particularly preferred in the context of the invention to combine administration of an NAD precursor with a meal kit or a prepackaged meal providing an CRD or mimicking the effects of a CRD. In the context of the invention, such meal kits or prepackaged meals can be regarded as a CRD or a non-chemical CRM. It was entirely unexpected that a CRD or a non-chemical CRM can achieve a synergistic effect when administered/provided in combination with an NAD precursor. As shown herein, administration of an NAD precursor restores the ability of an older individual, such as a human subject that is over 40 years old to benefit and profit from a CRD or non-chemical CRM only when the intervention is combined with NAD precursor supplementation. Only this combined application is lifespan extending. The necessity of combining these two interventions (CRD/CRM plus NAD precursor application) to achieve health benefits in older individuals was completely unknown.

In embodiments of the invention, the CRM is a fasting mimicking diet product. The use of fasting mimicking diet products can be beneficial in the context of the present invention, since CRD may be difficult to maintain for long periods, in particular in humans from western countries, and therefore a CRM product is often associated with an improved compliance of the subject in comparison to a CRD.

Instead, by using a fasting mimicking diet product, such as a kit providing specific food and beverages, such as a (therapeutic) meal package for use in providing meals to a subject, it is more likely that the subject continuously maintains the treatment regime. Such packages for providing foods and beverages in a prepacked form in order to provide a fasting mimicking diet are known in the art, such as the products described in WO 2014/127000 and WO 2009/132320, their content, and that of their US counterparts, is incorporated by reference.

In further embodiments, the CRM is a chemical compound/drug, preferably selected from the group consisting of resveratrol, metformin, oxaloacetate, rimonabant, lipoic acid, 2-deoxy-D-glucose and rapamycin or other TOR (Target of Rapamycin)—inhibitors. The use of compounds or drugs that function as CRM is advantageous in the context of the present invention, since the subject may not have to change its diet habit at all but instead can be treated with an NAD precursor and a CRM compound/drug in order to achieve the effect of the present invention.

In preferred embodiments of the invention, the NAD-precursor is administered in combination with a CRD, a non-chemical CRM and/or a mitochondria stress-inducing CRM.

In preferred embodiments of the invention, the NAD-precursor is administered in combination with a CRD and/or a mitochondria stress-inducing CRM.

In preferred embodiments of the invention, the NAD-precursor is administered in combination a mitochondria stress-inducing CRM.

Preferably, the mitochondria stress-inducing CRM is a CRM that mainly acts through AMPK activation. Preferably, the mitochondria stress-inducing CRM is mediating mitochondria stress, for example inhibition of mitochondrial complexes, for example complex I, through AMPK, whereas the effect of such CRMs on Sirtuin signaling or induction of autophagy is rather indirect. Examples of mitochondria stress-inducing CRM include metformin, 2-deoxy-D-glucose, oxaloacetate and rimonabant. Preferred examples include metformin, 2-deoxy-D-glucose, oxaloacetate, wherein metformin is particularly preferred. Surprisingly, it could be shown that NAD precursor supplementation in combination with the subclass of mitochondria stress-inducing CRMs has strong synergistic effects on the induction of mitochondrial activity, which in turn leads to the induction of stress signals that improve health in aging. This is a surprising advantage of these specific CRMs, that can also be observed in response to CRD. It appears that activation of Sirtuin signaling or induction of autophagy by chemical compounds does not induce this synergistic effect.

CRM and/or CRD activate metabolic activity and stress responses in cells isolated from young individuals, such as hematopoietic stem cells, leading to an improved hematopoiesis. However, this ability to respond to CRM/CRD is lost with age. However, it was unexpectedly found that impaired ability to respond to CRM/CRD in aged individuals is due to a loss in mitochondria function and that the reactivation of mitochondrial function by administration of an NAD precursor, is sufficient to restore the ability of cells of old individuals to respond to CRM and/or CRD.

In embodiments of the invention, the age-related medical condition is an aging-associated disease. In further embodiments, the age-related medical condition is an aging-associated dysfunction. In embodiments of the invention, the age-related medical condition, which may be an aging-associated disease or dysfunction, is associated with a decline in mitochondrial function.

In embodiments, the age-related medical condition associated with a decline in mitochondrial function is selected from the group comprising or consisting of myocardial dysfunction, myocardial infarction, heart failure, liver failure, nonalcoholic fatty liver disease (NAFLD), nonalcoholic steatohepatitis (NASH), chronic kidney disease, acute kidney injury, kidney failure, muscle atrophy, sarcopenia, cardiomyopathy, cardiovascular disease, cancer, diabetes, metabolic syndrome, neuropathies, neurodegenerative disorders such as amyotrophic lateral sclerosis (ALS), multiple sclerosis, Parkinson's disease, and Alzheimer's disease.

In embodiments, the treatment and/or prevention of an age-related medical condition comprises slowing, reversing and/or inhibiting the ageing process.

In preferred embodiments, the subject is human. In further embodiments, the subject is human and is more than 40 years old.

In embodiments of the invention, the NAD precursor antagonizes an age-related decline of mitochondrial function, preferably wherein the NAD precursor enhances and/or restores a therapeutic response of a human subject, more preferably a subject over 40 years old, to CRD and/or CRM administration.

The present invention is particularly suited for old and middle-aged human subjects, such as human subjects over 40 years old, since the metabolic response CRM and/or CRD is impaired with age and can even completely disappear. However, administration of an NAD precursor reactivates mitochondrial functions that decline with age and make old and middle-aged individuals susceptible to the benefits of CRM and/or CRD.

The invention further relates to a NAD precursor for use in combination with CRD and/or CRM as disclosed herein, wherein the CRD and/or CRM promote metabolic plasticity, mitochondrial metabolism and/or metabolic stress responses. It is a particular advantage of the present invention that CRD and/or CRM act by promoting metabolic plasticity, mitochondrial metabolism and/or metabolic stress responses, which are enhanced, promoted and/or reactivated by NAD precursors, in particular in old individuals.

The present invention further relates to a pharmaceutical combination, comprising
a. a NAD precursor, and
b. a CRM.

The pharmaceutical combination of the present invention is particularly advantageous, since the two comprised components, namely the NAD precursor and the CRM act synergistically in the treatment and/or prevention of an age-related medical condition. This is in particular the case if administration or treatment of the pharmaceutical composition is only started in a subject that can be considered an old subject, as defined herein.

Old individuals have often lost the metabolic plasticity and mitochondrial function required for profiting from a CRD and/or CRM, but the NAD precursor reactivates declined mitochondrial function enabling metabolic plasticity so that also old individual can profit from the beneficial effects of the CRM.

In embodiments of the pharmaceutical combination of the invention the CRM is selected from the group comprising or consisting of resveratrol, metformin, oxaloacetate, rimonabant, lipoic acid, 2-deoxy-D-glucose and rapamycin (or other TOR-inhibitors) or a fasting mimicking diet product. Preferably, the CRM is a fasting mimicking diet product, such as a meal kit or a meal package providing a CRD or composed to mimic the effects of a CRD.

In embodiments of the pharmaceutical combination of the invention the NAD precursor is in a pharmaceutical composition in admixture with a pharmaceutically acceptable carrier, and the CRM compound is in a separate pharmaceutical composition in admixture with a pharmaceutically acceptable carrier.

In further embodiments of the pharmaceutical combination of the invention the NAD precursor and the CRM compound are present in a kit, in spatial proximity but in separate containers and/or compositions.

In some embodiments, the invention relates to a kit comprising an NAD precursor and a fasting mimicking diet product (preferably a prepackaged meal or meal kit providing the effect of a CRD), e.g. in spatial proximity but in separate containers and/or compositions or combined in the diet product. Packages for providing foods and beverages in a prepacked form in order to provide a fasting mimicking diet are known in the art (WO 2014/127000 and WO 2009/132320), whereby the NAD precursor may be combined or added to said packages.

Furthermore, in the context of the pharmaceutical combination of the invention, in some embodiments the NAD precursor and the CRM compound may be combined in a single pharmaceutical composition in admixture with a pharmaceutically acceptable carrier.

The present invention also relates to pharmaceutical combination as described herein for use in the treatment and/or prevention of an age-related medical condition.

In embodiments, the invention relates to pharmaceutical combination as described herein for use in the treatment and/or prevention of an age-related medical condition, wherein the age-related medical condition is associated with a decline in mitochondrial function.

All features disclosed in the context of the NAD precursor of the invention for use in the treatment and/or prevention of an age-related medical condition in a subject are herewith also disclosed in the context of the pharmaceutical composition of the invention, and the pharmaceutical composition of the invention for use in the treatment and/or prevention of an age-related medical condition, and vice versa.

### DETAILED DESCRIPTION OF THE INVENTION

All cited documents of the patent and non-patent literature are hereby incorporated by reference in their entirety.

The invention relates to a nicotinamide adenine dinucleotide (NAD) precursor for use in the treatment and/or prevention of an age-related medical condition in a subject, wherein said NAD precursor is administered in combination with a calorie restriction diet (CRD) and/or a calorie restriction mimetic (CRM).

NAD and NAD Precursors

Nicotinamide adenine dinucleotide (NAD) is a cofactor that is central to metabolism. NAD is found in all living cells and is called a dinucleotide because it consists of two nucleotides joined through their phosphate groups. One nucleotide contains an adenine nucleobase and the other nicotinamide. NAD exists in two forms: an oxidized and reduced form, abbreviated as NAD+ and NADH respectively. In metabolism, NAD is involved in redox reactions, carrying electrons from one reaction to another. The cofactor is, therefore, found in two forms in cells: NAD+ is an oxidizing agent—it accepts electrons from other molecules and becomes reduced. This reaction forms NADH, which can then be used as a reducing agent to donate electrons. These electron transfer reactions are the main function of NAD. However, it is also used in other cellular processes, most notably a substrate of enzymes that add or remove chemical groups from proteins, in posttranslational modifications. Because of the importance of these functions, the enzymes involved in NAD metabolism are targets for drug discovery. NAD+ is synthesized through two metabolic pathways. It is produced either in a de novo pathway from amino acids or in salvage pathways by recycling preformed components such as nicotinamide back to NAD+.

As used herein, the term “NAD precursor” comprises, without limitation, nicotinic acid (NA), nicotinamide riboside (NR), nicotinamide mononucleotide (NMN) and nicotinamide (Nam), and derivatives thereof.

Eukaryotes can synthesize NAD+de novo via the kynurenine pathway from tryptophan (Krehl, et al. Science (1945) 101:489-490; Schutz and Feigelson, J Biol. Chem. (1972) 247:5327-5332) and niacin supplementation prevents the pellagra that can occur in populations with a tryptophan-poor diet. It is well-established that nicotinic acid is phosphoribosylated to nicotinic acid mononucleotide (NaMN), which is then adenylylated to form nicotinic acid adenine dinucleotide (NaAD), which in turn is amidated to form NAD+(Preiss and Handler, J Biol. Chem. (1958) 233:488-492; Ibid., 493-50). Nicotinamide Adenine Dinucleotide (“NAD+”) is an enzyme co-factor that is essential for the function of several enzymes related to reduction-oxidation reactions and energy metabolism. (Katrina L. Bogan & Charles Brenner, Nicotinic Acid, Nicotinamide, ami Nicotinamide Riboside: A Molecular Evaluation of NAD+ Precursor Vitamins in Nutritions, 28 Annual Review of Nutrition 115 (2008)). NAD functions as an electron carrier in cell metabolism of amino acids, fatty acids, and carbohydrates. (Bogan & Brenner 2008). NAD⁺ serves as an activator and substrate for sirtuins, a family of protein deacetylases that have been implicated in metabolic function and extended lifespan in lower organisms, (Laurent Mouchiroud et al., The NAD⁺/Sirtuin Pathway Modulates Longevity through Activation of Mitochondrial UPR and FOXO Signaling, 154 Cell 430 (2013)). The co-enzymatic activity of NAD⁺, together with the tight regulation of its biosynthesis and bioavailability, makes it an important metabolic monitoring system that is clearly involved in the aging process. Once converted intracellularly to NAD(P)⁺, vitamin B3 is used as a co-substrate in two types of intracellular modifications, which control numerous essential signaling events (adenosine diphosphate ribosylation and deacetylation), and is a cofactor for over 400 reduction-oxidation enzymes, thus controlling metabolism. This is demonstrated by a range of metabolic endpoints including the deacetylation of key regulatory proteins, increased mitochondrial activity, and oxygen consumption. Critically, the NAD(P)(H)-cofactor family can promote mitochondrial dysfunction and cellular impairment if present in sub-optimal intracellular concentrations. Vitamin B3 deficiency yields to evidenced compromised cellular activity through NAD⁺ depletion, and the beneficial effect of additional NAD⁺ bioavailability through nicotinic acid (“NA”), nicotinamide (“Nam”), and nicotinamide riboside (“NR”) supplementation is primarily observed in cells and tissues where metabolism and mitochondrial function had been compromised.

The bulk of intracellular NAD⁺ is believed to be regenerated via the effective salvage of nicotinamide (“Nam”) while de novo NAD⁺ is obtained from tryptophan. (Anthony Rongvaux et al., Reconstructing eukaryotic NAD metabolism, 25 BioEssays 683 (2003)). Crucially, these salvage and de novo pathways apparently depend on the functional forms of vitamins BI, B2, and B6 to generate NAD⁺ via a phosphoriboside pyrophosphate intermediate. Nicotinamide riboside (“NR”) is the only form of vitamin B3 from which NAD⁺ can be generated in a manner independent of vitamins BI, B2, and B6, and the salvage pathway using nicotinamide riboside (“NR.”) for the production of NAD is expressed in most eukaryotes. The main NAD+ precursors that feed the salvage pathways are Nam and NR. (Bogan & Brenner 2008). Studies have shown that NR is used in a conserved salvage pathway that leads to NAD⁺ synthesis through the formation of nicotinamide mononucleotide (“NMN”). Upon entry into the cell, NR is phosphorylated by the NR kinases (“NRKs”), generating NMN, which is then coverted to NAD+ by nicotinamide mononucleotide adenylyltransferase (“NMN AT”), (Bogan & Brenner 2008). Because NMN is the only metabolite that can be converted to NAD in mitochondria, nicotinamide (“Nam”) and nicotinamide riboside (“NR”) are the two candidate NAD+ precursors that can replenish NAD+ and thus improve mitochondrial fuel oxidation. A key difference is that nicotinamide riboside (“NR”) has a direct two-step pathway to NAD synthesis that bypasses the rate-limiting step of the salvage pathway, nicotinamide phosphoribosyltransferase (“NAMPT”). Nicotinamide (“Nam”) requires NAMPT activity to produce NAD+. This reinforces the fact that nicotinamide riboside (“NR”) is a very effective NAD precursor, Conversely, deficiency in dietary NAD precursors and/or tryptophan causes pellagra, a disease characterized by dermatitis, diarrhea, and dementia. (Bogan & Brenner 2008), In summary, NAD+ is required for normal mitochondrial function, and because mitochondria are the powerhouses of the cell, NAD+ is required for energy production within cells.

Nicotinamide riboside (“NR”) is a pyridinium compound having the formula (I):

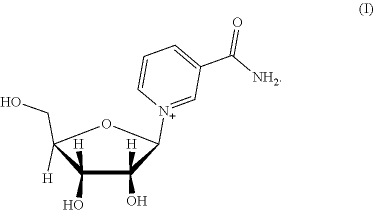

Nicotinamide riboside (“NR”) is available in a reduced form (“NRH”) as a 1,4- dihydropyridine compound having the formula (I-H):

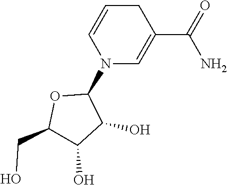 [SMILES: NC(=O)C1=CN([C@@H]2O[C@H](CO)[C@@H](O)[C@H]2O)C=CC1]

NAD precursors of the invention in particular comprise compounds of the formula (Ia):

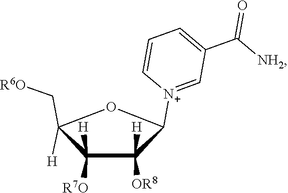

wherein R⁶ is selected from the group consisting of hydrogen, —C(O)R′, —C(O)OR′, —C(O)NHR′, substituted or unsubstituted C₈)alkyl, substituted or unsubstituted C₈)cycloalkyl, substituted or unsubstituted aryl, substituted or unsubstituted heteroaryl, and substituted or unsubstituted heterocycle; R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl; and R⁷ and R⁸ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₈)alkyl.

Further embodiments of NAD precursors are described in WO2017/184885A1 and can be identified on the basis of the description provided herein by a person skilled in the art. Further NAD precursors of the present invention are disclosed in WO2018/170389A1.

In addition, to the supplementation of NAD-precursors, chemical compounds that increase the production of NAD from intrinsic precursors, such as tryptophan, can also be employed to be combined with CRD or CRMs. Specifically, inhibition of the enzyme “α-amino-β-carboxymuconate-ϵ-semialdehyde decarboxylase (ACMSD)” leads to enhanced production of NAD from tryptophan degradation within the kynurenine pathway (Katsyuba et al. 2018, “De novo NAD+ synthesis enhances mitochondrial function and improves health” Nature 2018 November; 563(7731):354-359). In the context of the present invention it is understood that ACMSD inhibitors qualify as NAD-precursors. Based on our invention a combination of ACMSD inhibition with CRMs or CRDs would have the same beneficial effects as combining NAD-precursor supplementation with CRDs or CRMs and is therefore comprised by the scope of the present invention. Accordingly, in embodiments, the NAD-precursor is an ACMSD inhibitor.

Calorie Restriction Diet (CRD) and Calorie Restriction Mimetics (CRM)

The term “calorie restriction diet” (CRD), which is used synonymously with the term dietary restriction (DR) or dietary restriction diet in the context of the present invention, relates to a dietary regimen that reduces food intake without incurring malnutrition and while maintaining adequate nutrition. “Reduce” can be defined relative to the subject's previous intake before intentionally restricting food or beverage consumption, or relative to an average person of similar body type. Calorie restriction is typically adopted intentionally to reduce body weight. In embodiments, CRD relates to a diet that reduces the intake of energy from food and beverages via the diet by 10-50%, preferably 15-45, 20-40, 25-35 or about 30% compared to an ad libitum diet. In embodiments, the term refers to a diet that reduces the intake of energy from food and beverages via the diet by 10-50%, preferably 15-45, 20-40, 25-35 or about 30% compared to an average person of similar body type. In further embodiments, CRD relates to a diet that reduces the intake of energy from food and beverages via the diet by 10-50%, preferably 15-45, 20-40, 25-35 or about 30% compared to recommended dietary guidelines, such as those released by the US Department of Health and Human Services or the Guidelines of the ACC/AHA (“2019 ACC/AHA Guideline on the Primary Prevention of Cardiovascular Disease”. Circulation. 140 (11): e596-e646).

In embodiments, a CRD only restricts certain aspects or components of a usual diet, wherein the amounts of calories are not necessarily reduced in such a CRD. Accordingly, CRD comprises dietary regimes that reduce or restrict the consumption of certain components of a standard diet. This includes a reduced provision of calories, but may also relate to a reduction of, for example, carbohydrates, in particular food sugar, proteins, specific amino-acids (such as metformin), or fat, which may be associated with a reduction of calories or not.

CRD preferably refers to a reduction of the average daily caloric intake. CRD has been shown to prolong the lifespan of experimental animal models such as nematodes, flies, and mice and delays the progression of diverse age-related changes, dysfunctions and diseases. CR not only extends the lifespan of a wide range of animals, but also protects against many aging-related diseases such as type 2 diabetes, cardiovascular diseases, cancer and neurodegeneration in rodents. More importantly, studies with rhesus monkeys indicate that long-term CR also decreases aging-related mortality and diseases in non-human primates. Cellular pathways and mechanism that are affected by CRD include AMPK, mTOR, Sirtuins and ROS and systems involved in the redox balance of cells.

As used herein, the term calorie restriction mimetic (CRM) relates to methods, products or compounds that reproduce the effect of CRD without limiting the amount of food. As used herein, the term calorie restriction mimetic (CRM) may also be termed dietary restriction mimetic. CRMs exhibit the systemic effects of CRD and may include not only drugs or chemical compounds but also methods such as bariatric surgery or exercise and products, such as fasting mimicking diet products. Accordingly, the term CRM comprises different categories of CRMs, in particular CRMs that are based on chemical compounds or drugs on the one hand, and non-chemical CRMs including fasting mimicking diet products in form of kits or prepacked meal packages and further measures such as bariatric surgery and exercise on the other hand. It is evident that fasting mimicking diet products may also be classified as a CRD, since such meal packages provide a specific diet.

Furthermore, CRMs of the invention, and in particular CRMs based on chemical compounds or drugs, comprise downstream and upstream CRMs. Various CRM are known in the art and a skilled person is able to identify CRMs for use in the context of the present invention, as review by Shintani et al (Nutrients. 2018 December; 10(12): 1821).

As used herein, the term calorie restriction mimetics (CRM), also known as energy restriction mimetics, can refer to a hypothetical class of dietary supplements or drug candidates that would, in principle, mimic the substantial anti-aging effects that calorie restriction diet (CRD) has on many laboratory animals and humans. CRD may be defined as a reduction in calorie intake of 20% (mild CR) to 50% (severe CR) without incurring malnutrition or a reduction in essential nutrients. An effective CRM can alter the key metabolic pathways involved in the effects of CRD itself, leading to preserved youthful health and longer lifespan without the need to reduce food intake. A number of genes and pathways have been shown to be involved the actions of CRD in model organisms and these represent attractive targets for drug discovery and for developing CRM. CRD and its metabolic effects and the affected pathways as well as potential CRM are known to the skilled person, as is evident from the review article by Nikolai et al (Energy restriction and potential energy restriction mimetics. Nutrition Research Reviews (2015), 28, 100-120).

Furthermore, fasting mimicking diet product of the invention comprise kits and meal packages, which may be labeled as therapeutic meal packages, providing specific food and beverages to a subject. Such packages for providing foods and beverages, for example in a prepacked form, provide a fasting mimicking diet. In the context of the invention, it can be understood that such kits and meal packages are comprised by both the term CRD and the term CRM. The diet provided by such products may be a reduced diet or a diet that has similar effects as a reduced diet due to its specific composition. Fasting mimicking diet products are known and can be identified by the skilled person, for example as described in WO 2014/127000 A2 and WO 2009/132320 A2.

In embodiments, a CRM of the invention is or comprises a chemical compound or a drug. A CRM of the invention can be selected from the group comprising resveratrol, metformin, oxaloacetate, rimonabant, lipoic acid, 2-deoxy-D-glucose and rapamycin. In embodiments, a CRM of the invention can be selected from the group consisting of resveratrol, metformin, oxaloacetate, rimonabant, lipoic acid, 2-deoxy-D-glucose and rapamycin.

Downstream-type CRMs act on an intracellular signaling system and exert the same effect as CRD on downstream pathways, such as activation of the AMPK (AMP-activated protein kinase), inhibition of mTOR (mammalian target of rapamycin), activation of Sirtuins, and modifying the redox balance. Examples of downstream-type CRMs include, without limitation, Metformin (antidiabetic drug; AMPK activation), Rapamycin (immunosuppressant drug; mTOR inhibition), Resveratrol (food component; Sirtuin activation), Polyamines (food component; Epigenetic control) and Oxaloacetic acid (dietary supplement; Redox balance).

In contrast, an upstream-type CRM uses a mechanism of action targeting the energy metabolism system and transmitting a signal in the upstream direction to mimic CR, such as, without limitation, Chitosan (dietary supplement; Glucose diminution), Acarbose (antidiabetic drug; Glycosidase inhibition), 2-Deoxy-D-glucose (anticancer drug; Glycolysis inhibition), D-Glucosamine (dietary supplement; Glycolysis adjustment), D-Allulose (food component; Glycolysis improvement), SGLT2 inhibitor (antidiabetic drug; Glucose excretion). Upstream-type CRMs could be classified as compounds with energy metabolism inhibition effects, particularly glucose metabolism modulation effects. It can be difficult to strictly classify all CRMs as belonging to either type.

In embodiments, the CRM of the invention is or comprises metformin. Metformin is a drug for treating diabetes and is recommended as the first-line drug for type 2 diabetes in the guidelines of the American Diabetes Association and European Association for the Study of Diabetes. It currently is also being tested whether Metformin could also improve health in non-diabetic elderly people and the preliminary results indicate that metformin alone cannot trigger mitochondrial activation ion older organisms, which indicates that the therapy would not be effective in triggering health promoting stress response in the elderly. Instead, the combination of metformin with NAD-precursor supplementation has a very strong capacity to induce mitochondria activity in old individuals and thus has the potential to activate health benefits by inducing health-promoting stress responses. At least some of the effects of metformin are mediated by AMP-activated protein kinase (AMPK). Metformin transiently inhibits the mitochondrial respiratory chain (specifically, complex I), increases the intracellular AMP/ATP ratio, and activates AMPK. AMPK generally promotes catabolic reactions that produce ATP and suppresses anabolic reactions that consume ATP. Due to its ability to induce mitochondria stress, in particular through induction of AMPK signaling, metformin can be classified as a mitochondria stress-inducing CRM. In the liver, gluconeogenesis and fatty acid synthesis are suppressed, while β-oxidation is promoted. In the skeletal muscles and adipose tissue, AMPK promotes the translocation of glucose transporter type 4 to the cell membrane and stimulates sugar uptake. Although the mechanism is unknown, suppression of sugar absorption from the intestinal tract may contribute to the hypoglycemic effects of metformin. Administration of metformin to experimental animals results in different effects depending on the animal species. In the nematode Caenorhabditis elegans, metformin slowed the accumulation of lipofuscin, extended the median lifespan, and prolonged a youthful locomotory ability in a dose-dependent manner. It was also shown in C. elegans that metformin may prolong the lifespan through a mitochondrial process and that the production of metformin-induced reactive oxygen species (ROS) induced a signaling cascade that increased the overall lifespan. In C. elegans, both target of rapamycin complex 1 (TORC1) inhibition and AMPK activation contributed to the lifespan-extending effect of metformin. Genetic screening of C. elegans revealed that metformin was inactivated by inactivation of TORC1, a mechanism conserved from invertebrates to humans. In Drosophila, administration of metformin caused activation of AMPK and reduced the weight of adipose tissue. In C57BL/6 mice and in C. elegans administration of metformin resulted in an increased survival rate.

In embodiments, the CRM of the invention is or comprises rapamycin. Rapamycin (International Nonproprietary Name: sirolimus) is a macrolide compound produced by Streptomyces hygroscopicus and is the most widely used inhibitor of mammalian target of rapamycin (mTOR). Rapamycin exerts substantial regulatory effects on important biological processes such as proliferation and inflammation. Due to its ability to inhibit immune responses, rapamycin has been used clinically to prevent transplant rejection and treat autoimmune diseases. Rapamycin inhibits interleukin-2 signaling and other cytokine-receptor-dependent signaling pathways by acting on mTOR and prevents the activation of T and B cells. Its mechanism of action, which is similar to that of tacrolimus, involves binding to the cytoplasmic protein FK-binding protein 12 (FKBP12). However, unlike the tacrolimus-FKBP12 complex which inhibits calcineurin (also known as protein phosphatase 2B), the rapamycin-FKBP12 complex inhibits the mTOR pathway by binding directly to mTOR complex 1.

It was shown that that the mean lifespan of nematodes can be increased by treatment with rapamycin. The mTOR pathway, along with sirtuin family proteins and the insulin/insulin-like growth factor signaling pathway, is an important pathway that regulates lifespan, which can be targeted by CRMs of the present invention. In a Drosophila sod1 (superoxide dismutase 1 gene) mutant, rapamycin extends the average lifespan by 6% in males and 26% in females maintained on standard feed. Similarly, rapamycin promotes survival in a Drosophila model of a mitochondrial disease. Furthermore, rapamycin prolongs the lifespan of mature mice by 28-38% compared to control animals. Mature mice are about 20-months old, corresponding to approximately 60 years of age in humans, suggesting that rapamycin can extend life expectancy in already aged humans. It was also shown that three months of rapamycin treatment in middle-aged mice increased the average lifespan by 60%, improving the health status of middle-aged and elderly mice. Low-dose rapamycin also extends the lifespan of mouse models with mitochondrial disorders. Additionally, it was shown that rapamycin treatment has a beneficial effect on the arterial function of old mice, and these improvements are associated with decreased expression of proteins involved in oxidative stress, and cell cycle control. It was also shown that rapamycin has beneficial effects on neoplastic diseases, delayed multiple aspects of mouse aging, and extended longevity.

In embodiments, the CRM of the invention is or comprises resveratrol. Resveratrol is a natural polyphenolic phytoalexin mainly present in the skin of grapes and in red wine. This polyphenol has been most thoroughly studied as a compound that activates sirtuin 1 or its invertebrate homologs and is known to protect living organisms against ROS and was shown to exert its antioxidant effects by activating SIRT2 to deacetylate peroxiredoxin 1. Resveratrol has been reported to prolong the lifespan of several different species. For example, treating C. elegans with resveratrol prolongs its lifespan via a mechanism SIR-2.1 dependent mechanism. For example, resveratrol prolongs the lifespan of C. elegans in vivo under oxidative stress. Also, it was shown that at a concentration of 400 μM, resveratrol extended the lifespan of female Drosophila fed a high-fat diet by 10%-15%. Also, extension effects on the mean lifespan were observed when resveratrol was administered to obese mice induced by a high-fat diet. Resveratrol also preserved indices of vascular function in normal rats, although without extending the lifespan. Furthermore, in clinical studies, intake of resveratrol improved the memory capacity of elderly subjects and improved the blood lipid levels and glucose control in obese and adult diabetic subjects. Furthermore, it was observed that resveratrol improved vascular function, particularly in elderly people. Pterostilbene, a naturally occurring analog of resveratrol, has been suggested to function as a Sirtuin activator.

In embodiments, the CRM of the invention is or comprises polyamines. It is known that polyamine production by gut bacteria promotes longevity in mice. A preferred polyamine is spermidine, which has been shown to prolong life of the lifespan of yeasts, worms, flies and mice, even in middle-aged mice. Polyamines such as spermidine enhance autophagy leading to reduction of oxidative stress in these models and mice. Polyamines and in particular spermidine are cardioprotective and neuroprotective and stimulate anticancer immune surveillance. In humans, consumption of polyamines in food is correlated with reduced cardiovascular- and cancer-related mortality.

A further preferred CRM of the invention is oxaloacetic acid. Oxaloacetic acid is an intermediate of the Kreb's cycle and is related to Nicotinamide adenine dinucleotide (NAD+) levels and redox balance in cells. In C. elegans, oxaloacetic acid prolongs the lifespan independently of sirtuin but this effect depends on AMPK. In humans, a decrease of blood glucose levels can be observed following administration of oxaloacetic acid. Due to its ability to induce mitochondria stress, in particular through induction of AMPK signaling, oxaloacetic acid can be classified as a mitochondria stress-inducing CRM.

In embodiments, the CRM of the invention is or comprises 2-Deoxy-D-Glucose (2DG). 2DG is a glucose derivative in which the 2-hydroxyl group is replaced by a hydrogen atom. 2DG is not metabolized via glycolysis and was the first proposed dietary restriction mimetic. It is thought to delay age-related dysfunctions and extend the lifespan by suppressing glycolytic activity. 2DG has been suggested to prolong lifespan through “mitochondrial hormesis” or “mitohormesis”, which is a concept proposing that induction of mitochondrial metabolism may induce a positive response to increased formation of ROS and other related stressors, leading to a secondary (i.e., hermetic) increase in stress defense, resulting in reduced net stress levels. Reduction of glycolysis by 2DG induces the utilization of stored fat and mitochondrial respiration via AMPK. Due to its ability to induce mitochondria stress, in particular through induction of AMPK signaling, 2DG can be classified as a mitochondria stress-inducing CRM. 2DG has been used in many studies focused on the impact of reduced metabolic rates and showed great potential as a CRM. For example, in a study comparing the effects of 2DG and CR in rodents, 2DG administration showed the same effects on locomotory activity, heart rate, and blood pressure as CR administration. In summary, 2DG shows very similar effects as CR.

Rimonabant (Acomplia) is another CRM of the present invention. It is an anti-obesity drug approved for use in the European Union. This is an endocannabinoid-1 receptor blocker. Endocannabinoids are cannabis-like chemicals that stimulate appetite and also regulate energy balance. Overstimulation of the endocannabinoid receptor in the hypothalamus promotes appetite and stimulates lipogenesis. It also blocks the beneficial actions of adiponectin. Rimonabant inhibits these and so it reduces appetite, balances energy, and increases adiponectin, which reduces intra-abdominal fat. It improves lipid profile, glucose tolerance, and waist measurement. Therefore, it has similar effects as CR. Consequently, rimonabant acts similarly to CRD and is an inducer of mitochondrial stress, and can therefore be categorized as a mitochondria stress-inducing CRM in the sense of the present invention.

In preferred embodiments, the present invention relates to the combined administration of a NAD-precursor together with CRM that act as mitochondria stress-inducing CRMs, such as metformin, metformin, 2-deoxy-D-glucose, oxaloacetate, rimonabant. Particularly preferred are mitochondria stress-inducing CRMs that mainly act through the activation of AMPK signaling, including in particular metformin, 2-deoxy-D-glucose, and oxaloacetate. Such mitochondria stress-inducing CRMs can be differentiated from CRMs that are acting mainly through activation of Sirtuin signaling and/or through induction of autophagy, such as pterostilbene, resveratrol and spermidine. In embodiments, the CRMs of the invention are CRMs that induce mitochondria stress and preferably act through AMPK signaling on mitochondria function, whereas preferably Sirtuin signaling and/or induction of autophagy are not affected or not strongly affected.

Lipoic Acid (α-Lipoic Acid, Alpha Lipoic Acid, or ALA) is another CRM in the sense of the present invention. Further CRM of the present invention comprise, without limitation, acarbose, D-glucosamine, D-allulose, sodium-glucose cotransporter 2 inhibitors, chitosan, peroxisome proliferator-activated receptor gamma inhibitors, agents that modulate sirtuins (called STAC-sirtuin-activating compounds), for example fisetin, Exanadin (exenatide), Adiponectin, Acipimox, Hydroxycitrate, Dipeptidyl peptidase 4 (DPP-4) inhibitors, lodoacetate, Mannoheptulose (glycolytic inhibitor), modulators of neuropeptide Y (NPY), 4-Phenylbutyrate (PBA), and Gymnemoside (modulates glucose absorption).

Metabolic Plasticity and Mitochondrial Metabolism

In embodiments of the invention, CRD and/or CRM promote metabolic plasticity and/or metabolic stress responses by induction of mitochondrial metabolism. In further embodiments, NAD precursor enhances and/or restores the ability of cells of a middle-aged or old individual to respond to CRD and/or CRM. In embodiments, the NAD precursor restores mitochondrial functions of cells of old or middle-aged individuals, which have already been impaired or declined due to aging in comparison to its previous function. In embodiments, the NAD precursor enhances and/or restores a therapeutic response of a human subject, more preferably a subject over 40 years old, to CRD and/or CRM administration, by increasing mitochondrial function and the responsiveness of mitochondria to increase activity in response to CRD and/or CMD.

As used herein, the term metabolic plasticity refers to the ability of subjects, organisms or cells to switch or adapting its metabolism in response to changes in nutrient availability, such as in response to CRD and/or CRM.

In general, metabolism is the set of life-sustaining chemical reactions in organisms. The three main purposes of metabolism are the conversion of food to energy to run cellular processes; the conversion of food/fuel to building blocks for proteins, lipids, nucleic acids, and some carbohydrates; and the elimination of nitrogenous wastes. These enzyme-catalyzed reactions allow organisms to grow and reproduce, maintain their structures, and respond to their environments. Metabolic reactions may be categorized as catabolic—the breaking down of compounds (for example, the breaking down of glucose to pyruvate by cellular respiration); or anabolic—the building up (synthesis) of compounds (such as proteins, carbohydrates, lipids, and nucleic acids). Usually, catabolism releases energy, and anabolism consumes energy. The chemical reactions of metabolism are organized into metabolic pathways, in which one chemical is transformed through a series of steps into another chemical, each step being facilitated by a specific enzyme. Enzymes are crucial for metabolism because they allow organisms to drive desirable reactions that require energy that will not occur by themselves, by coupling them to spontaneous reactions that release energy. Enzymes act as catalysts—they allow a reaction to proceed more rapidly—and they also allow the regulation of the rate of a metabolic reaction, for example in response to changes in the cell's environment or to signals from other cells.

Cellular respiration is a set of metabolic reactions and processes that take place in the cells of organisms to convert biochemical energy from nutrients into adenosine triphosphate (ATP), and then release waste products. The reactions involved in respiration are catabolic reactions, which break large molecules into smaller ones, releasing energy in the process, as weak so-called “high-energy” bonds are replaced by stronger bonds in the products. Respiration is one of the key ways a cell releases chemical energy to fuel cellular activity. Cellular respiration is considered an exothermic redox reaction which releases heat. The overall reaction occurs in a series of biochemical steps, most of which are redox reactions themselves. Although cellular respiration is technically a combustion reaction, it clearly does not resemble one when it occurs in a living cell because of the slow release of energy from the series of reactions. Nutrients that are commonly used by animal and plant cells in respiration include sugar, amino acids and fatty acids, and the most common oxidizing agent (electron acceptor) is molecular oxygen (O2). The chemical energy stored in ATP (its third phosphate group is weakly bonded to the rest of the molecule and is cheaply broken allowing stronger bonds to form, thereby transferring energy for use by the cell) can then be used to drive processes requiring energy, including biosynthesis, locomotion or transportation of molecules across cell membranes.

Aerobic respiration requires oxygen (O2) in order to create ATP. Although carbohydrates, fats, and proteins are consumed as reactants, it is the preferred method of pyruvate breakdown in glycolysis and requires that pyruvate enter the mitochondria in order to be fully oxidized by the Krebs cycle. The products of this process are carbon dioxide and water, but the energy transferred is used to break bonds in ADP as the third phosphate group is added to form ATP (adenosine triphosphate), by substrate-level phosphorylation, NADH and FADH2. The potential of NADH and FADH2 is converted to more ATP through an electron transport chain with oxygen as the “terminal electron acceptor”. Most of the ATP produced by aerobic cellular respiration is made by oxidative phosphorylation. This works by the energy released in the consumption of pyruvate being used to create a chemiosmotic potential by pumping protons across a membrane. This potential is then used to drive ATP synthase and produce ATP from ADP and a phosphate group.

In the context of the present invention, metabolic plasticity in particular relates to the process of energy transformation and the ability to switch or adapting between different mechanism of energy transformation, in particular, the switching between oxidative phosphorylation and glycolysis. Metabolic plasticity can be assessed by measuring responses of cells or organisms to for example changes in nutrient availability or exposure to other stimuli. For example, the body weight of a subject can be monitored in response to changed nutrient availability. Furthermore, the oxygen consumption and/or the extracellular acidification rate of cells can be assessed in order to determine the contribution of mitochondrial respiration/oxidative phosphorylation and glycolysis to the metabolism of a cell. This can be done by using the so-called Seahorse-technology of Agilent or other techniques know to the skilled person for measuring oxidative phosphorylation and glycolysis in cells and the rate or ratio of the respective pathways. Furthermore, metabolic plasticity can be determined by measuring certain molecules participating in specific metabolic processes, such as lactate or pyruvate, or activity or expression of enzymes participating in specific metabolic pathways or processes, such as lactate dehydrogenase (LDH) or pyruvate dehydrogenase A and B (PDHA1 and PDHB).

In embodiments, the term metabolic plasticity refers to the ability of an individual to metabolically adapt to a to a change in diet, in particular to a change of its diet to CRD, in order to increase metabolic efficiency of cells, to stabilize body weight, and to improve the function of stem cells and differentiated cells in response to change availability of nutrients, in particular reduction in nutrient availability. This is a capacity that old individuals tend to lose, which results in a loss of body weight in response to reduction of nutrient availability. Accordingly, metabolic plasticity can be measured by monitoring body weight, mitochondrial respiration, and stem cell function after provision of a CRD.

As used herein, the term “mitochondria metabolism” relates to the process of mitochondria respiration (oxidative phosphorylation). Mitochondria have a central role in energy metabolism. Part of the free energy derived from the oxidation of food is inside mitochondria transformed to ATP, which depends on oxygen. When oxygen is limited, glycolytic products are metabolized directly in the cytosol by the less efficient anaerobic respiration that is independent of mitochondria. The mitochondrial ATP production relies on the electron transport chain (ETC), composed of respiratory chain complexes I-IV, which transfer electrons in a stepwise fashion until they finally reduce oxygen to form water. The NADH and FADH2 formed in glycolysis, fatty-acid oxidation and the citric acid cycle are energy-rich molecules that donate electrons to the ETC. Electrons move toward compounds with more positive oxidative potentials and the incremental release of energy during the electron transfer is used to pump protons (H+) into the intramembrane space. Complexes I, III and IV function as H+ pumps that are driven by the free energy of coupled oxidation reactions. During the electron transfer, protons are always pumped from the mitochondrial matrix to the intermembrane space, resulting in a potential of ˜150-180 mV. Proton gradient generates a chemiosmotic potential, also known as the proton motive force, which drives the ADP phosphorylation via the ATP synthase (FoF1 ATPase—complex V). Fo domain of ATPase couples a proton translocation across the inner mitochondrial membrane with the phosphorylation of ADP to ATP. The rate of mitochondrial respiration depends on the phosphorylation potential expressed as a [ATP]/[ADP] [Pi] ratio across the inner mitochondrial membrane that is regulated by the adenine nucleotide translocase (ANT). In the case of increased cellular energy demand when the phosphorylation potential is decreased and more ADP is available, a respiration rate is increased leading to an increased ATP synthesis. There is usually a tight coupling between the electron transport and the ATP synthesis and an inhibition of ATP synthase will therefore also inhibit the electron transport and cellular respiration. Under certain conditions, protons can re-enter into mitochondrial matrix without contributing to the ATP synthesis and the energy of proton electrochemical gradient will be released as heat. This process, known as proton leak or mitochondrial uncoupling, could be mediated by protonophores (such as FCCP) and uncoupling proteins (UCPs). As a consequence, uncoupling leads to a low ATP production concomitant with high levels of electron transfer and high cellular respiration.

As used herein, an increase in mitochondrial metabolism and an increased mitochondrial function in particular refer to an increased rate of mitochondrial respiration/oxidative phosphorylation. Mitochondrial metabolism is an indicator of mitochondrial function and can be analyzed for example by measuring the rate of oxidative phosphorylation, the mitochondrial membrane potential (MtMP), cellular levels of reactive oxygen species (ROS), wherein an increased rate of oxidative phosphorylation, a high mitochondrial membrane potential (MtMP), and low levels of reactive oxygen species (ROS) are indicative of functional mitochondria and a high or intact mitochondrial metabolism. Also, NADH and NADPH levels can be determined as an indicator of mitochondrial function and metabolism, wherein high levels are indicative of good functionality.

Further indicators of mitochondrial functionality and metabolism are expression levels of genes that are centrally involved in mitochondrial function and biogenesis, which include nuclear and mitochondrial genes, such as Nrf1, Tfam, Nd1, Cytb, Co1 and Atp6, among others known to the skilled person. In contrast, a (concomitant) upregulation of glycolytic enzymes can be indicative of a declining mitochondrial metabolism. Furthermore, high ATP levels are an indicator of intact mitochondrial function and mitochondrial metabolism. A declined of mitochondrial function can be observed by determining the parameters above and comparing them to a previously determined value or other reference values.

Mitochondrial metabolism is the key function of mitochondria to generate ATP, which is needed for a variety of biological processes in cells. This process involves oxidation of Acetly-Coenzyme-A in tricarboxylic acid (TCA) cycle. The process converts NAD into NADH, which is then used during oxidative phosphorylation in the electron transport chain (ETC) pathway to synthesize ATP—a molecule that stores energy chemically. Mitochondrial metabolism is the main function of mitochondria, which can be activated or reduced. This depends on the energy demand of cells/organism as well as on food availability. If mitochondrial function increases, it means that mitochondrial metabolism becomes more active and more efficient. This leads to an increase in ATP production and to a change in the ration of NAD to NADH, which will lead to induction of metabolic stress response pathways, such as the activation of sirtuins.

In the context of the present invention, the term “metabolic stress response” relates to the reaction of a cell or organism in response to metabolic stress, such as nutrient deprivation.

Nutrient deprivation is sensed by nutrient sensing pathways and leads to activation or modulation of certain signaling pathways, such as the mammalian target of rapamycin (mTOR) and integrated stress response (ISR) pathways. The mTOR signaling pathway controls major growth promoting signals and the pathway is suppressed in response to decreases in nutrient availability. Furthermore, gene regulation of metabolic stress response genes, such as Nrf1 and Hif1α, can be observed. Further metabolic stress response genes are known in the art and can be identified by a skilled person, for example the set of 52 metabolic stress response genes disclosed in the examples provided herein. Biological pathways involved in a metabolic stress response and corresponding genes include, without limitation, autophagy and mitophagy induction (Parkin, Sirt1), cell cycle control (p57), mitochondrial activity and biogenesis (Polg, Ucp2, ldh1, Cox4i1), chromatin remodeling and mtUPR (Sirt7), insulin/Igf signaling (Hmga2, Irs2), and NAD biosynthesis (Naprt, which catalyzes the first step in the biosynthesis of NAD from nicotinic acid), apoptosis, and unfolded protein responses.

In general, metabolic stress is induced when there are changes in nutrient availability, either a lack of nutrients or an overload of the organism with nutrients. In both scenarios the cells and the organism have to adapt in order to either ensures a more efficient usage of nutrients (in case nutrients become limiting) or to store nutrients (in the scenario of a surplus in nutrient availability to ensure saving of nutrient for times of reduced nutrient availability). It has been recognized that the metabolic stress responses that occur in response to a lack of nutrient availability (such as CDR or CMD) increase cellular and organism fitness thereby counteracting aging-associated decreases in cell and organism function.

Therapeutic Application of the Invention

As used herein, the term “subject” means a human or non-human animal selected for treatment or therapy. The phrases “therapeutically-effective amount” and “effective amount” of a NAD precursor or CRM of the invention as used herein means the amount of an agent which is effective for producing the desired therapeutic effect in at least a sub-population of cells in a subject at a reasonable benefit/risk ratio applicable to any medical treatment.

Preferably, in the context of the present the subject is a mammal, more preferably human. In further preferred embodiments, the subject is an old and/or a middle-aged subject, which may be a human subject that is 40 years old or older.

In embodiments of the invention, the subject is old. In embodiments of the invention, the subject is old or middle-aged. As used herein, the term “old” in the context of, for example, a person, patient, individual or living organism, refers to an individual which has reached or passed the age representing half of the expected, average or median life expectancy of the respective species. For example, if one considers 80 years the average life expectancy of humans, an old individual would be a person who has reached an age of 40 years or more. In preferred embodiments, an old human is 40 year of age or older. In the context of the invention, a middle-aged individual is in the mid third of its expected lifespan. For example, if one considers 80 years the average lifespan of humans, an person with an age in the range of about 26.5 to 53 years is considered a middle-aged person.

In embodiments, an old human subject or individual is 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 years old or older. In preferred embodiments of the invention, an old human subject is 40 years old or older. In further embodiments, an old human subject is 45 years or older. In further embodiments, an old human subject is 50 years or older. In further embodiments, an old human subject is 55 years or older. In further embodiments, an old human subject is 60 years or older. In further embodiments, an old human subject is 60 years or older. In further embodiments, an old human subject is 65 years or older. In further embodiments, an old human subject is 70 years or older.

In embodiments, a middle-aged human subject is 25 years or older. In embodiments, a middle-aged human subject is 30 years or older. In embodiments, a middle-aged human subject is 35 years or older. In embodiments, a middle-aged human subject is 40 years or older. In embodiments, a middle-aged human subject is 45 years or older. In embodiments, a middle-aged human subject is 50 years or older. In embodiments, a middle-aged human subject is 55 years or older. In further embodiments, a middle-aged human subject is 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 years old or older.

The present invention is directed to the treatment and/or prevention of an age-related medical condition in a subject.

As used herein, “treatment” or “therapy” generally means to obtain a desired pharmacological effect and/or physiological effect. The effect may be prophylactic in view of completely or partially preventing a disease and/or a symptom, for example by reducing the risk of a subject having a particular disease or symptom, or may be therapeutic in view of partially or completely curing a disease and/or adverse effect of the disease. In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

Age-Related Medical Conditions

In particular, the treatment described herein relates to either treating or preventing age-related medical conditions, comprising slowing, reversing and/or inhibiting the ageing process, wherein in preferred embodiments mitochondrial function is enhance or reactivated or a decline of mitochondrial function is prevented, preferably by use of an NAD precursor, such as NR, which in turn reinstalls metabolic plasticity, specifically the responsiveness of mitochondrial metabolism and/or metabolic stress responses to be activated by CRD and/or CRM. CDR and CRM are prophylactic treatments in order to prevent or slow down age-related processes is intended to encompass prevention or reduction of risk of developing an age-related condition. However, it was recently recognized that these interventions fail during aging (Hahn et al. 2019). The here described invention solves this problem by identifying that the combination of the NAD-precursor (NR) along with co-application of CRD and/or CRM reactivates metabolic plasticity and the responsiveness of cells and tissues to enhance mitochondrial metabolism and cellular functionality.

As used in the context of the present invention, the term age-related medical condition comprises aging-associated diseases, aging-associated dysfunctions, such as aging-associated organ dysfunctions, and conditions associated with a decline in mitochondrial function.

Age-related medical conditions are changes in the health status of a subject that occur with age due to changes in organ and cell functions that depend on the age of the subject. During aging the incidence of acute and chronic conditions such as neurological disorders, diabetes, degenerative arthritis, and cancer rises within individuals, so that aging has been termed the substrate on which age-associated diseases grow. The invention therefore relates to prophylactic and symptomatic treatment of diseases associated with ageing.

The molecular pathways underlying aging are only partially understood, as large individual heterogeneity of the biological aging process is observed. These inter-individual differences are proposed to derive from accumulation of stochastic damage that is counteracted by genetically encoded and environmentally regulated repair systems. Aging associated mitochondrial dysfunction by itself is thought to contribute to stem cell and tissue aging. The here described inventions identifies a new role for aging associated mitochondrial dysfunction in limiting the potential of CRD and/or CRM to enhance organism fitness and to delay aging by increasing mitochondrial activity and the induction of metabolic stress responses. The invention shows that application of an NAD-precursor in combination CRD and/or CRM has the potential to reinstall the induction of mitochondrial activity and metabolic stress responses in response to CRD and/or CRM. The present invention therefore provides means for the treatment and/or prevention and/or reduction in risk of ageing as such, in addition to age-related medical conditions.

As used herein, an aging associated disease is a disease that is most often seen with increasing frequency with increasing age of the subject or patient. Essentially, aging-associated diseases are complications arising from aging or senescence. “Aging-associated disease” is used here to mean “diseases of the elderly”, so diseases incurring with higher frequency in older individuals. Non-exhaustive examples of aging-associated diseases are atherosclerosis and cardiovascular disease, cancer, arthritis, cataracts, osteoporosis, type 2 diabetes, hypertension and neurodegenerative diseases, such as Alzheimer's disease. The incidence of such aging associated diseases increases exponentially with age.

The main risk factor of cardiovascular diseases (CVD) is age, which is why cardiovascular disease are considered aging associated disease of the present invention. CVD is a class of diseases that involve the heart or blood vessels. CVD includes coronary artery diseases such as angina and myocardial infarction (heart attack). Other CVDs include, without limitation, stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis.

Aging associated diseases of the heart include coronary artery disease, CHD, cardiomyopathy, valvular heart disease, pericardial disease, congenital heart disease (e.g., coarctation, atrial or ventricular septal defects), and heart failure. Diseases of the blood vessels may include arteriosclerosis, atherosclerosis, hypertension, stroke, vascular dementia, aneurysm, peripheral arterial disease, intermittent claudication, vasculitis, venous incompetence, venous thrombosis, varicose veins, and lymphedema.

The term arteriosclerosis relates to an aging associated disease characterized by the pathological thickening, hardening and loss of elasticity of artery walls that can lead to stenosis and subsequent insufficient blood supply of downstream tissues resulting in ischemia. This process is often associated with calcification of the arterial wall. There are different types of arteriosclerosis that affect different anatomical locations and have different etiologies. Atherosclerosis is a specific type of arteriosclerosis, which is defined by the accumulation of white blood cells in the artery wall and formation of atheromatous plaques. Atherosclerosis is a chronic disease that can remain asymptomatic for extended periods until lumen stenosis of the affected artery occurs.

Additionally, ruptures of atherosclerotic lesions can lead to thrombus formation and subsequent thromboembolism, which can lead to tissue necrosis/infarction in all parts of the body. Dramatic examples of such events are myocardial infarction and stroke, these after-effects of atherosclerosis represent the most common cause of death in industrialized countries and therefore improved treatment strategies are urgently needed. Arteriosclerosis according to the present invention refers to, but is not limited to, one or more of, atherosclerosis, arteriosclerosis obliterans, and Monckeberg's arteriosclerosis.

Aging associated diseases of the invention comprise in particular circulatory disorders, cardiovascular disease, artery or blood vessel conditions and/or ischemic obstructive or occlusive diseases or conditions refer to states of vascular tissue where blood flow is, or can become, impaired or altered from normal levels. Many pathological conditions can lead to vascular diseases that are associated with alterations in the normal vascular condition of the affected tissues and/or systems. Examples of vascular conditions or vascular diseases to which the methods of the invention apply are those in which the vasculature of the affected tissue or system is senescent or otherwise altered in some way such that blood flow to the tissue or system is reduced or in danger of being reduced or increased above normal levels. It refers to any disorder in any of the various parts of the cardiovascular system, which consists of the heart and all of the blood vessels found throughout the body.

Neurodegenerative disease or neurodegeneration is a term for aging associated medical conditions in which the progressive loss of structure or function of neurons, including death of neurons, occurs. Many neurodegenerative diseases, including ALS, Parkinson's, Alzheimer's, and Huntington's, occur as a result of neurodegenerative processes. Such diseases are commonly considered to be incurable, resulting in progressive degeneration and/or death of neuron cells. A number of similarities are present in the features of these diseases, linking these diseases on a sub-cellular level. Some of the parallels between different neurodegenerative disorders include atypical protein assembly as well as induced cell death. Dementia is a group of brain diseases causing a gradual decline of cognitive functions. Most of these diseases are chronic neurodegenerative diseases and are associated with neurobehavioral and/or neuropsychiatric symptoms that disable patients to independently perform activities of daily live. Alzheimer's disease is the most common form of dementia with 25 million affected individuals worldwide in the year 2000. This number is expected to increase to 1 14 million cases in 2050, unless preventive or neuroprotective therapy approaches emerge. Dementia according to the present invention refers to, but is not limited to, one or more of, Alzheimer's disease, vascular dementia, post-stroke dementia, Lewy body dementia, frontotemporal dementia, Huntington's disease, and Creutzfeldt-Jakob disease.

Aging associated diseases comprise diabetes mellitus, which is a group of chronic metabolic diseases that are associated with high blood sugar levels over prolonged periods, which can lead to severe complications including cardiovascular diseases, stroke, kidney failure, foot ulcers and damaged eyes. The two main subtypes are type 1 and type 2 diabetes mellitus. Type 1 diabetes mellitus is characterized by the loss of insulin-producing cells in the pancreas. It accounts for about 10% of the diabetes cases in the US and Europe, mostly affects children and is often associated with autoimmune pathologies. Type 2 diabetes mellitus is characterized by insulin resistance. Diabetes mellitus represents a massive health issue with more than 350 million affected people in 2013 worldwide. Diabetes mellitus according to the present invention refers to, but is not limited to, one or more of, type 1 diabetes mellitus, type 2 diabetes mellitus, gestational diabetes, and latent autoimmune diabetes of adults. Autoimmune diseases are a group of diseases that are caused by an abnormal immune response of the body against specific molecules or cells that are normally present in the body and should therefore be tolerated by the immune system under physiological conditions. The pathological reaction of the body's immune system against its own components can lead to severe physical conditions. A large number of diseases have been identified as being caused by autoimmune reactions and many pathologies of unclear etiology are suspected to have autoimmune components and are therefore termed autoimmune-related diseases. Therefore, the development of effective and specific treatment strategies for this group of diseases is urgently needed.

Metabolic syndrome is another example of an aging associated disease of the invention. Metabolic syndrome is a clustering of at least three of the five following medical conditions: central obesity, high blood pressure, high blood sugar, high serum triglycerides, and low serum high-density lipoprotein (HDL). Metabolic syndrome is associated with the risk of developing cardiovascular disease and type 2 diabetes. The syndrome is thought to be caused by an underlying disorder of energy utilization and storage, including dysfunction of mitochondrial metabolism. The continuous provision of energy via dietary carbohydrate, lipid, and protein fuels, unmatched by physical activity/energy demand creates a backlog of the products of mitochondrial oxidation, a process associated with progressive mitochondrial dysfunction and insulin resistance.

Further aging associated disease of the invention comprise disease of the liver and the kidney, such as liver failure, nonalcoholic fatty liver disease (NAFLD), nonalcoholic steatohepatitis (NASH), chronic kidney disease, acute kidney injury, kidney failure.

Aging associated diseases also comprise neuropathy, often also referred to as peripheral neuropathy. Neuropathy is a disease affecting the peripheral nerves, meaning nerves beyond the brain and spinal cord. Damage to peripheral nerves may impair sensation, movement, gland or organ function depending on which nerves are affected; in other words, neuropathy affecting motor, sensory, or autonomic nerves result in different symptoms. More than one type of nerve may be affected simultaneously. Peripheral neuropathy may be acute (with sudden onset, rapid progress) or chronic (symptoms begin subtly and progress slowly), and may be reversible or permanent.

Muscle atrophy is another aging associated disease of the invention. It is characterized by the loss of skeletal muscle mass that can be caused by immobility, aging, malnutrition, medications, or a wide range of injuries or diseases that impact the musculoskeletal or nervous system. Sarcopenia is the muscle atrophy associated with aging and can be slowed by exercise. Finally, diseases of the muscles such as muscular dystrophy or myopathies can cause atrophy, as well as damage to the nervous system such as in spinal cord injury or stroke. Muscle atrophy results from an imbalance between protein synthesis and protein degradation, although the mechanisms are incompletely understood and are variable depending on the cause. Muscle loss can be quantified with advanced imaging studies, but this is not frequently pursued.

Sarcopenia is an aging associated disease of the invention characterized by the degenerative loss of skeletal muscle mass, quality, and strength associated with aging and immobility. The rate of muscle loss is dependent on exercise level, co-morbidities, nutrition and other factors. Sarcopenia can lead to reduction in functional status and cause disability. The muscle loss is related to changes in muscle synthesis signaling pathways. It is distinct from cachexia, in which muscle is degraded through cytokine-mediated degradation, although both conditions may co-exist. Sarcopenia is considered a component of the frailty syndrome. Changes in hormones, immobility, age-related muscle changes, nutrition and neurodegenerative changes have all been recognized as potential causative factors.

The term “cancer” comprises a group of diseases that can affect any part of the body and is caused by abnormal cell growth and proliferation. These proliferating cells have the potential to invade the surrounding tissue and/or to spread to other parts of the body where they form metastasis. The incidence of cancer in increasing with age and cancer is therefore considered an aging associated disease of the present invention. Cancer according to the present invention refers to all types of cancer or neoplasm or malignant tumors found in mammals, including leukemias, sarcomas, melanomas and carcinomas. Examples of cancers are cancer of the breast, pancreas, colon, lung, non-small cell lung, ovary, and prostate.

Leukemias include, but are not limited to acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Sarcomas include, but are not limited to a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Melanomas include, but are not limited to include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

Carcinomas include, but are not limited to acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma exulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticurn, carcinoma telangiectodes, transitional cell carcinoma, carcinoma tuberosum, tuberous carcinoma, verrucous carcinoma, and carcinoma villosum.

Additional cancers include, but are not limited to Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, cervical cancer, endometrial cancer, adrenal cortical cancer, and prostate cancer.

In embodiments, the age-related condition is an aging associated dysfunction of cellular functions, such as a dysfunction of mitochondrial metabolism or other cellular mechanisms that lead to cellular and ultimately organ dysfunction leading to a clinical manifestation, such as an aging associated disease.

Many aging associated diseases are also associated with a decline in mitochondrial function. This group comprises in particular myocardial dysfunction, myocardial infarction, heart failure, liver failure, nonalcoholic fatty liver disease (NAFLD), nonalcoholic steatohepatitis (NASH), chronic kidney disease, acute kidney injury, kidney failure, muscle atrophy, sarcopenia, cardiomyopathy, cardiovascular disease, cancer, diabetes, metabolic syndrome, neuropathies, neurodegenerative disorders such as amyotrophic lateral sclerosis (ALS), multiple sclerosis, Parkinson's disease, and Alzheimer's disease.

The Pharmaceutical Combination of the Invention

According to the present invention, a “pharmaceutical combination” is the combined presence of an NAD precursor with a CRM, i.e. in proximity to one another. In one embodiment, the combination is suitable for combined administration. In one embodiment, the pharmaceutical combination as described herein is characterized in that the NAD precursor is in a pharmaceutical composition in admixture with a pharmaceutically acceptable carrier, and the CRM is in a separate pharmaceutical composition in admixture with a pharmaceutically acceptable carrier. The pharmaceutical combination of the present invention can therefore in some embodiments relate to the presence of two separate compositions or dosage forms in proximity to each other.

The agents in combination are not required to be present in a single composition. In one embodiment, the pharmaceutical combination as described herein is characterized in that the NAD precursor and the CRM according to any one the embodiments of the invention are present in a kit, in spatial proximity but in separate containers and/or compositions. The production of a kit lies within the abilities of a skilled person. In one embodiment, separate compositions comprising two separate agents may be packaged and marketed together as a combination. In other embodiments, the offering of the two agents in combination, such as in a single catalogue, but in separate packaging is understood as a combination.

In one embodiment, the pharmaceutical combination as described herein is characterized in that the NAD precursor and the CRM according to the embodiments of the invention are combined in a single pharmaceutical composition in admixture with a pharmaceutically acceptable carrier. Combination preparations or compositions are known to a skilled person, who is capable of assessing compatible carrier materials and formulation forms suitable for both agents in the combination.

It is a surprising advantage of the pharmaceutical composition that the NAD precursor and the CRM act synergistically to enable a strong benefit for the recipient, especially for an old subject. To determine or quantify the degree of synergy or antagonism obtained by any given combination, a number of models may be employed. Typically, synergy is considered an effect of a magnitude beyond the sum of two known effects. In some embodiments, the combination response is compared against the expected combination response, under the assumption of non-interaction calculated using a reference model (refer Tang J. et al. (2015) What is synergy? The saariselks agreement revisited. Front. Pharmacol., 6, 181).

In embodiments, the NAD precursor and the CRM and/or CRD are provided to subject by combined administration. According to the present invention, the term “combined administration”, otherwise known as co-administration or joint treatment, encompasses in some embodiments the administration of separate formulations of the compounds described herein, whereby treatment may occur within 30 minutes of each other, in the same hour, on the same day, in the same week or in the same month as one another. Alternating administration of two agents is considered as one embodiment of combined administration. Staggered administration is encompassed by the term combined administration, whereby one agent may be administered, followed by the later administration of a second agent, optionally followed by administration of the first agent, again, and so forth. Simultaneous administration of multiple agents is considered as one embodiment of combined administration. Simultaneous administration encompasses in some embodiments, for example the taking of multiple compositions comprising the multiple agents at the same time, e.g. orally by ingesting separate tablets simultaneously. A combination medicament, such as a single formulation comprising multiple agents disclosed herein may also be used in order to co-administer the various components in a single administration or dosage.

A combined therapy or combined administration of one agent may precede or follow treatment with the other agent to be combined, by intervals ranging from minutes to weeks. In embodiments where the second agent and the first agent are administered separately, one would generally ensure that a significant period of time did not expire between the time of each delivery, such that the first and second agents would still be able to exert an advantageously combined synergistic effect on a treatment site. In such instances, it is contemplated that one would contact the subject with both modalities within about 12-24 h of each other and, more preferably, within about 6-12 h of each other, with a delay time of only about 12 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In the meaning of the invention, any form of administration of the multiple agents described herein is encompassed by combined administration, such that a beneficial additional therapeutic effect, preferably a synergistic effect, is achieved through the combined administration of the two agents. The present invention also relates to a pharmaceutical composition comprising the compounds described herein.

The invention also relates to pharmaceutically acceptable salts of the compounds described herein, in addition to enantiomers and/or tautomers of the compounds described. The term “pharmaceutical composition” refers to a combination of the agent as described herein with a pharmaceutically acceptable carrier. The phrase “pharmaceutically-acceptable” refers to molecular entities and compositions that do not produce a severe allergic or similar untoward reaction when administered to a human. As used herein, “carrier” or “carrier substance” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions. Tablets contain the active ingredient in admixture with non-toxic pharmaceutically acceptable excipients which are suitable for the manufacture of tablets. The tablets may be uncoated or they may be coated by known techniques to delay disintegration and absorption in the gastrointestinal tract and thereby provide a sustained action over a longer period.

Dosage levels of the order of from about 0.01 mg to about 500 mg per kilogram of body weight per day are useful in the treatment of the indicated conditions. For example, an age-related medical condition may be effectively treated by the administration of from about 0.01 to 50 mg of the inventive molecule per kilogram of body weight per day (about 0.5 mg to about 5 g per patient per day). The amount of active ingredient that may be combined with the carrier materials to produce a single dosage form will vary depending upon the host treated and the particular mode of administration. For example, a formulation intended for the oral administration of humans may vary from about 5 to about 95% of the total composition. Dosage unit forms will generally contain between from about 1 mg to about 5000 mg of active ingredient. It will be understood, however, that the specific dose level for any particular patient will depend upon a variety of factors including the activity of the specific compound employed, the age, body weight, general health, sex, diet time of administration, route of administration, rate of excretion, drug combination and the severity of the particular disease undergoing therapy. The dosage effective amount of compounds according to the invention will vary depending upon factors including the particular compound, toxicity, and inhibitory activity, the condition treated, and whether the compound is administered alone or with other therapies.

As used herein, the term “administering” means providing a pharmaceutical agent or composition to a subject, and includes, but is not limited to, administering by a medical professional and self-administering. Administration of a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

The instant disclosure also includes kits, packages and multi-container units containing the herein described pharmaceutical compositions, active ingredients, and/or means for administering the same for use in the prevention and treatment of diseases and other conditions in mammalian subjects.

### FIGURES

The invention is further described by the following figures. These are not intended to limit the scope of the invention, but represent preferred embodiments of aspects of the invention provided for greater illustration of the invention described herein.

### BRIEF DESCRIPTION OF THE FIGURES

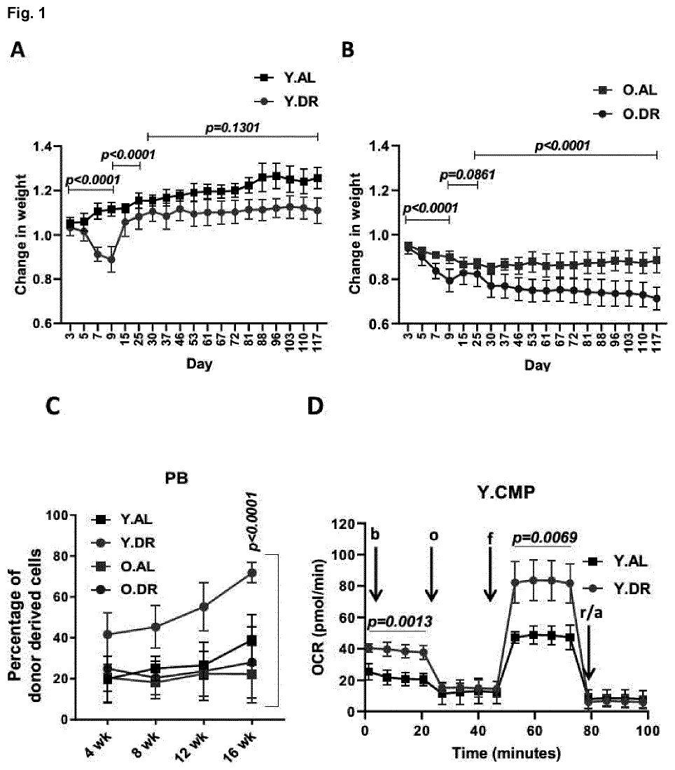

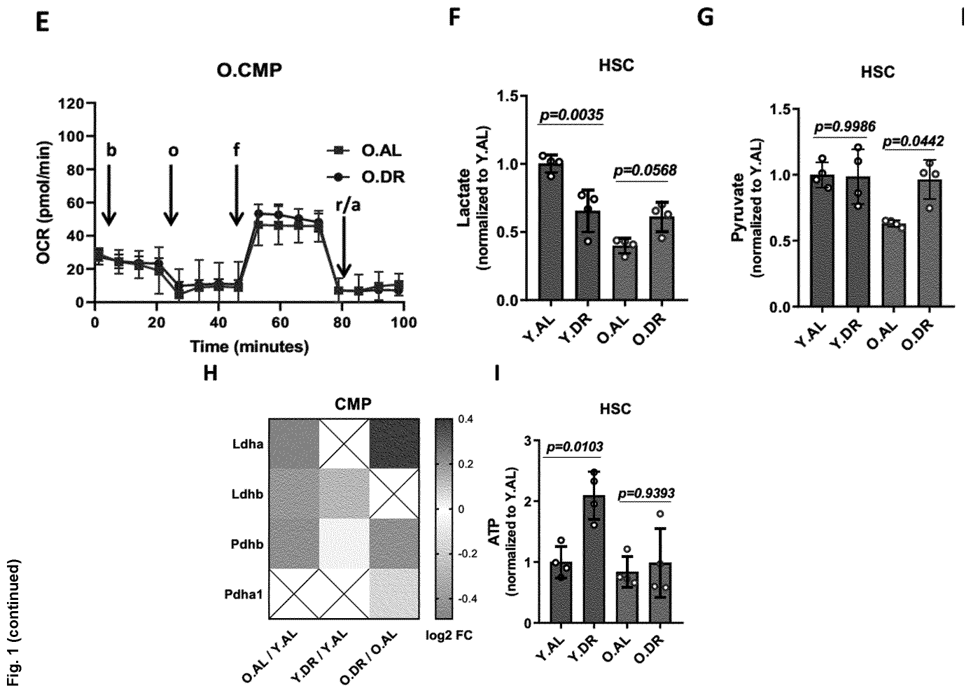

FIG. 1: Aging abrogates functional and metabolic plasticity of HSPCs in response to changes in nutrient availability.

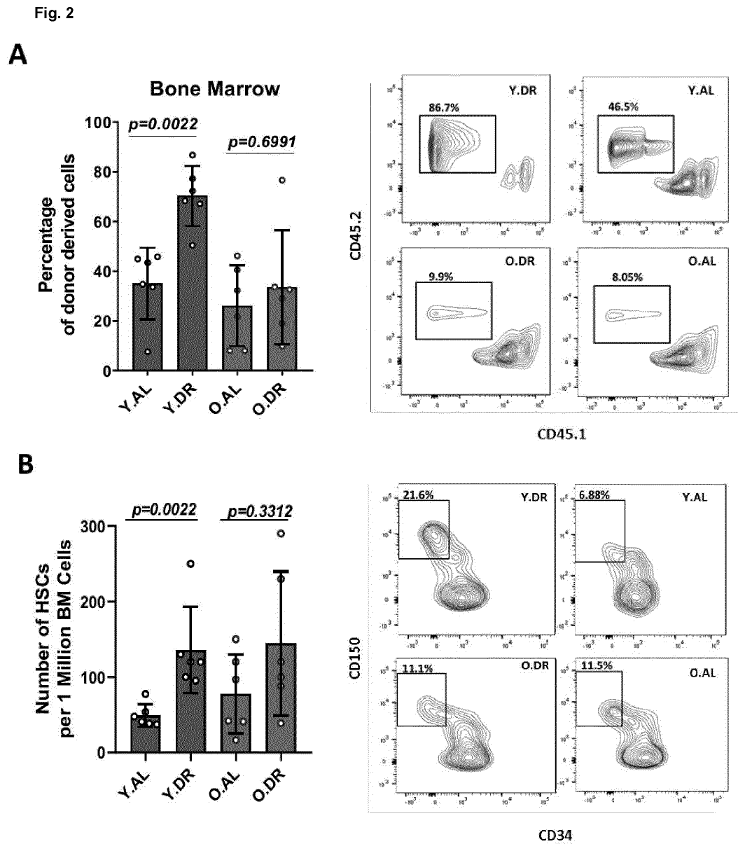

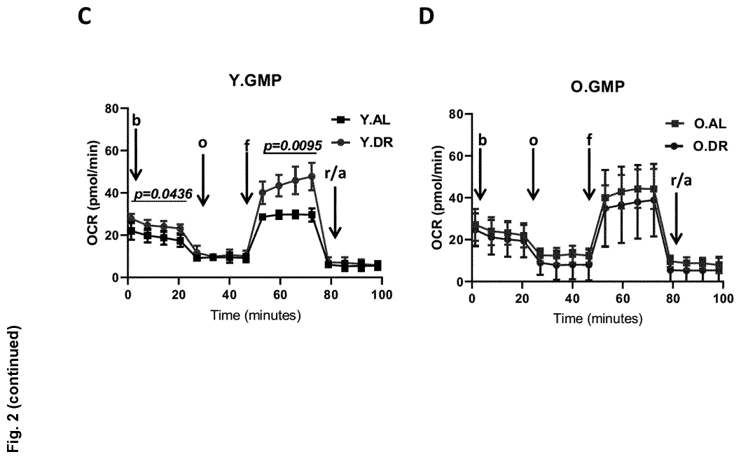

FIG. 2: Functional and metabolic adaptation of HSPCs in response to DR in old vs. young mice.

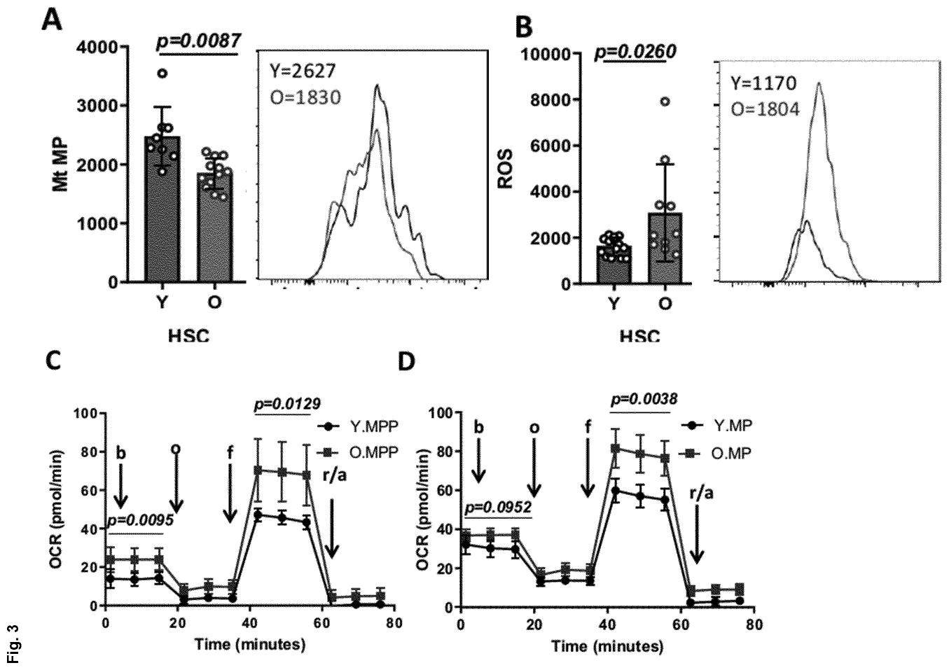

FIG. 3: Mitochondrial dysfunction in HSPCs of old vs. young mice.

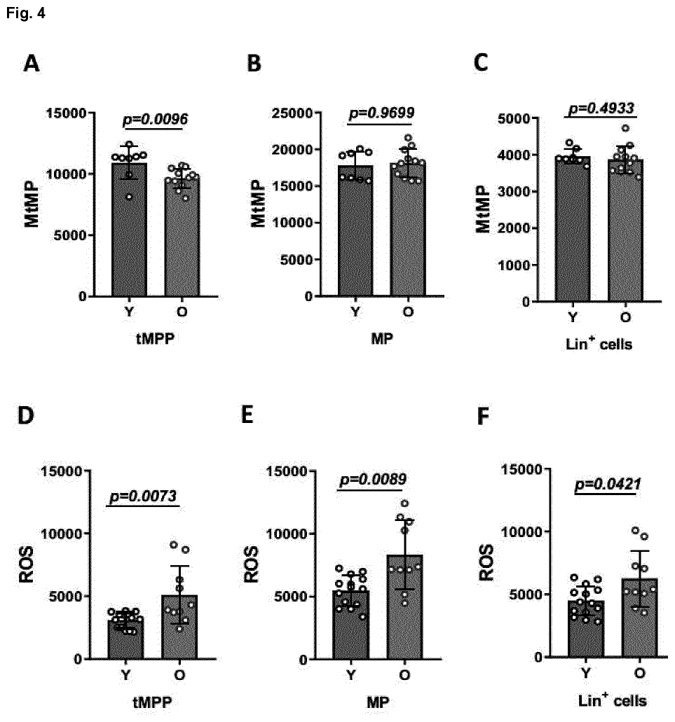

FIG. 4: Mitochondrial dysfunction in HSPCs from old vs. young mice.

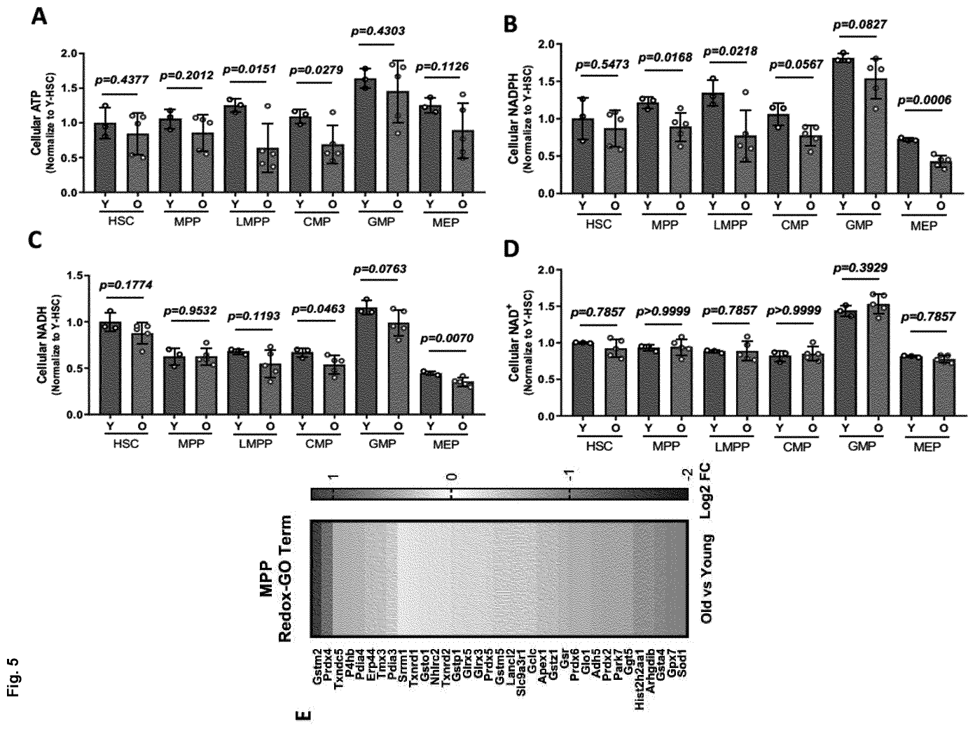

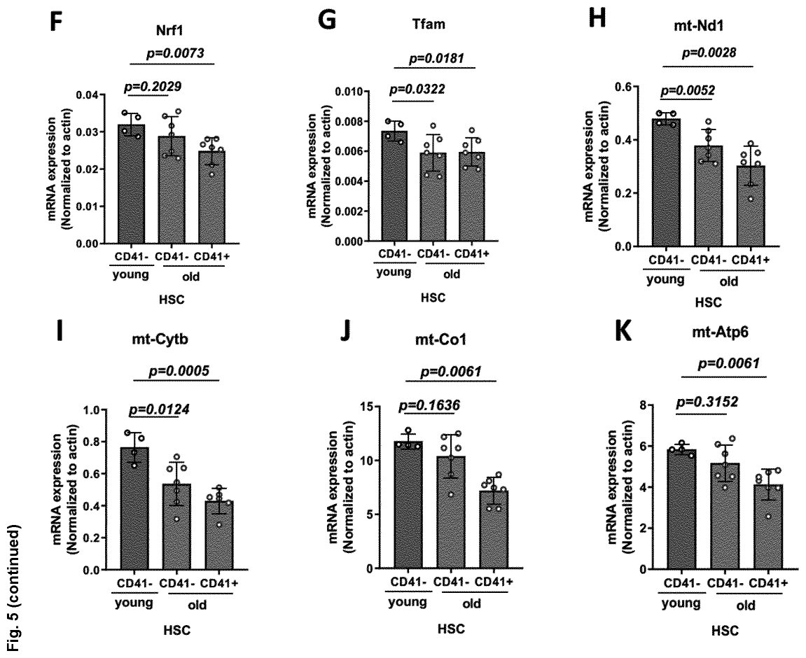

FIG. 5: Aging-associated disturbances in ATP and NAD(P) levels, redox homeostasis, and nuclear/mitochondrial encoded mitochondrial genes.

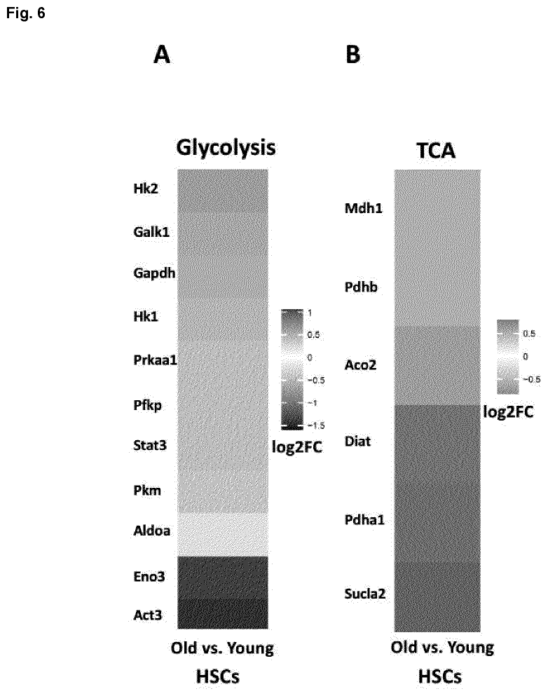

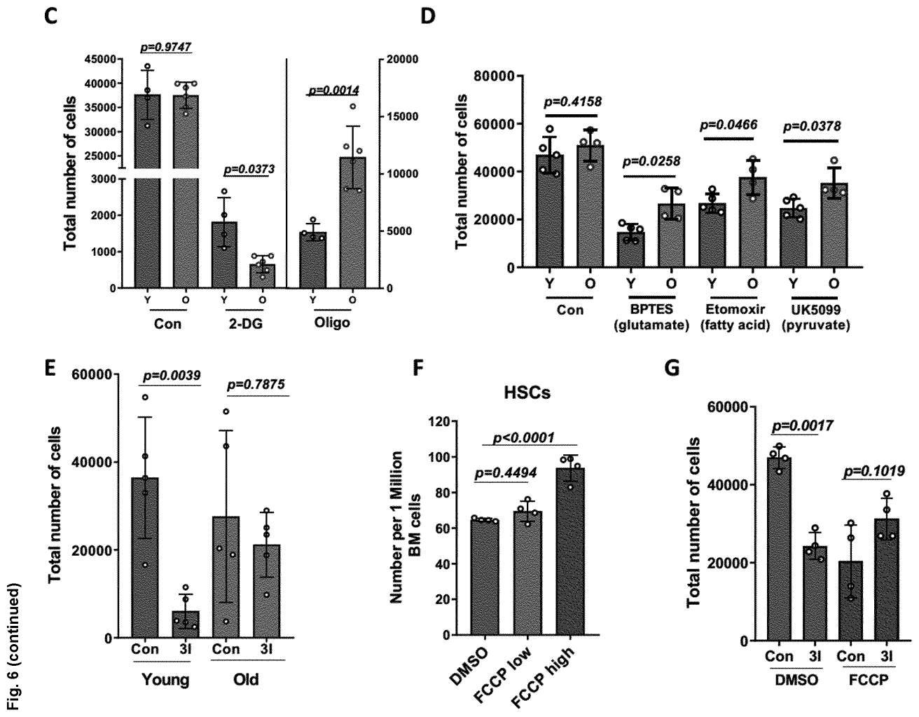

FIG. 6: Attenuated mitochondrial stress response in HSCs of aged mice and of mitotoxin-exposed young mice.

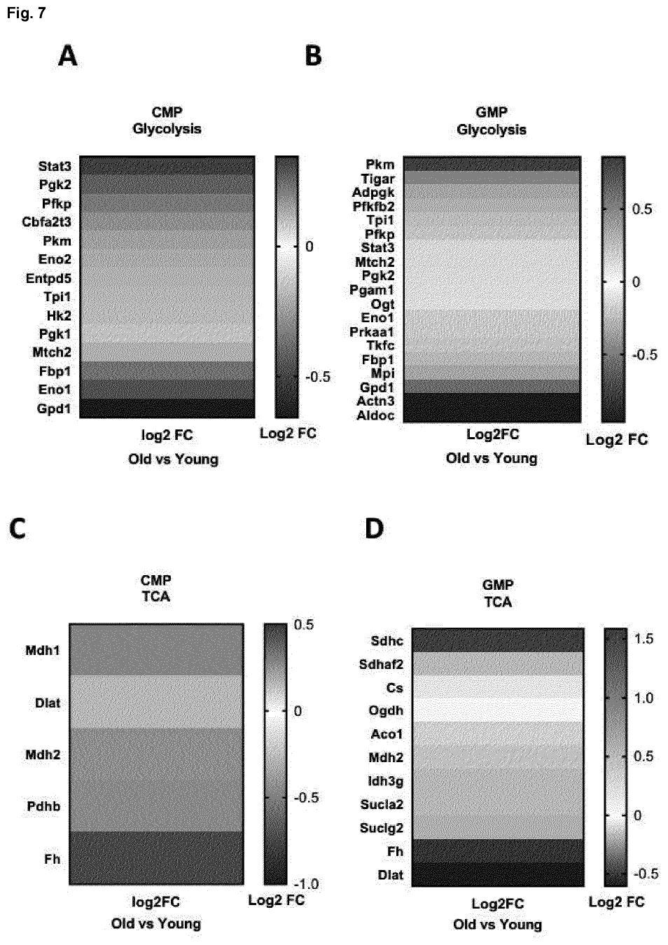

FIG. 7: Aging-associated alterations in the expression of proteins involved in glycolysis and TCA cycle in HSPCs.

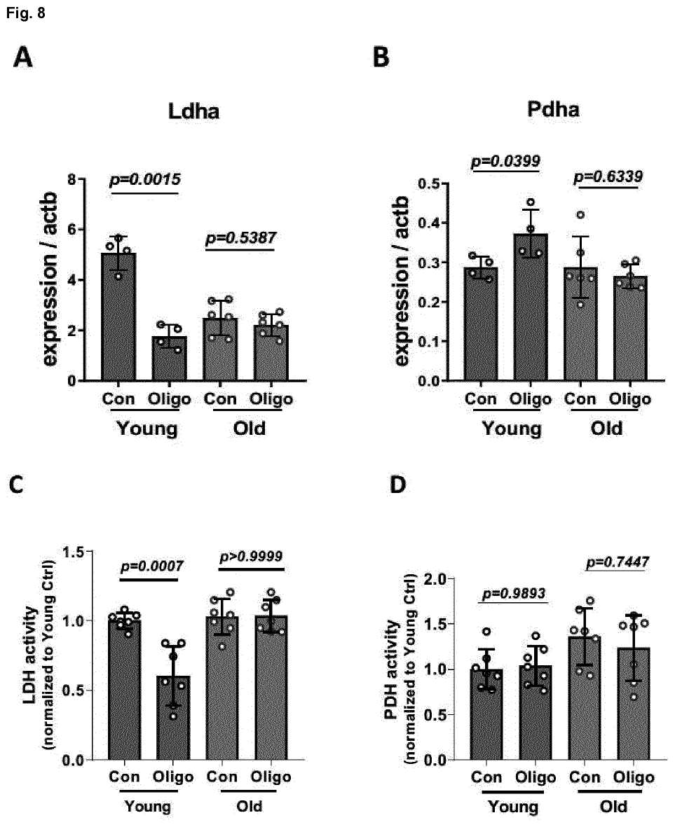

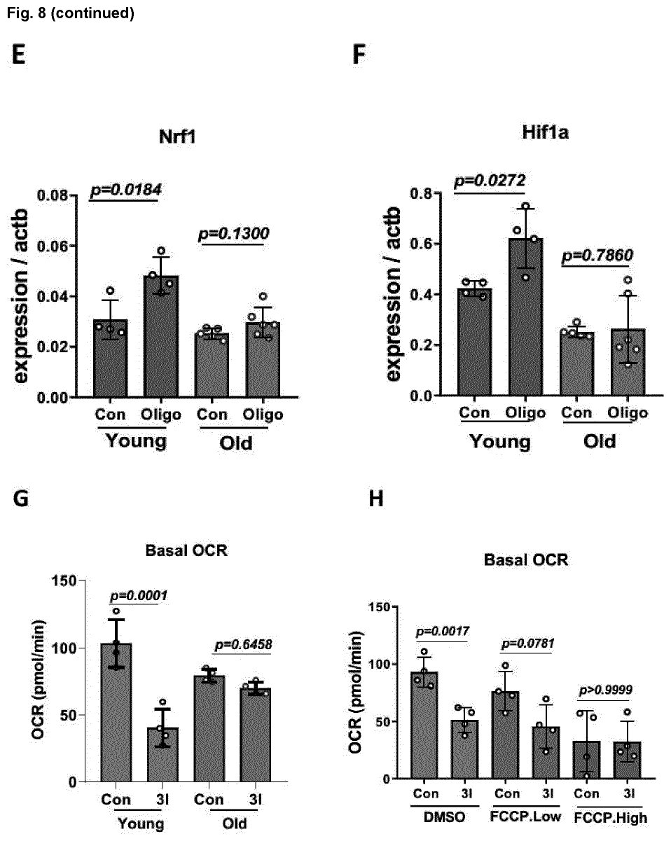

FIG. 8: Aging-associated and chemical-induced mitochondrial dysfunction abrogate metabolic plasticity in HSPCs.

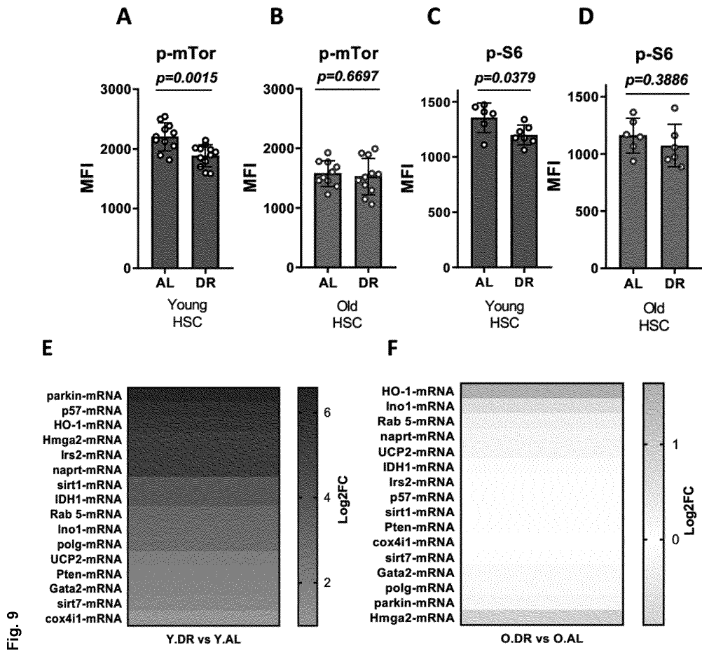

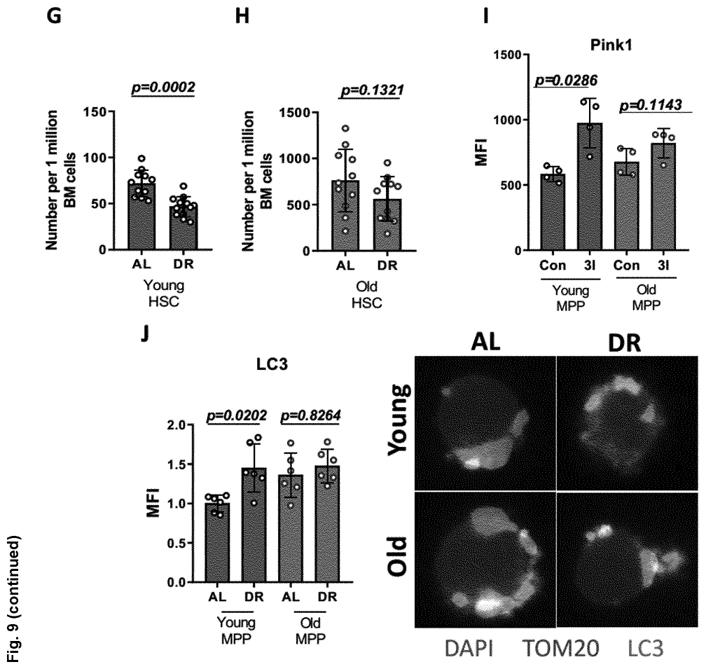

FIG. 9: Nutrient sensing, transcriptional stress responses, and the induction of clearance pathways are impaired in aged HSPCs.

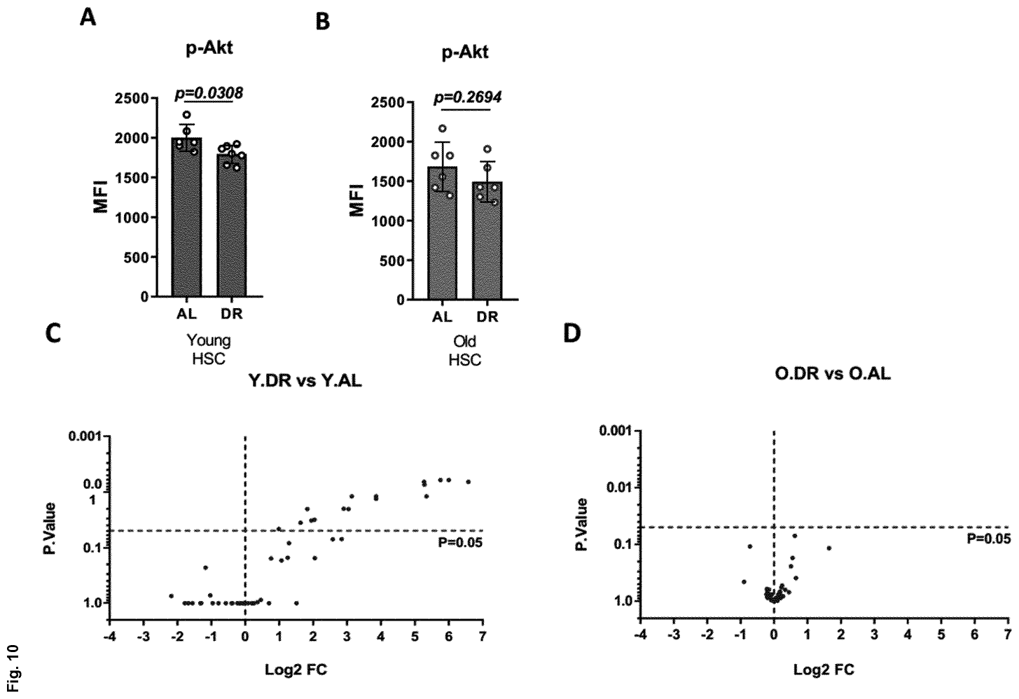

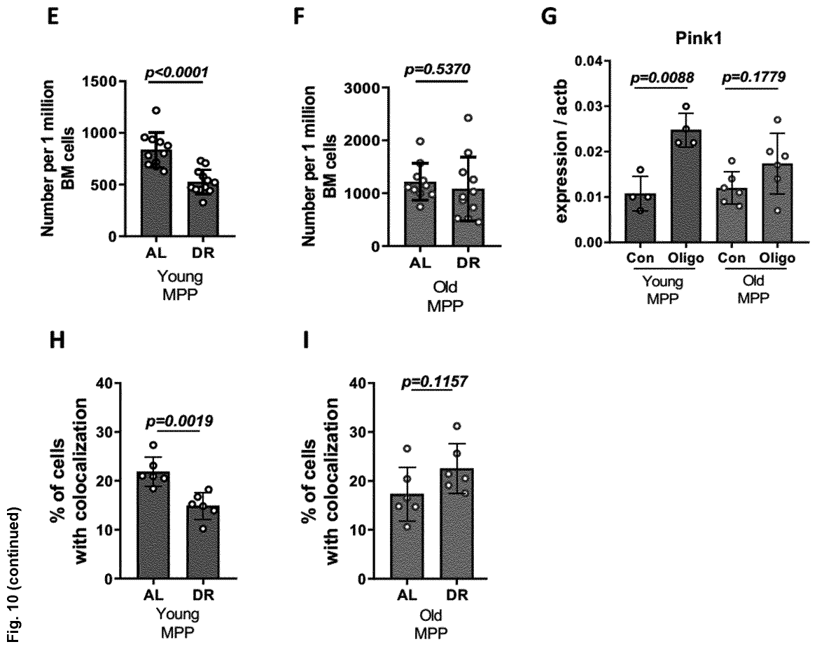

FIG. 10: Metabolic stress response in HSPCs from young and old mice in response to DR vs. AL diet.

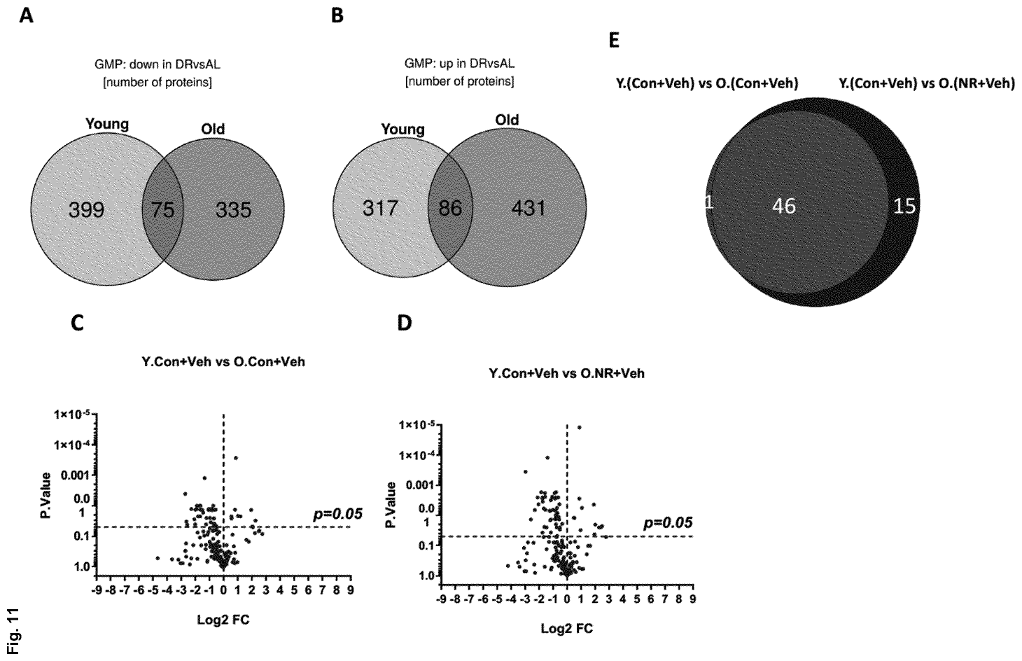

FIG. 11: DR induces age-specific changes in proteome of CMP and GMP. The restoration of response to metabolic stress in old HPSCs occurs after NR supplementation.

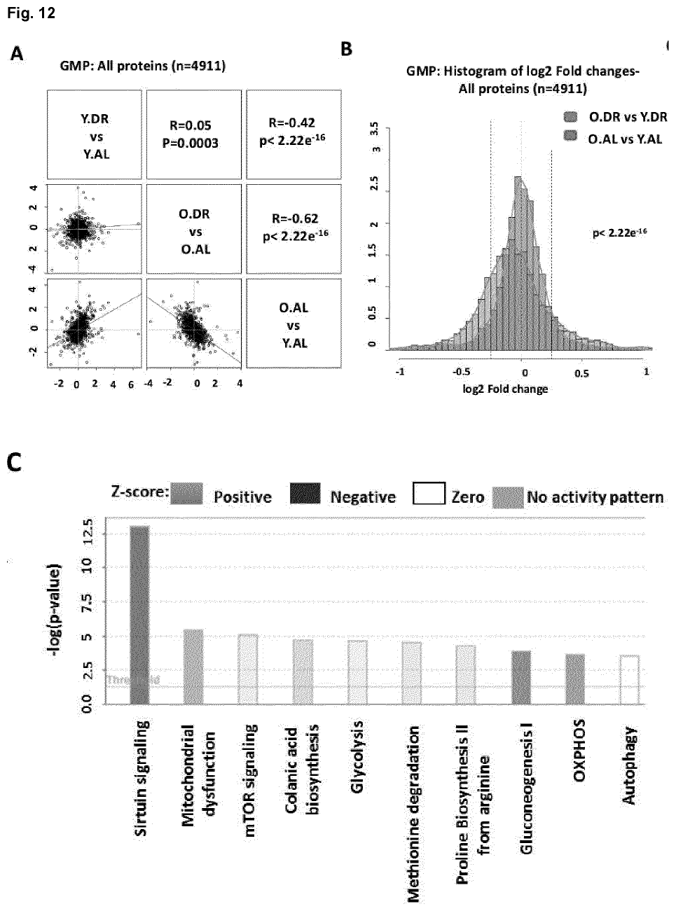

FIG. 12: DR ameliorates age-related changes in the proteome of HSPCs but NR supplementation is required to reinstall the responsiveness of aged HSPCs to metabolic stress.

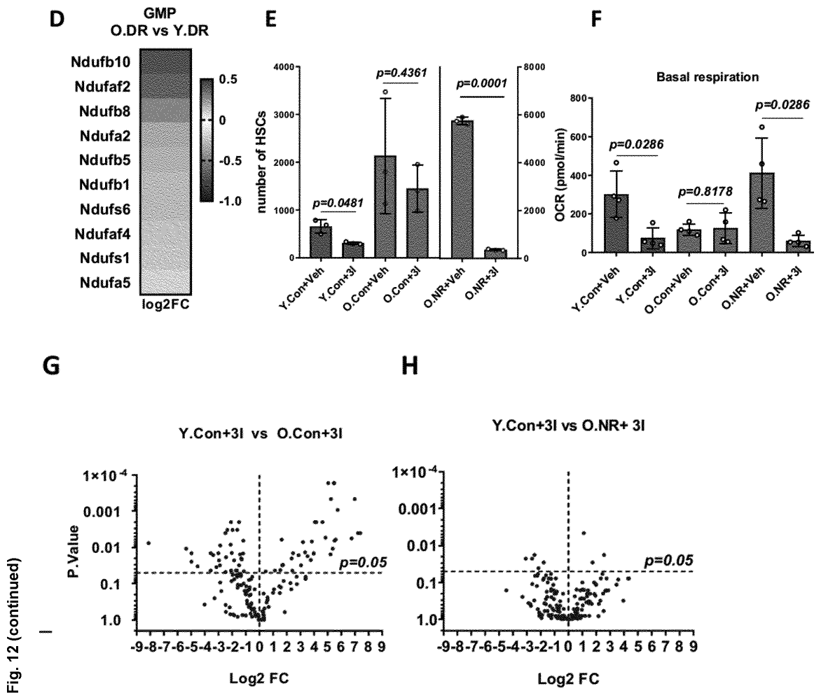

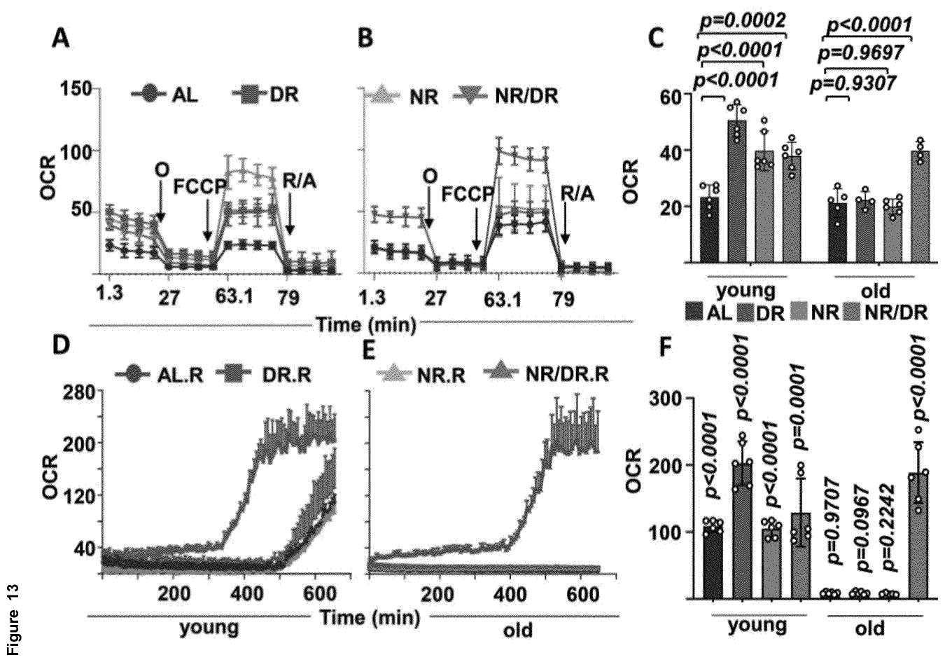

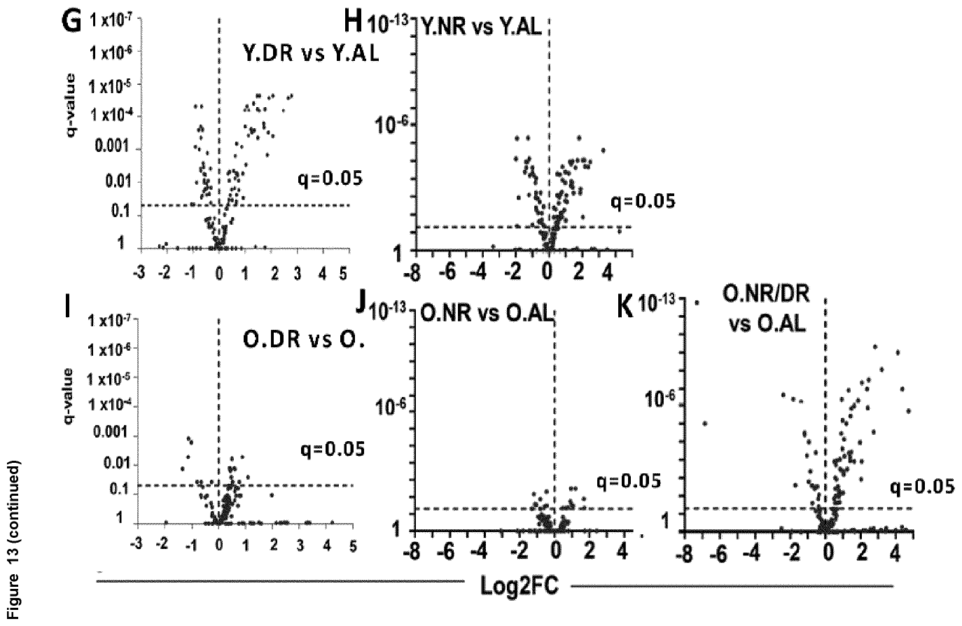

FIG. 13: NAD-precursor (NR) supplementation rescues mitochondrial metabolic plasticity and transcriptional stress responses in DR-exposed, old mice.

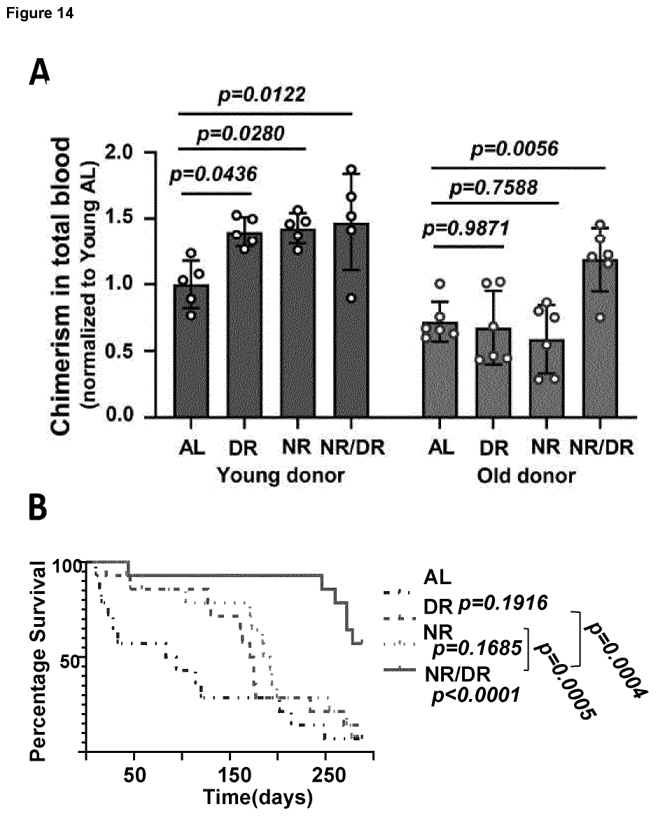

FIG. 14: NR/DR cotreatment improves repopulation capacity of HSC and lifespan of old mice.

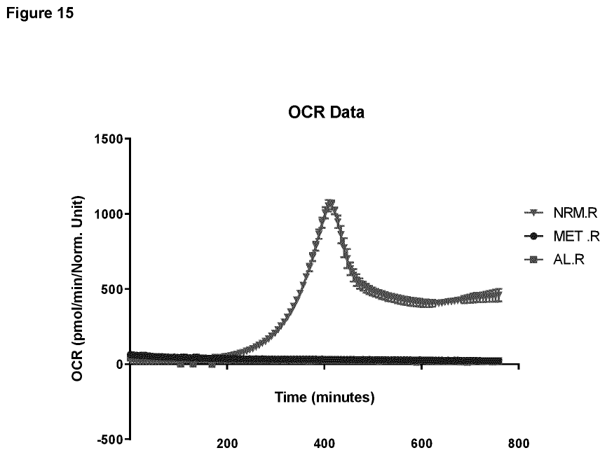

FIG. 15: Cotreatment of NR synergizes with the subclass of mitochondria-stress inducing of CRMs (such as Metformin) in old mice.

### DETAILED DESCRIPTION OF THE FIGURES

FIG. 1. (A,B) Young (2-3 months) and old (22-24 months) female mice (C57Bl/6j) were single caged and food intake was determined for 1 week, followed by exposure to dietary restriction (DR=70% of AL food intake) or a continuation of Ad libitum (AL) diet. The line graphs show percentages bodyweight change compared to the starting weight at the indicated time points after initiation of DR or under continuation of AL feeding for (A) young mice (n=5 for AL diet, n=10 for DR) and (B) old mice (n=5 for AL diet, n=10 for DR). The data passed Shapiro-Wilk normality test and p-values were calculated by paired t-test, each comparing change in weights between the indicated 2 time points of the DR group. Error bars depict SD.

(C) Peripheral blood chimerism of young (2-3 months old) recipient mice that were transplanted with 200 HSCs from young (Y) or old (O) donor mice that were exposed to DR or AL diet (donors were from the experiment shown in FIG. 1A,B). Test HSCs were transplanted together with 1×10⁶ competitor total bone marrow cells from 6 month old mice (Ly5.1). N=10 recipients per group, n=5-10 donors per group. P-values were calculated for the 16-week time point using 2-way ANOVA with Tukey's multiple comparisons test, after passing the Shapiro-Wilk normality test. Error bars depict SD.

(D,E) Seahorse analysis on 80,000 CMPs of (D) young (3 months) and (E) old (23 months), female mice exposed to 2 weeks AL diet or DR (n=4 mice per group). The graph shows the oxygen consumption rate at basal level (b), after oligomycin injection (o), after FCCP injection (f), and after injection of rotenone/antimycin (r/a). The curves depict the average respiration at the indicated conditions. Data were normally distributed (Shapiro-Wilk test) and p-values were calculated on average data of 4 timepoints per mouse per condition using unpaired t-test with Welch's correction. Error bars depict SD.

(F,G) HSCs (CD150+CD34⁻Flt3⁻LSK) were freshly isolated from 2-weeks DR or AL diet exposed, young mice (2-3 months=Y.AL or Y.DR) and from 2-weeks DR or AL diet exposed, old mice (22-24 months=O.AL or O.DR). HSCs were analyzed for (F) Lactate and (G) Pyruvate. The histograms depict fold-changes scaled to young, AL-fed mice (Y.AL) set to 1. N=4 mice per group. Data are normally distributed (Shapiro-Wilk test). P-values were calculated by two-way ANOVA with Tukey's multiple comparison tests. Error bars depict SD.

(H) The heat map shows the protein expression of Ldha, Ldhb, Pdhb, and Pdha1 in freshly isolated CMPs from young and old mice, 2 weeks after exposure to DR or AL diet (n=5/group). Color code indicates log 2-fold up- or downregulation of significantly regulated proteins (q-value <0.05). Red is for upregulation, blue for down-regulation, and the X signs refer to non-significant regulation.

(I) ATP levels in freshly isolated HSCs from the same mice as in (F,G). The histogram shows fold-changes scaled to young, AL-fed mice (Y.AL) set to 1. N=4 mice per group. Data are normally distributed (Shapiro-Wilk test). P-values were calculated by two-way ANOVA with Tukey's multiple comparison tests. Error bars depict SD.

FIG. 2. (A,B) Young recipient mice were transplanted with 200 HSCs from young (Y) or old (O) donor mice that were exposed to DR or AL diet (same experiment as shown in FIG. 1C). The histograms and representative FACS blots show (A) the chimerism in total bone marrow and (B) the frequency of HSCs (CD150+CD34-LSK) in donor-derived total bone marrow cells at 16 weeks after transplantation. (A,B) Representative FACS plots are shown on the right, in (B) it show percentages of HSCs in the LSK gate. The Mann Whitney test was used to calculate p-values since the data did not pass the Shapiro-Wilk normality test. Error bars depict SD.

(C,D) Seahorse analysis was done on 80,000 GMPs of (C) young (3 months) and (D) old mice (23 months) exposed to 2 weeks AL or DR (n=4 mice each). The data passed Shapiro-Wilk normality test. P-values in were calculated on average data of 4 timepoints per mouse per condition using unpaired t-test with Welch's correction. Error bars depict SD.

FIG. 3. (A) Total Bone marrow cells from young mice (5-6 months, n=8) and old mice (28-30 months, n=12) were stained with HSC markers, followed by Rhodamine123 staining for mitochondrial membrane potential (MtMP) analysis. The staining intensity (geometric mean) was analyzed in HSCs (CD150+CD34-LSK) by FACS. Representative FACS profiles are shown on the right. Data are normally distributed (Shapiro-Wilk test). P-value was calculated by t-test with Welch's correction. Error bars depict SD.

(B) Total Bone marrows from young mice (2-3 months, n=14) and old mice (27-30 months, n=10) were stained with HSC markers, followed by CellROX green staining for ROS analysis. The staining intensity (geometric mean) was analyzed in HSCs (CD150+CD34-LSK) by FACS. Representative FACS profiles are shown on the right. Data did not show normal distribution (Shapiro-Wilk test). P-value was calculated by Mann Whitney test. Error bars depict SD.

(C,D) Seahorse analysis was carried out on 80,000 (C) MPPs (CD34*LSK) and (D) myeloid progenitors (MPs: lin-c-Kit*Sca1-, including CMP, GMP, and MEP). Cells were freshly isolated from young (4-5 months) and old mice (31 months). The graph shows the oxygen consumption rate at basal level (b), after oligomycin injection (o), FCCP injection (f), and after rotenone/antimycin injection (a/r). Data were normally distributed (Shapiro-Wilk test), p-values were calculated on average data of 4 timepoints per condition per mouse using unpaired t-test with Welch's correction. Error bars depict SD.

FIG. 4. (A-C) The mitochondrial membrane potential (MtMP, same experiment as shown in FIG. 3A) was determined in the following subpopulations of total bone marrow cells from young mice (5-6 months, n=8) and old mice (28-30 months, n=12): (A) total MPPs (=tMPP: CD34*LSKs), (B) myeloid progenitor cells (=MP: Lin⁻c-Kit⁺), and (C) Lin⁺ cells. (A-C) Normality of data was assessed by Shapiro-Wilk test, and p-values were calculated by (A,B) Mann Whitney test or (C) unpaired t-test with Welch's correction. Error bars depict SD.

(D-F) Levels of reactive oxygen species (ROS, same experiment as shown in FIG. 3B) were determined in the following subpopulations of total bone marrow cells from young mice (2-3 months, n=14) and old mice (27-30 months, n=10): (D) total MPP (=tMPP, CD34⁺LSKs), (E) myeloid progenitor (=MP, Lin⁻c-Kit⁺), and (F) Lin⁺ cells. (D-F) Normality of data was assessed by Shapiro-Wilk test. (D) Data are not normally distributed, p-values were calculated by Mann Whitney test, (E,F) data are normally distributed, p-values were calculated by unpaired t-test with Welch's correction. Error bars depict SD.

FIG. 5. (A-D) Freshly isolated HSCs (CD150+CD34⁻Flt3⁻LSK), MPPs (CD34⁺Flt3⁻LSK), LMPPs (CD150-Flt3+LSK), CMPs (Lin⁻c-Kit⁺Sca1⁻CD34+FcγR^{-/low}), GMPs (Lin⁻c-Kit⁺Sca1⁻CD34+FcγR⁺), MEPs (CD34-FcYR⁻Lin⁻c-Kit+) from young mice (6 months, n=3 pools of 6 mice per pool) and old mice (24 months, n=5 individual mice) were analyzed for (A) ATP, (B) NADPH, (C) NADH, and (D) NAD+ by LC-MS. (A-D) Data were normalized to the number of cells for each sample. For depiction in the histograms, each data set was scaled to the average value of HSCs from young, AL-fed mice (Y.AL) set to 1. Statistical analysis: normality of data was assessed by Shapiro-Wilk test. (A-C) data are normally distributed; p-values were calculated by unpaired t-test with Welch's correction. (D) Data are not normally distributed; p-values were calculated by Mann Whitney test. Error bars depict SD.

(E) The heat map shows redox proteins that were detectable in LC-MS/MS-proteome analysis and showed a significant difference in expression levels (q-value <0.05) in freshly isolated MPPs (CD34⁺Flt3⁻LSK) from old mice (30-36 months) versus young mice (6-8 months). Color coding indicates log 2-fold of upregulated (red) and downregulated (blue) proteins.

(F-K) Freshly isolated HSCs (CD150⁺CD34⁻Flt3⁻LSK) from young mice (4 months, n=4) and old mice (28-30 months, n=7) were analyzed for mRNA expression of target genes (relative to β-actin). For old mice HSCs were separated into CD41− and CD41+ HSCs. CD41+ HSCs were present at very low levels in young mice (<5%) and thus not isolated. (F,G) nuclear encoded, mitochondrial associated genes and (H-K) electron transport chain complex genes encoded by mitochondrial genome. Normality of data was assessed by Shapiro-Wilk test after Log 2 transformation. (F-1) Data are normally distributed and p-values were calculated by unpaired t-test with Welch's correction on Log 2-transformed data. (J,K) Data are not normally distributed and p-values were calculated by Mann Whitney test on Log 2-transformed data. Error bars depict SD.

FIG. 6. (A,B) The heat map shows (A) glycolysis-related proteins and (B) TCA-cycle-related proteins that were detectable in LC-MS/MS-proteome analysis and showed a significant difference in expression levels (q-value <0.05) in freshly isolated HSCs (CD150 from old mice (24 months) versus young mice (6-8 months). Color coding indicates log 2-fold of upregulated (red) and downregulated (blue) proteins. N=4 pools of in total 36 young mice and n=4 pools of in total 10 old mice.

(C-E) One-thousand-five-hundred, freshly isolated HSCs (CD150⁺CD34⁻Flt3⁻LSK) from young or old mice were cultured per well (96-well plate). (C) HSCs were exposed to 2-DG (3 mM), Oligomycin (=Oligo, 1.8 nM), or DMSO as control (=Con). Total cell numbers per well were determined by FACS after 5 days. Young mice (4 months, n=4) and old mice (24 months, n=6). Data are normally distributed (Shapiro-Wilk test). P-values were calculated by t-test with Welch's correction. Error bars depict SD. (D) HSCs were cultured with inhibitors that block the mitochondrial uptake of glutamate (BPTES, 9 μM), fatty acids (Etomoxir, 9 μM), or pyruvate (UK5099, 9 μM). Total cell numbers were counted by FACS after 5 days. Young mice (3-4 months, n=5) and old mice (26 months, n=4). Data are normally distributed (Shapiro-Wilk test). P-values were calculated by unpaired t-test with Welch's correction. (E) HSCs were exposed to a combination of 3 inhibitors (3 inhibitors=3I, same as in D) at a concentration of 6 μM (each) or to DMSO (Con). Young mice (2-3 months, n=5) and old mice (30-31 months, n=5). Data are normally distributed (Shapiro-Wilk test). P-values were calculated using 2-way ANOVA with Tukey's multiple comparisons test. Error bars depict SD.

(F,G) Young, 3-4 month old mice were treated within one week with two intraperitoneal injections of FCCP on day-1 and day-4 (high-dose: 4.8 mg/Kg or low-dose: 1.2 mg/Kg) or with vehicle control (10% DMSO) (n=4 mice per group). One week after the start of the treatment (day-7), total bone marrow cells were analyzed and used for culture experiments. (F) The histogram shows the frequency of HSCs (CD150⁺CD34⁻Flt3⁻LSK) per million total bone marrow cells directly after isolation in the indicated groups of mice. (G) 2,500 freshly isolated HSCs from vehicle-treated of high-dose FCCP-treated animals were cultured and exposed to 3I-treatment (same concentration as in FIG. 6E) or to vehicle-treatment (Con). The histogram depicts the total cell numbers at 4 days after culture initiation. (F,G) Data are normally distributed (Shapiro-Wilk test) and p-values were calculated using one-way ANOVA (F) and two-way ANOVA (G) with Tukey's multiple comparisons test. Error bars depict SD.

FIG. 7. (A-D) The heat maps show (A,B) glycolysis-related proteins or (C,D) TCA-cycle related proteins that were detectable in LC-MS/MS-proteome analysis and showed a significant difference in expression levels (q-value <0.05) in freshly isolated in (A,C) CMPs (Lin⁻c-Kit⁺Sca1⁻CD34⁺FcγR⁻/^{l}ow) and (B,D) GMPs (Lin⁻c-Kit⁺Sca1⁻CD34⁺FcγR⁺) from young (3-4 months, n=4-5) and old mice (25-26 months, n=4-5) Color coding indicates log 2-fold of upregulated (red) and downregulated (blue) proteins.

FIG. 8. (A,B) Freshly isolated HSCs (CD150⁺CD34⁻Flt3⁻LSK) from young mice (2-3 months, n=4) and old mice (27-30 months, n=6) were exposed to oligomycin-treatment (0.6 μM) or vehicle control (0.1% DMSO) for 3 hours in culture (7000 HSCs per well of a 96-well plate). Gene expressions (relative to β-actin) of (A) Ldha, (B) Pdha were analyzed by qPCR. Log 2-transformed data showed normal distribution (Shapiro-Wilk test). P-values were calculated on transformed data by unpaired t-test with Welch's correction. Error bars depict SD.

(C,D) Freshly isolated HSCs (CD150⁺CD34⁻Flt3⁻LSK) from young mice (3-4 months, n=7) and old mice (24-25 months, n=7) were cultured overnight (7,000 HSCs per well of a 96-well plate). Eighteen hours later, HSCs were exposed to oligomycin-treatment (2.4 nM) or vehicle control (0.04% DMSO). After 20h treatment, 1,000 cells from the cultures were isolated by FACS to analyze the activity of (C) LDH and (D) PDH. The histograms depict the data scaled to the average of young, control-treated HSCs set to 1. Data are normally distributed (Shapiro-Wilk test). P-values were calculated by two-way ANOVA with Tukey's multiple comparison tests. Error bars depict SD.

(E,F) The same HSC cultures that were described in FIG. 8A were used to determine the mRNA expression levels of (E) Nrf1 and (F) Hif1a relative to β-actin by qPCR. N=4 for young groups, n=6 for old groups. One outlier (Grubbs test) was removed for the control groups). Log 2 transformed data showed normal distribution and p-values were calculated on the transformed data by unpaired t-test with Welch's correction. Error bars depict SD.

(G) Freshly isolated CMPs (Lin⁻c-Kit⁺Sca1⁻CD34⁺FcγR^{-/low}), from young (3 months, n=4) and old mice (24 months, n=4) were cultured and exposed for 12h to 3 inhibitors of mitochondrial nutrient uptake (3I=BPTES=6 uM, Etomoxir=6 uM, UK5099=6 uM) or DMSO control (Con). Afterwards the basal oxygen consumption rate (OCR) of the indicated groups was measured by Seahorse. Statistical analysis: The data were normally distributed (Shapiro-Wilk) and p-values were calculated by 2-way ANOVA with Tukey's multiple comparisons test. Error bars depict SD.

(H) Young, 3-4-month old mice were treated one week with two intraperitoneal injections of FCCP/week (high-dose: 4.8 mg/Kg or low-dose: 1.2 mg/Kg) or with vehicle control (10% DMSO) (n=4 mice per group). One week after the start of the treatment, CMPs were freshly isolated and exposed for 12h in culture to a combination of 3 inhibitors of mitochondrial nutrient uptake (31: 6 μM of each BPTES, Etomoxir, and UK5099) or DMSO control (Con). Statistical analysis: The data were normally distributed (Shapiro-Wilk) and p-values were calculated by 2-way ANOVA with Tukey's multiple comparisons test. Error bars depict SD.

FIG. 9 (A-H) Young mice (2-3 months) and old mice (22-24 months) were exposed for 2 weeks to DR (Y.DR and O.DR, respectively) or continuation of AL diet (Y.AL and O.AL, respectively), followed by the analysis of bone marrow cells:

(A-D) Freshly isolated bone marrow cells were stained for HSCs (CD150⁺CD34⁻Flt3⁻LSK) along with fluorescence conjugated, antibodies against (A,B) phosphorylated-mTOR (p-mTor) or (C,D) phosphorylated ribosomal protein S6 (p-S6). Cells were analyzed by FACS. The geometric means of fluorescence intensities (MFIs) in the HSC population of individual mice is shown in (A,B) for p-mTOR: (n=11 for Y.AL, n=12 for Y.DR, n=10 for O.AL, n=11 for O.DR) and in (C,D) for p-S6 (n=6 for Y.AL and O.AL, n=7 for Y.DR and O.DR). Statistical analysis: data are normally distributed (Shapiro-Wilk test) and p-values were calculated by unpaired t-test with Welch's correction. Error bars depict SD.

(E,F) The mRNA of freshly isolated MPPs was analysed by a customized NanoString platform detecting 52 genes that have a known role in metabolic stress responses. (E) The heatmap shows significantly regulated genes in MPPs from young, DR-exposed mice compared to MPPs from young, AL-fed mice (p-value <0.05 after Benjamini-Yekutieli adjustment using nSolver v.4 software from Nanostring Techn.). (F) shows the expression profile of the same set of genes in MPPs from old, DR-exposed mice in comparison to MPPs from old, AL-fed mice. Note that all stress response genes that significantly reacted to DR in MPPs from young mice showed increased expression levels compared to AL-fed young mice (red color code). In contrast, none of the stress response genes was significantly regulated in response to DR in MPPs from old mice.

(G,H) FACS analysis of freshly isolated bone marrow was used to determine the total HSC numbers (CD150⁺CD34⁻Flt3⁻LSK) per million total bone marrow cells in (G) young and (H) old mice that were exposed to 2 weeks DR or AL diet. N=11 for Y.AL, n=12 for Y.DR, n=11 for O.AL, n=11 for O.DR. Statistical analysis: data are normally distributed (Shapiro-Wilk test) and p-values were calculated by the unpaired t-test with Welch's correction. Error bars depict SD.

(I) freshly isolated MPPs (CD34⁺Flt3⁻LSK) from young and old, AL-fed mice (n=4/group) were treated for two days in culture with a combination of 3 inhibitors of mitochondrial nutrient uptake (3I: 6 μM of each BPTES, Etomoxir, and UK5099) or DMSO (0.4%). Cells were then stained with a conjugated fluorescence-antibody against PINK1. The histogram shows the geometric means of the fluorescence intensity (MFIs) of PINK1 staining in the HSC population of individual mice. Statistical analysis: The data were not normally distributed (Shapiro-Wilk test) and the Mann-Whitney test was used for the calculation of p-values.

(J) Young mice (2-3 months) and old mice (22-24 months) were exposed for 1 week to DR or continuation of AL diet. Afterwards, freshly isolated MPPs (CD34⁺Flt3⁻LSK) were co-stained for the mitochondrial protein TOM20 and the autophagy-related protein LC3 and analyzed under the microscope. Quantification of mean fluorescence intensity (MFI) of LC3 protein expression was conducted by Image J. N=6 mice per group. The histograms depict data that were scaled to the average of staining intensity values in young AL-fed mice set to 1. Representative images of staining of TOM20 (red), LC3 (green) and DAPI (blue) are shown on the right. Data are normally distributed (Shapiro-Wilk test) and p-values were calculated by two-way ANOVA with Tukey's multiple comparison tests. Error bars depict SD.

FIG. 10. (A,B) Volcano plots on the indicated groups of the NanoString analysis from FIG. 9E,F. Horizontal, dashed line indicates threshold for genes that are significantly regulated in (A) young DR-exposed mice (Y.DR) versus young AL-fed mice (Y.AL) and in (B) old DR-exposed mice (O.DR) versus old AL-fed mice (O.AL). Note that 16 stress response genes were significantly induced in response to DR in young mice, while none of the tested 52 stress response genes was regulated in response to DR in old mice.

(C,D) The levels of phosphorylated AKT (p-AKT) in HSCs (CD150⁺CD34⁻Flt3⁻LSK) were analyzed by FACS on the same mice as in FIG. 9C-D. Data are normally distributed (Shapiro-Wilk test) and p-values were calculated by t-test with Welch's correction. Error bars depict SD.

(E,F) FACS analysis of freshly isolated bone marrow cells on the total numbers of MPPs (CD34⁺Flt3⁻LSK) per million total bone marrow cells in (E) young and (F) old mice that were exposed to 2 weeks DR or AL diet. N=11 for Y.AL, n=12 for Y.DR, n=10 for O.AL, n=11 for O.DR. Statistical analysis: data are normally distributed (Shapiro-Wilk test) and p-values were calculated by the unpaired t-test with Welch's correction. Error bars depict SD.

(G) Freshly isolated MPPs (CD34⁺Flt3⁻LSK) from young mice (2-3 months, n=4) and old mice (27-30 months, n=6) were exposed to oligomycin (0.6 μM) or vehicle control (0.1% DMSO) for 3 hours in culture. Gene expression of Pink1 (relative to E-actin) was measured by qPCR. The transformed data are normally distributed (Shapiro-Wilk test) and p-values were calculated on transformed data by unpaired t-test with Welch's correction. Error bars depict SD.

(H,I) Colocalization of the autophagy protein LC3 and the mitochondrial protein TOM20 was analyzed by microscopy in freshly isolated MPPs (CD34⁺Flt3⁻LSK) from young and old mice that were exposed for 1 week to DR or continuation of AL diet (the same experiment as in FIG. 9J). Histograms depict the percentage of cells with LC3/TOM20 colocalization in the total number of MPPs imaged from each mouse. Data are normally distributed (Shapiro-Wilk test) and p-values were calculated by the unpaired t-test with Welch's correction. Error bars depict SD.

FIG. 11. (A,B) The Venn diagram shows the overlap between significantly (A) down-regulated and (B) up-regulated proteins that were detectable in LC-MS/MS-proteome analysis (q-value <0.05) in freshly isolated GMPs (Lin⁻c-Kit*Sca1⁻CD34⁺FcγR⁺) from young (3-4 months, n=4-5) and old mice (25-26 months, n=4-5) that were exposed for 2 weeks to DR or continuation of ad libitum food. (A,B) shows the comparison of Y.DR vs. Y.AL and O.DR vs. O.AL

(C,D) NanoString analysis on the expression of stress response genes was carried out on 10,000 MPPs (CD34⁺Flt3⁻LSK) from young (3-4 months, n=4) and old mice (22-24 months, n=4). MPPs were pre-treated in culture with 1 mM NR or DMSO (Con) for 12h followed by treatment with 31 or Vehicle (Veh: 0.4% DMSO) for 12h under continuation of NR or Veh treatment. (C,D) Volcano plots for the NanoString analysis. Horizontal, dashed line indicates threshold for genes that are significantly regulated in (C) MPPs from young mice and old mice that were exposed in culture to control treatments (Y.Con+Veh) versus (O.Con+Veh) and in (D) MPPs from young mice were exposed to control treatment versus MPPs from old mice that were exposed to NR followed by vehicle treatment (=Y.Con+Veh versus O.NR+Veh).

(E) The Venn-Diagram shows the overlap of significantly regulated genes (p-value below 0.05) from analysis in FIG. 11C,D.

FIG. 12 (A) Scatter plot correlation matrix for proteome comparisons. Protein abundances in GMPs from mice of different age and DR vs. AL-diet status were compared. From these comparisons, the log 2 fold change values were used to infer correlation and thus possible similarity of regulation patterns. The diagonal denotes the age/diet group comparisons. Upper triangle: Spearman correlation between proteome comparisons. Lower triangle: Scatter plot of average log 2 fold changes (4-5 biological replicates per age/diet group). Red lines show a fitted linear model.

(B) Histogram of log 2 fold changes in O.DR vs. Y.DR (red) and O.AL vs. Y.AL (blue). Solid curves show probability density estimations. The change in differentially expressed proteins of both comparisons was calculated by Brown-Forsythe Test for difference of variance.

(C) Ingenuity Pathway Analysis (IPA) was carried out on proteome changes of GMPs from old (24 months, n=5) compared to young (3 months, n=5) mice exposed to 2 weeks DR (red histogram of log 2 fold changes in 7B). The graph shows the top-10 enriched pathways using a cut-off including all significantly, differently expressed proteins (q-value 0.05).

(D) The heat maps shows NADH-ubiquinone oxidoreductase subunits of mitochondrial membrane complex I (NDUFs) proteins that were included under the term “Sirtuin signaling” in the IPA analysis depicted in (C) and showed a significant difference in expression levels (q-value <0.05) in freshly isolated GMPs from old (24 months, n=5) and young (3 months, n=5) mice exposed to DR (O.DR vs. Y.DR).

(E) Freshly isolated HSCs from young mice (3-4 months) and old mice (22-24 months) were exposed to 1 mM Nicotine riboside (NR) or DMSO (con) for 2 days followed by treatment with a combination of 3 inhibitors of mitochondrial nutrient uptake (31: 6 μM of each BPTES, Etomoxir, and UK5099) or vehicle (0.4% DMSO=Veh) for another 2 days. The NR treatment was continued on das 3 and 4. The histogram shows the total number of the stem cell containing subpopulation (CD48−, Sca1+) of HSC-derived cultures from young (Y) and old (O) mice exposed to the indicated treatment regiment for 4 days: Y.Con+Veh, Y.Con+3I, O.Con+Veh, O.Con+3I, O.NR+Veh, O.NR+3I. N=3 HSC cultures derived from 3 individual mice per group. Data are normally distributed (Shapiro-Wilk test) and p-values were calculated by the paired t-test with Welch's correction. Error bars depict SD.

(F) Basal oxygen consumption rate in CMPs from young (3-5 months, n=4) and old mice (22-24 months, n=4). Cells were either pretreated with 1 mM Nicotine riboside (NR) or DMSO (Con) for 12 h. This was followed by treatment for 12h in vitro to combination of 3 inhibitors of mitochondrial nutrient uptake (by BPTES=6 uM, Etomoxir=6 uM, UK5099=6 uM) or DMSO as control. The data were not normally distributed (Shapiro-Wilk test) and the Mann-Whitney test was used for the calculation of p-values.

(G,H) NanoString analysis on the expression of stress response genes was carried out on 10,000 MPPs (CD34⁺Flt3⁻LSK) from young (3-4 months, n=4) and old mice (22-24 months, n=4). MPPs were pre-treated in vitro with 1 mM NR or DMSO (Con) for 12h followed by treatment with 31 or Vehicle (Veh: 0.4% DMSO) for 12h. The NR treatment was continued for the time window of 12-24h. (G, H) Volcano plots on the NanoString analysis of stress response genes. Horizontal, dashed line indicates threshold for genes that are significantly regulated in (G) 3I-exposed MPP from young mice (Y.Con+3I) versus 3I-exposed MPPs from old mice (O.Con+3I) and in (H) 3I-exposed MPPs from young mice (Y.Con+3I) versus 3I-exposed MPPs from old mice that were pre-treated for 12h with NR (O.NR+3I).

FIG. 13 (A-J) Young (2-3 month old) and old (22-24 month old) mice were exposed for 2 weeks to the indicated diet: ad libitum diet (AL), dietary restriction (DR=CRD), NAD-precursor supplementation (=NR), NR/DR—combination of NR plus DR.

(A-C) Respirometry analysis of the OCR of freshly-isolated CMPs in: (A) young and (B) old mice; (C) Quantification of OCR at basal level of the indicated groups as measured in (A,B, before oligomycin-application, marked by 0). N=4-6 mice per group. The Data were normally distributed (Shapiro-Wilk test) after log transformation. Error bars represent SD. P-values were calculated on average data of 4 time points using 2-way ANOVA with Tukey's multiple comparisons test on log-transformed data.

(D-F) OCRs were continuously measured for 12h in freshly isolated GMPs from (D) young and (E) old mice that were grown in glucose-restricted medium (1 mM=R), (F) Quantification of the last time point (635 min), p-values were calculated by unpaired t-test comparing the depicted OCR to the OCR under normal glucose. The Data were normally distributed (Shapiro-Wilk test) after log transformation. Error bars represent SD.

(G-K) NanoString mRNA analysis of stress response genes in freshly-isolated HSCs from young (Y) and old (O) mice that were exposed to ad libitum (AL) or dietary restriction (DR diet for 2 weeks) of NAD-precursor treatment (NR) or a combination of NR plus DR (=NR/DR). The dotted lines represent the q-value of 0.05. Genes above this line are significantly regulated in the indicated comparisons. Note that single treatments of DR-alone or NR-alone lead to an induction of health-promoting stress response genes in young mice, but not in old mice. In old mice, only the combination of NR/DR leads to an induction of health-promoting stress response genes.

FIG. 14 (A) Young (2-3 months) and old (22-24 months) mice were exposed for 2 weeks to ad libitum diet (AL), dietary restriction (DR=CRD), NAD-precursor supplementation (=NR), NR/DR-combination of NR plus DR. Two weeks after initiation of the dietary treatments, 200 freshly isolated HSCs (CD150⁺CD34⁻LSK) of individual donor mice were transplanted together with 1×10⁶ competitor total bone marrow cells into lethally irradiated recipients. (A) quantification of the chimerism of donor-derived cells in white cells of the peripheral blood in recipient mice, 6 months after transplantation. N=5-6 recipients/donor per group. Data were normally distributed (Shapiro-Wilk test); p-values were calculated by 2-way ANOVA with Tukey's multiple comparisons test. Error bars depict SD. (B) Kaplan Meier survival analysis in cohorts of mice that were exposed to DR-alone, NR-alone, or NR/DR co-treatment compared to AL-feeding. Dietary interventions were started at the age of 22-24 months and continued over lifetime. The x-axis shows the time after start of the treatment. P-values were calculated using Mantel-Cox test. The p-values that are shown next to the intervention naming refer to the comparison of the intervention to the AL-diet group.

FIG. 15—Old (22-24 months old) mice were exposed for 2 weeks to the indicated diet: ad libitum (AL), Metformin (met), NAD-precursor (NR), NRM—combination of NR plus Metformin. Curves show respirometry analysis of the OCR of freshly isolated CMPs. The OCRs were continuously measured for 12h in freshly isolated CMPs grown in glucose-restricted medium (1 mM=R). Note that NR synergizes with Metformin (a mitochondrial stress-inducing CRM) to induce mitochondrial respiration—a critical parameter to induce stress signaling and health benefits.

### EXAMPLES

The invention is further described by the following examples. These are not intended to limit the scope of the invention, but represent preferred embodiments of aspects of the invention provided for greater illustration of the invention described herein.

Here we analyzed whether the time of intervention (young adult vs. late in life) would change the outcomes of DR on metabolic plasticity, stress responses, and functional improvements in hematopoietic stem and progenitor cells (HSPCs) of laboratory mice (C57Bl/6J). The focus on the hematopoietic system had the advantage that pure population of phenotypically defined stem cells (HSCs) and early progenitor cells (HPCs) could be employed for metabolic as well a functional tests such as the assessment of the repopulation capacity of HSCs in transplantation experiments in vivo. Moreover, this approach minimized the influence of aging-related changes in cell composition (that occur in many tissues) on the results. Our work reveals experimental evidence that aging-associated mitochondrial dysfunction abrogates the metabolic plasticity of HSPCs to respond to changes in nutrient availability. HSPCs from young mice adapt to DR-induced reduction in nutrient availability by activation of mitochondrial metabolism, metabolic stress responses, and mitophagy, resulting in improved stem cell function in transplantation assays in vivo. In contrast, HSPCs from old mice fail to enhance mitochondrial function or to activate stress response pathways in response to DR. Consequently, HSCs from old DR-exposed mice do not show an enhancement in functionality compared to HSCs from ad libitum (AL) fed mice. Of note, the supplementation of the NAD precursor, NR (Mouchiroud et al., 2013), rescues mitochondrial function, metabolic plasticity, nutrient sensing, and the regulation of stress response pathways in HSPCs from old mice, whereas chemical mitotoxins induce aging-like loss of metabolic plasticity in HSPCs from young mice. These results show that mitochondrial function itself represents an essential factor for DR-mediated induction of metabolic and functional enhancements of HSPCs. These responses to nutrient availability are disrupted by aging-associated mitochondrial dysfunction but reinstalled by NR supplementation.

Results of the Examples 1-8

### Example 1: Body Weight Stabilization and Improvements in HSC Function in Response to DR are Abrogated in Aged Versus Young Mice

To analyze whether the age at intervention would impact on the outcome of DR, young mice (3-5 months) and old mice (20-22 months) were switched to dietary restriction (DR: 30% reduction of the individual food consumed under ad libitum—AL—conditions) or kept under AL feeding (see materials and methods). All mice were kept in single cage housing (on AL diet starting 2 weeks before the diet assignment) to accurately monitor individual food intake. The experimental cohorts of mice were followed for 4 months using the same single mouse housing regiment. During the first 9 days after switching the diet to DR, mice from both age groups lost ˜10-20% of the body weight compared to the pre-intervention body weights (FIG. 1A,B). Thereafter, young mice completely recovered pre-intervention body weight, whereas old mice continued to lose body weight up to 25% at 53 days after switching to DR (FIG. 1A,B). Previous work from our group revealed that DR compared to AL diet has the potential to delay early aging of HSCs when the intervention is started in young, 3 months old mice and continued for 9 months. In this intervention period, DR strongly improved the repopulation capacity of HSCs (Tang et al., 2016). To determine the impact of intervention timing (old vs. young age) on the potential of DR to ameliorate the functional decline of HSCs during aging, young adult mice and old mice that were exposed to DR vs. a continuation of AL diet for 4 months followed by an analysis of the repopulation capacity of HSCs. Competitive transplantation of freshly isolated HSCs (Lin⁻Sca1⁺c-Kit⁺(=LSK)Flt3⁻CD34⁻CD150⁺) from the dietary intervention cohorts into lethally irradiated young recipients showed that 4-month DR vs. AL diet was sufficient to induce a significant improvement in HSC function from young mice to repopulate peripheral blood (FIG. 1C), total bone marrow (FIG. 2A), and HSC pools in bone marrow (FIG. 2B) of recipient mice. In contrast, the exposure of old mice to DR vs. AL diet did not lead to any significant improvement in the repopulation capacity of HSCs (FIG. 1C, FIG. 2A,B). Together, these results indicated that young mice exhibit a stabilization of body weights and improvements in HSC function in response to DR vs. AL diet; but these DR-mediated effects are abrogated in old mice.

### Example 2: Aging Abrogates Metabolic Plasticity of HSPCs to Respond to Changes in Nutrient Availability In Vivo

The above results showed that young mice may have the capacity to metabolically adapt to DR within 2 weeks, in order to stabilize body weight in response to reductions in nutrient availability, while old mice lose this capacity (FIG. 1A,B). We speculated that this capacity of metabolic plasticity to respond to changes in nutrient availability contributed to the enhancement in HSC function in DR-exposed young mice, which was abrogated in the old. Studies in Saccharomyces cerevisiae and D. melanogaster indicated that increases in mitochondrial activity and respiration are important factors that contribute to enhance lifespan in response to DR (Lin et al., 2002; Zid et al., 2009).

To directly analyze whether age-related changes in metabolic plasticity may influence the outcome of DR in mice, HSPCs were freshly isolated from young and old mice that were kept on AL diet or being switched to DR for 2 weeks. This time point was chosen since the body weight data indicated that young mice had adapted during this time window and managed to stabilize body weight despite the continuous exposure to DR (FIG. 1A,B). Interestingly, respirometry revealed increases in basal and maximal respiratory activity of freshly isolated myeloid progenitor cell populations including common myeloid progenitors (CMPs: Lin⁻c-Kit⁺Sca1⁻CD34⁺FcγR^{-/low}) and granulocyte macrophage progenitors (GMPs: Lin⁻c-Kit*Sca1⁻CD34⁺FcγR⁺) of young mice in response to DR (FIG. 1D and FIG. 4C). In contrast, the respiratory activity of CMPs and GMPs from old mice showed no adaptation at this timepoint after initiation of DR; the respiratory activity remained unchanged as compared to AL fed mice (FIG. 1E and FIG. 2D).

The analysis of HSCs (CD150⁺CD34⁻Flt3⁻LSK) from the same mice revealed a decrease in lactate levels and unchanged pyruvate levels in response to 2-weeks DR in young mice (FIG. 1F,G). Concomitantly, a reduction in lactate dehydrogenase (LDH) level was observed (FIG. 1H). This suppression in LDH likely contributes to a reduced conversion of pyruvate to lactate, thereby promoting higher usage of pyruvate for mitochondrial oxidative metabolism—a highly efficient pathway for ATP production. In line with this interpretation, we observed a significant increase in ATP levels in HSCs from DR-exposed, young mice compared to AL-fed control animals (FIG. 11). In contrast to these DR-induced metabolic changes of HSCs from young mice, HSCs from old mice exhibited pre-existing lower levels of lactate under AL-condition (FIG. 1F) and in response to DR exhibited an increase in lactate and LDH expression without any rise in ATP levels (FIG. 1F-I).

Together, the above experiments indicated that HSPCs from young mice adapt to changes in nutrient availability, resulting in increases in mitochondrial respiration and ATP production. However, HSPCs from old mice fail to adapt to changes in nutrient availability in a similar way.

### Example 3: Aging Associates with Mitochondrial Dysfunction in Murine HSPCs

Decreases in mitochondrial function occur in various tissues during aging and in aging associated diseases (Sun et al., 2016). To determine if aging-associated metabolic compromise could represent a possible cause for the loss of metabolic adaptation in old HSPCs, the mitochondrial metabolism was analyzed in HSPCs from young and old mice that were kept on AL diet. FACS analysis revealed a reduction in the mitochondrial membrane potential (MtMP) in HSCs of old mice compared to young mice (FIG. 3A). This coincided with elevated levels of reactive oxygen species (ROS) in HSCs (FIG. 3B). A reduction in the MtMP was also observed in the total population of multipotent progenitor cells (tMPP, CD34⁺LSK, containing myeloid primed and lymphoid primed MPP) but not in myeloid progenitors (MPs) or differentiated, Lineage-marker positive (Lin⁺) cells (FIG. 4A-C). The fraction of MPs (Lin⁻c-Kit*Sca1⁻) includes CMPs, GMPs, and megakaryocyte-erythroid progenitors (MEPs). Increases in ROS levels occurred in all subpopulations of the hematopoietic system that we tested, including total MPP population, MPs, Lin⁺ cells (FIG. 4D-F). To further analyze mitochondrial function, respirometry was performed on freshly isolated HSPCs from young and old mice. Due to the limited number of HSCs (especially in young mice) these experiments were carried out on MPPs and MPs. Respirometry of 5-6 mice per group indicated that MPPs and MPs from old mice compared to young mice had higher oxygen consumption rates (OCR) (FIG. 3C,D).

While DR-induced increases in OCR in combination with increases in ATP in HSPCs of young mice (FIG. 1D,I) indicated an activation of effective mitochondrial metabolism, the aging-associated increases in the OCR of HSPCs from AL fed mice in combination with the decrease in the MtMP and increases in ROS likely indicated an aberrant, non-effective metabolism of dysfunctional mitochondria in HSPCs from old mice. To substantiate this interpretation we analyzed ATP levels—an indicator of effective mitochondrial metabolism (Bonora et al., 2012) by mass spectrometry in freshly isolated HSPCs from young vs. old mice. This analysis showed a decrease in ATP levels in several subpopulations of HSPCs from old vs. young mice supporting the conclusion that mitochondrial metabolism was inefficient in old vs. young HSPCs (FIG. 5A).

Mitochondria also influence the cellular pools of NAD/NADP (Xiao et al., 2017). Mitochondria oxidative metabolism needs NADH as electron donor for oxidative phosphorylation, which in turn leads to production of NAD+, an essential co-substrate to several enzymes, including the Sirtuins, PARPs, and others (Xiao et al., 2017). In addition, mitochondrial ROS clearance consumes NADPH, which functions as an important redox scavenger (Xiao et al., 2017). The cellular levels of NADPH (FIG. 3B) and NADH (FIG. 3C) were significantly decreased in different subpopulations of HSPCs from old compared to young mice, while differences in NAD+(FIG. 5D) and NADP (data not shown) were not significant. In addition to the reduction in NADPH levels, enzymes that are crucial in redox homeostasis were significantly downregulated in MPPs during aging (FIG. 5E). Together, these results indicated that mitochondrial dysfunction in HSPCs of old vs. young mice results in reductions in ATP levels as well as in disturbances of NAD/NADP pools and redox homeostasis.

As alterations in NAD/NADP pools can also lead to disturbed expression of nuclear/mitochondrial encoded genes (Cantó et al., 2015), we analyzed the mRNA expression of several key factors for mitochondrial biogenesis and function (FIG. 5F-K), including nuclear (Nrf1, Tfam) and mitochondrial encoded genes (Nd1, Cytb, Co1, Atp6). All these genes were strongly downregulated in HSCs from aged compared to young mice, especially in the population of CD41⁺HSC⁻ a subpopulation of HSCs, which increases during aging and is characterized by a loss of function and myeloid skewed output (Yamamoto et al., 2018). One of the downregulated genes, Nd1 (NADH-ubiquinone oxidoreductase chain 1), is essential for the binding of NADH to complex 1, which is required for NADH to act as an electron donor at the start of the electron transport chain (Walker, 2009). These data suggested that diminished levels of NADH and its binding partner at the mitochondrial respiratory complex I could contribute to defects in mitochondrial function in HSPCs from old mice.

### Example 4: HSPCs from Old Mice Exhibit Enhanced, Cell-Intrinsic Dependency on Glycolysis and Abrogated Responses to Mitochondria Inhibition

The above data suggested that chronic mitochondrial dysfunction could contribute to the failure of metabolic plasticity that was observed in HSPCs from old mice in response to DR in vivo. To determine whether aged HSCs carry cell intrinsic alterations in mitochondrial metabolism, the proteome of highly purified HSCs (CD150⁺CD34⁻LSK) from our recent publication (Chen et al., 2019) was analyzed for changes in proteins that regulate glycolysis or oxidative mitochondrial metabolism. This analysis revealed a significant upregulation of glycolytic enzymes in HSCs from old compared to young mice (FIG. 6A) including hexokinase-1 and -2 (HK1 and HK2)—catalyzing the initiation of glycolysis (FIG. 6A). In contrast, the expression of enzymes involved in oxidative metabolism were reduced in HSCs from old compared to young mice including pyruvate dehydrogenase A and B (PDHA1 and PDHB) catalyzing oxidative decarboxylation of pyruvate—an essential step for the generation Acetyl-CoA, which is required to initiate oxidative glucose metabolism in mitochondria (FIG. 6B). Aging-associated up-regulation of key-activators of glycolysis (such as PKM) and concomitant reduction in enzymes involved in oxidative metabolism also occurred in the other 2 subpopulation of HSPCs, CMPs and GMPs (FIG. 7 A,B), which we also analyzed.

To test the functional relevance of these aging-associated changes in the proteome of HSCs, freshly isolated HSCs were exposed to treatment with oligomycin (an inhibitor of mitochondrial oxidative phosphorylation) or to treatment with 2-Deoxy-D-glucose (2-DG) in culture. 2-DG is an inhibitor of phosphoglucose isomerase and hexokinase, the first 2 enzymes initiating glycolysis (Ralser et al., 2008) In response to glycolysis inhibition, HSCs from old mice compared to HSCs from young mice were hypersensitive and exhibited a stronger reduction in cell numbers 5 days after initiation of the treatment (FIG. 6C). In contrast, in response to inhibition of mitochondrial oxidative metabolism, HSCs from old mice compared to HSCs from young mice were less responsive to reduce cell growth (FIG. 6C). Similar results were obtained after inhibition of nutrient influx into mitochondria. In this experiment, freshly isolated HSCs were incubated with compounds that inhibit the influx of nutrients into mitochondria (BPTES for glutamine, Etomoxir for fatty acids, or UK5099 for pyruvate). When exposed to such inhibitors (alone or in combination), HSCs from young mice exhibited a strong reduction in cell numbers 5 days after induction of the inhibitor treatment, whereas the sensitivity of HSCs from old mice to respond with a reduction in cell growth was significantly reduced (FIG. 6D,E). Of note, there was no significant difference in growth of cultured, control-treated HSCs from old vs. young mice indicating that growth-associated differences in nutrient demand were not responsible for this phenotype. Together, these data indicated that HSPCs from old compared to young mice show an increased dependency on glycolysis, but a reduced dependency on mitochondrial metabolism. These data provided functional evidence that HSPCs from old mice exhibit mitochondrial dysfunction, which by itself may reduce the plasticity of old HSPCs to respond to inhibition of mitochondrial metabolism

To experimentally test whether mitochondrial dysfunction would cause an abrogation of adaptive responses of HSCs to reduction in nutrient availability, young mice were exposed the mitochondrial uncoupler FCCP or vehicle (two injections: day-1 and day-4). One week after the first injection, the mice were analyzed and freshly isolated HSCs of FCCP-treated vs. DMSO-treated mice were exposed in culture to the 3 above described inhibitors of mitochondrial nutrient uptake—a combination of BPTES, Etomoxir, and UK5099, referred to from here on as “3I”. Interestingly, 1-week of FCCP treatment resulted in a dose dependent increase in the number of phenotypic HSCs in bone marrow (FIG. 6F)—a known feature of HSC aging. This finding also stood in line with a previous study showing that mitochondrial inhibition increases the self-renewal of cultured HSCs (Vannini et al., 2016). Inhibition of mitochondrial nutrient flow (3I-treatment) led to significant reduction in growth of HSCs from control treated mice, but HSCs from FCCP-treated mice exhibited a complete abrogation in responding to 3I-treatment by reduction in cell expansion (FIG. 6G). These data provided a proof of concept that mitochondrial function by itself abrogates the response of cultured, freshly isolated HSCs to reduce growth rates in response to reductions in mitochondrial nutrient flow.

### Example 5: Aging-Associated and Chemical-Induced Mitochondrial Dysfunction Abrogates Metabolic Plasticity in HSPCs

Our above data indicated that HSPCs from old mice exhibit mitochondrial dysfunction (FIGS. 3 and 5), and strongly depend on glycolysis but not on mitochondrial oxidative metabolism (FIGS. 6A-C), and—similar to FCCP-exposed HSCs from young mice—do not respond with growth inhibition to changes in nutrient availability (FIGS. 6D,E,G). Increases in mitochondrial metabolism have been shown to represent a key element of metabolic plasticity and functional enhancement induced by DR-mediated decreases in nutrient availability (reviewed in Guarente 2008). Our data revealed a failure of HSPCs from old compared to young mice to induce mitochondrial metabolism in response to DR (FIGS. 1D-I and FIGS. 2C,F) coinciding with a failure to stabilize body weight and to enhance HSC function as seen in young, DR-exposed mice (FIGS. 1A-C). Together, these findings suggested that aging-associated mitochondrial dysfunction abrogates metabolic plasticity, which itself is required to enhance stem cells and organismal functions in response to DR.

To further test this hypothesis, we analyzed the adaptability of mitochondrial metabolic pathways in freshly isolated HSPCs from young and old mice. First, we exposed freshly isolated HSCs to Oligomycin or to control treatment and we analyzed the RNA expression of Lactate dehydrogenase A (Ldha) and Pyruvate dehydrogenase A (Pdha) 3 hours after isolation and drug treatment. This experiment revealed a significant suppression of Ldha expression and a concomitant increase in Pdha expression in HSCs from young mice but HSCs from old mice were irresponsive (FIG. 8A,B). Second, we measured the activity of LDH and PDH in freshly isolated HSCs that were exposed for 20 hours to Oligomycin. HSCs from young mice responded to the treatment with a reduction in LDH activity, while PDH activity was maintained, thus responding with a shift of nutrient towards mitochondrial metabolism (FIG. 8C,D). Again HSCs from old mice were irresponsive (FIG. 8C,D). A similar mode of gene regulation was also observed for Nrf1 and Hif1α—two important metabolic stress response genes (FIG. 8E,F). Third, we analyzed OCR in freshly isolated CMPs that were treated for 12 hours with inhibitors of mitochondrial nutrient flow (3I) compared to control treatment. As outlined above, CMPs were used for these respirometry experiments, which need higher cell numbers and thus cannot be conducted on HSCs. Cultured CMPs from young mice showed a higher OCR compared to CMP from old mice (FIG. 8G). This increase in OCR in young compared to old CMPs was different from freshly isolated MPs (FIG. 3D) likely due to culture induced activation of OCR in young CMPs, which was impaired in old CMPs by pre-existing, chronic mitochondrial dysfunction. In response to 3I-treatment, CMPs from young mice reacted with a significant reduction in OCR, whereas CMPs from old mice remained unresponsive (FIG. 8G).

Together, these results support a model indicating that mitochondrial dysfunction itself stalled HSPCs from old mice in metabolically compromised state, which resulted in a loss of metabolic plasticity to respond to changes in mitochondrial function/nutrient flow into mitochondria. Of note, this response was different from DR-induced responses as the latter were inducing increases in OCR in HSPCs from young mice (FIG. 1D, FIG. 2C). It is conceivable that this difference is due to the experimental set up, where an overall reduction in nutrient availability by 30% in an in vivo organism leads to an increased efficiency in mitochondrial function and OCR, whereas the acute shut down of mitochondrial function in the culture system does not allow for the same kind of adaptive response. Despite these methodological differences, the immediate metabolic responses of young HSPCs after acute mitochondrial inhibition in culture and the abrogation of such a response in old HSPCs, support the concept that pre-existing mitochondria dysfunction (as seen in aging) by itself leads to abrogation of metabolic plasticity of HSPCs. To experimentally test this conclusion, CMPs from FCCP-treated vs. control treated young mice (see above) were exposed for 12 hours to 3I-treatment in culture and adaptive response in OCR were measured by Seahorse. In vivo pre-treatment with FCCP led to a dose dependent reduction in the OCR of freshly isolated CMPs indicating that the FCCP-treatment led to mitochondrial dysfunction. Of note, this coincided with a dose dependent loss in the responsiveness of CMPs from FCCP-treated mice to reduce OCRs in response to 3I-treatment in culture—thus mimicking the aging phenotype (FIG. 8H).

### Example 6: Aging Impairs Nutrient Sensing, Transcriptional Stress Responses, and the Induction of Clearance Pathways in HSPCs

Nutrient sensing pathways represent a prerequisite to activate stress response pathways that enhance cellular fitness in response to decreases in nutrient availability (Fontana et al., 2010). These responses are thought to increase metabolic fitness and to direct biomolecule and metabolic resources away from growth pathways toward an increased usage in repair and maintenance pathways (Geach, 2016). The mTOR signaling pathway controls major growth promoting signals and the pathway is suppressed in response to decreases in nutrient availability (Ochocki and Simon, 2013). FACS analysis revealed a suppression of phosphorylated (active) mTOR and phosphorylated ribosomal protein S6 (p-S6—a downstream target of mTOR) in HSCs of young mice that were exposed to 2-week DR treatment compared to AL-fed control mice (FIG. 9A,C). HSCs from AL-fed, old mice exhibited lower levels of p-mTOR and p-S6 compared to young mice but in response to DR-treatment p-mTOR and p-S6 levels were not significantly downregulated (FIG. 9B,D). A similar response pattern was also observed for p-AKT (FIG. 10A,B). NanoString analysis of the expression of a focused set of 52 mRNAs of metabolic stress response genes revealed an upregulation of 16 genes in HSCs of 2-week DR-exposed young mice compared to AL-fed control mice (FIG. 9E and FIG. 10C). These genes were involved in a broad variety of biological processes, such as autophagy and mitophagy induction (Parkin, Sirt1), cell cycle control (p57), mitochondrial activity and biogenesis (Polg, Ucp2, Idh1, Cox4i1), chromatin remodeling and mtUPR (Sirt7), insulin/lgf signaling (Hmga2, Irs2), and NAD biosynthesis (Naprt, which catalyzes the first step in the biosynthesis of NAD from nicotinic acid). Of note, HSCs from old mice showed a complete loss of responsiveness and none of the 52 tested stress response genes were regulated in response to 2-week DR-treatment compared to AL-fed, old mice. The 16 genes that responded to DR in young mice only showed slight, non-significant regulation in response to 2 weeks DR-treatment compared to AL-fed, old mice (FIG. 9F and FIG. 10D).

The above data indicated that HSPCs from old mice compared to HSPCs from young mice exhibit defects in nutrient sensing and in the activation of stress response genes after short-term exposure (2 weeks) to DR. Nutrient sensing and induction of metabolic stress responses have been shown to lead to the activation of cellular and subcellular clearance mechanisms in response to DR that contribute to the beneficial effects of DR on disease prevention and lifespan extension. Examples include the induction of apoptosis (Dunn et al., 1997; Holt et al., 1998), autophagy/mitophagy (Hansen et al., 2008; Jia and Levine, 2007; Morselli et al., 2010), and unfolded protein responses (Mohrin et al., 2015). Interestingly, defects in clearance systems have been implicated to contribute to aging of HSCs (Ho et al., 2017; Mohrin et al., 2015). To test whether impairments in the induction of cellular or subcellular clearance mechanisms may contribute to the failure of HSPCs from old mice to adapt to DR to increase metabolic and cellular fitness, we followed the induction of autophagy/mitophagy and changes in HSPCs numbers in mice that were exposed to DR or AL control diet for 1-2 weeks.

In young mice, 2-week DR exposure induced a significant decline in HSC and MPP numbers compared to AL fed animals; however, this response was abrogated in aged mice (FIGS. 9G,H and FIG. 10E,F). These data suggested that the clearance of metabolically compromised HSCs contributes to increased metabolic fitness in response to DR and old mice appeared to be compromised in mounting such responses to DR. The aforementioned analysis on stress responses revealed an induction in the expression of subcellular clearance pathways in MPPs in response to DR in young mice, but not in old mice, including Parkin—an important regulator of inducible mitophagy, Sirt1—a regulator of general autophagy, Sirt7—a regulator of chromatin remodeling and mtUPR (see FIG. 9E,F). At the protein level, the expression of PINK1 (a marker and regulator of clearance of damaged mitochondria by mitophagy) was upregulated in cultures of freshly isolated, young MPPs in response to the inhibition of nutrient influx (3I-treatment) into mitochondria (FIG. 91). MPPs from old mice failed to mediate such a response (FIG. 91). Similar results were observed after exposure of freshly isolated MPPs to Oligomycin treatment (FIG. 10G). In addition, immunofluorescence co-staining of marker proteins of autophagy (LC3) and of mitochondria (TOM20) revealed that MPPs from young DR mice upregulated LC3 as compared to young AL-fed mice, whereas old mice exhibited elevated levels of LC3 expression during AL but failed to further increase LC3 upon DR (FIG. 9J). Immunofluorescence analysis revealed that the number of MPPs that exhibited co-localization of LC3 and TOM20 was reduced in DR-exposed compared to AL-fed young mice but not in DR-exposed compared to AL-fed, old mice (FIG. 10H,1). A likely explanation for these findings was that DR induced autophagy in MPPs from young mice, which led to clearance of damaged mitochondrial as indicated by a decreased LC3/TOM20 co-staining. In contrast, MPPs from old mice did not induce such DR-induced responses in mitochondrial clearance. Together, these data indicated that HSPCs from old mice exhibit a chronically increased level of dysfunctional autophagy, which could not be induced by DR.

### Example 7: Short-Term DR has the Capacity to Ameliorate 80% of the Differences in the Proteome of HSPCs from Young and Old Mice, but Age-Related Alterations of NAD Dependent Biological Processes Remain

To determine aging-related changes in the proteome of HSPCs and to test whether DR would have the capacity to revert such changes, a proteome analysis was conducted on freshly isolated GMPs and CMPs from young and old mice that were on continuous AL diet or exposed to DR for 2 weeks. These cell types were chosen because they show similar defects in responding to changes in nutrient availability as HSC (see above results) and are available in high enough number per mouse (100,000) to conduct a deep proteomics analysis. In our analysis we detected >4,900 proteins per sample across the different groups. The below description focuses on GMPs because this population is purer and shows less aging-related changes in cell composition compared to CMPs. However, the data on CMPs are similar to those presented here on GMPs. The mass spectrometry proteomics data have been deposited to the ProteomeXchange Consortium via the PRIDE (Vizcaino et al., 2015) partner repository with the dataset identifier PXD015557 at: http://www.ebi.ac.uk/pride.

The analysis revealed that a high percentage of DR-induced changes in the proteome of GMPs (>80%) occurred in an age-dependent manner, e.g. only in young or old mice, but not overlapping in both age-groups (FIG. 11A, B). Intriguingly, DR-induced changes in the entire proteome of GMPs from young mice showed a strong positive association with proteome changes in GMPs that occurred during aging of AL-fed mice (FIG. 12A). This finding indicates that old GMPs exhibit DR-like changes under AL conditions. This results stand in agreement with our data on DR-like changes in old AL mice in lactate metabolism (FIG. 1F,H, and FIG. 8A), nutrient sensing (FIGS. 9A-D) and autophagy (FIG. 9J). However, HSPCs from young mice activate adaptive response towards these DR-induced, aging-like changes, whereas HSPCs from old mice fail to do so (FIG. 1D,E,I; FIG. 2A,C,D; FIG. 9A-J). These results suggest, that mitochondrial dysfunction stalls HSPCs from old mice in a chronically compromised metabolic state, which is similar to DR but abrogates the responsiveness to changes in nutrient availability. Interestingly, DR-induced changes in the proteome of GMPs from old mice showed a strong negative association with proteome changes in GMPs that occurred during aging of AL-fed mice (FIG. 12A). Together, these age-dependent effects of DR led to a reduction in aging-associated protein expression changes in DR-exposed mice compared to AL fed mice as indicated by reduced spreading of the density plot of differentially expressed proteins in old vs. young mice that were exposed for 2 weeks to DR (red) or AL-diet (blue) (FIG. 12B). Analyzing the total number of significantly (q-value <0.05) expressed proteins in both cohorts revealed 896 differentially expressed proteins in DR-exposed mice compared to 1855 differentially expressed proteins in AL fed mice (p<2.22e-16, FIG. 12B). Despite this strong rescue in proteome expression changes, DR failed to induce metabolic plasticity and increases in mitochondrial metabolism in HSPCs of old mice compared to young mice (see above results).

To determine possible mechanisms that might be related to the failure of DR to re-induce metabolic plasticity in old mice, we analyzed proteins that remained differentially regulated in GMPs of old vs. young mice that were exposed for 2 weeks to DR (log 2 fold change in absolute number >0.25 as indicated by the dotted lines in the density plot of differentially expressed proteins in FIG. 12B). These proteins contained a small fraction of proteins that were already dysregulated in AL-fed old vs. young mice and not rescued by DR (n=360 proteins). Together, these data showed that DR reversed 80% of protein expression changes in GMPs that were present in old vs. young AL-fed mice and that only a small fraction remained to be differentially expressed in old vs. young DR-exposed mice (360/1855=19.4%). However, there was also a set of differentially expressed proteins in old versus young DR-exposed mice were not the proteins differentially expressed in old versus young AL-fed mice (n=536 proteins) indicating that age-related differences in the response to DR contributed to protein expression differences in old vs. young DR-exposed mice.

To determine which biological processes were differentially regulated in the proteome of old vs young DR-exposed mice, “ingenuity pathway analysis—IPA” was conducted on all proteins that showed a significant difference (q-value <0.05) in expression in GMPs of DR-exposed, old vs. young mice. This analysis revealed the strongest enrichment for two biological processes (FIG. 7C)—(i) a decline of sirtuin-mediated signaling pathways (p=1×10⁻¹³) and (ii) a deregulation of proteins that are indicative of mitochondrial dysfunction (p=1×10⁵). Interestingly, both of these biological processes are strongly dependent on availability of the coenzyme NAD (Guarente, 2008). Of note, NADH-ubiquinone oxidoreductase (NDUF) subunits of the mitochondrial respiratory complex I represented a prominent cluster subsumed under the term “Sirtuin pathway” in the IPA analysis (FIG. 12C, Table-1). Interestingly, the vast majority of differentially expressed NDUFs showed a significant upregulation in DR-exposed old vs. young mice (FIG. 12D) possibly reflecting a compensatory reaction to reductions in NADH pools, which was seen in some subpopulations of HSPCs in old mice (FIG. 5C).

### Example 8: NR Supplementation Re-Installs Mitochondrial Function and Metabolic Plasticity in HSCs from Old Mice to Respond to Changes in Nutrient Availability

To test the hypothesis that NAD(P) availability limits the potential of HSPCs from old mice to respond to changes in nutrient availability, freshly isolated HSCs from young and old mice were exposed to inhibitors of mitochondrial nutrient uptake. Freshly isolated HSCs from young and old mice were pre-treated for 12 hours with NR—a potent progenitor of NAD synthesis, which has been shown to promote mitochondrial activity and lifespan extension in mice (Zhang et al., 2016)—or with a vehicle control. In line with our previous results (FIGS. 6D,E and 8G), inhibition of mitochondrial nutrient availability by 3I-treatment of HSCs from young mice suppressed cell proliferation (FIG. 12E) and reduced oxygen consumption (FIG. 12F) in freshly isolated HSCs from young mice. These adaptive responses were severely compromised in control (non-NR-treated) HSCs from old mice (FIG. 12E,F). Interestingly, NR treatment of HSCs from old mice rescued oxidative metabolism to a similar level as observed in HSCs from young mice (FIG. 12F). This rescue in mitochondrial function of old HSC coincided with a rescue of metabolic plasticity to respond to an inhibition of nutrient flux into mitochondria (3I-treatment) by suppression of cell proliferation (FIG. 12E) and by reduction of oxidative metabolism (FIG. 12F). The transcription of stress response genes was strongly altered in nutrient deprived HSCs from old vs. young mice (FIG. 12G, Table-2) and NR-treatment had no significant effects on these pre-existing, age-related changes in HSCs that were not exposed to inhibition of mitochondrial nutrient flow (no 3I-treatment) (FIG. 11C-E). In contrast, NR-treatment reverted changes in the transcription of stress response genes in old vs. young HSCs that were exposed to an inhibition of mitochondrial nutrient flow (3I-treatment) (FIG. 12H, Table-3). These data indicated that NR-treatment has the potential to rescue the responsiveness of old HSCs to react to changes mitochondrial nutrient availability.

Discussion of the Results of Examples 1-8

The present study provides direct experimental evidence that aging associated mitochondrial dysfunction abrogates metabolic plasticity of HSPCs to respond to reductions in nutrient availability. This leads to a failure of DR to induce metabolic and functional enhancements of HSCs in old compared to young mice. The study also demonstrates that the supplement of NAD precursors reinstalls mitochondrial function and the responsiveness of old HSCs to react to reductions in nutrient availability. Taken together these results determine mitochondrial function as an essential factor for metabolic and functional responsiveness of HSPCs to changes in nutrient availability. As aging related loss in mitochondrial abrogates metabolic plasticity, the study implies that the supplementation of NAD precursors would be required to reinstall DR-mediated improvements in HSC function and organism aging.

Mitochondrial Dysfunction Limits Metabolic Plasticity to Adapt to Changes in Nutrient Availability in Aging HSPCs.

Increases in mitochondrial nutrient flow and oxidative metabolism have previously been implicated to contribute to the extension of lifespan in response to DR in different model organisms including yeast (Lin et al., 2002), C. elegans (Bishop and Guarente, 2007; Weir et al., 2017), and mice (Nisoli et al., 2005). Studies on yeast have shown that cytochrome-c-dependent mitochondrial respiration is required for DR-mediated lifespan extension and that over-activation of mitochondrial biogenesis by itself leads to lifespan extension, which could not be enhanced any further by DR (Lin et al., 2002). These results suggested that increases in mitochondrial respiration represent an integral hub required for the lifespan extension in response to DR. This assumption was supported by studies on C. elegans showing that the induction of skn1-dependent signals in neurons is required for mitochondrial activation in somatic tissues and for DR-mediated lifespanextension (Bishop and Guarente, 2007). Importantly, aging-related limitations in mitochondria activation and its mechanistic consequences on cellular and organismal fitness in response to changes in nutrient availability have not been delineated.

The current study shows that the aging-associated failure to increase mitochondrial respiration represents a major cause for the loss of metabolic plasticity of aged HSPCs to adapt to changes in nutrient availability. The study reveals that the in vivo application of mitotoxins (FCCP) to young mice leads to mitochondrial dysfunction, which is sufficient to abrogate the metabolic responsiveness of HSPCs to adapt to changes in nutrient availability thus mimicking the aging phenotype. Furthermore, the supplementation of NAD precursors reinstalls mitochondrial activity with the subsequent responsiveness of aged HSCs to adapt (modify) proliferation rates, oxidative metabolism, and the expression of stress signaling pathways in response to changes in nutrient availability. These findings indicate that NAD dependent mitochondria dysfunction is a causative factor for the loss of metabolic and functional plasticity of HSPCs during aging. These results could have important implications for our understanding of stem cell and organism aging in general. Previous studies showed that transient inhibition of mitochondrial function in C. elegans only resulted in lifespan extension when applied during developmental stages but not when given to adult worms (Lin et al., 2002). It is conceivable that the decline in mitochondria function during the maturation and aging of C. elegans limits metabolic plasticity and the induction of lifespan extension. In agreement with this interpretation, DR-induced effects on reduction of mortality rates in mice are greatly impaired during aging, coinciding with a reduced capacity of white adipose fat tissue (WAT) to increase phospholipid-dependent mitochondrial biogenesis (Hahn et al., 2019)

Supplementation of NAD Precursors Reinstalls Metabolic Plasticity and the Responsiveness of Old HSCs to Changes in Nutrient Availability.

It has been proposed that the activation of mitochondrial metabolism leads to intra-mitochondrial and intracellular increases in NAD/NADP ratios, which in turn contribute to the activation of NAD-dependent stress responses that increase cellular fitness (Guarente, 2008). The current study shows that DR has the capacity to revert large proportions of aging-related alterations in the proteome of HSPCs. However, proteins involved in NAD dependent biological processes are not normalized but strongly dysregulated in old vs. young DR-exposed mice. These processes include a reduction in Sirtuin signaling and an increase in the expression of protein of complex I of the mitochondrial respiratory chain, which are required for initiation of oxidative phosphorylation (FIG. 12). The upregulation of mitochondrial complex I proteins in old HSPC (FIG. 12D) in combination with the failure to induce mitochondrial respiration in response to DR (FIG. 2D) likely reflects an attempt to activate mitochondrial respiration, which, however, is not successful due to low levels of NADH, which is needed as an electron donor for electron transport chain. As a consequence, mitochondria respiration is not induced in response to DR despite the upregulation of complex I proteins. In turn, the generation of NAD+ and the activation of Sirtuin signaling (which requires NAD+ as a cofactor) are not boosted in response to DR.

Previous studies revealed that impairments in Sirtuin signaling (Brown et al., 2013; Mohrin et al., 2015) and autophagy (Ho et al., 2017) contribute to the aging-associated decline in HSC function. The current study indicates that aging-associated mitochondrial dysfunction abrogates nutrient sensing and the regulation of stress response pathways in old HPSCs. However, these deficiencies can be reversed by NR supplementation, which improves mitochondrial function and the regulation of stress signaling pathways in activated HSCs from old mice in culture. Studies on the in vivo application of NR to young mice also revealed evidence that NR reduces mitochondrial activity in HSCs by enhancing autophagy mediated clearance of mitochondria (Vannini et al., 2019). It is possible that NR dependent improvements in autophagy-mediated clearance of dysfunctional mitochondria also contribute to the here described potential of NR to rescue mitochondrial oxidative metabolism and consequently the responsiveness of old HSPCs to adapt to changes in nutrient availability.

Together, the current study provides experimental support that in old mice chronic mitochondrial dysfunction stalls HSPCs in a compromised metabolic state, leading to a loss of nutrient sensing and metabolic plasticity to respond to changes in nutrient availability. These defects limit the capacity of DR to induce mitochondrial metabolism, adaptive stress responses, and functional enhancement of HSPCs in old mice. It is conceivable that the aging-related failure in nutrient sensing and in induction of stress signaling pathways may also contribute to the functional decline of HSCs during aging, by for example abrogating circadian responses to changes in nutrient availability induced by pauses in nutrient uptake. The study identifies deficiencies in NADH as a key factor, which limits the induction of metabolic plasticity and adaptive responses in old HSCs. Importantly, the supplementation of NAD precursors reinstalls mitochondrial function, metabolic plasticity and adaptive responses of old HSCs to respond to reductions in mitochondrial nutrient flow. These findings imply that combining dietary interventions with the supplementation of NAD precursors have the potential to improve stem cell and organism function at old age.

### Example 9: NAD Precursor Supplementation in Combination with CRD Rescues Mitochondria Function, the Induction of Stress Response in Hematopoietic Stem and Progenitor Cells of Old Mice—Results and Discussion

Based on our results, we reasoned that mitochondria enhancement could possibly re-install mitochondria plasticity to respond to changes in nutrient availability and, thereby, the capacity of DR (=CRD) to induce transcriptional responses and functional improvements of HSCs of old mice. However, the potential of mitochondria enhancement to reinstall mitochondrial responses to nutrient deprivation has never been explored.

To experimentally test whether mitochondrial enhancement could re-empower DR (=CRD) in old mice, young and old mice were exposed to NAD+ supplementation by feeding of nicotinamide riboside (NR). In addition, the mice were exposed for 2 weeks to DR (=CRD), starting 1 week after NR pre-feeding, or continuous AL feeding. Respirometry analysis of freshly isolated CMPs from young mice showed that either one of the single treatments (NR-alone or DR-alone) led to significant increases in mitochondrial respiration compared to HSCs from AL-fed, young mice, but there was no additive effect of NR/DR combination (FIG. 13A,C). In HSCs from old mice, neither of the single treatments was sufficient to induce mitochondrial respiration; it was only the NR/DR combination treatment that had the capacity to induce mitochondrial respiration compared to AL-fed mice (FIG. 13B,C).

Similar results were obtained when freshly isolated GMPs from the mice of the 2-weeks dietary intervention cohorts were exposed to glucose deprivation in culture—it was only the combination of NR/DR treatment that reinstalled the cell-intrinsic capacity of GMPs from old to activate mitochondrial respiration in response to glucose depletion (FIG. 13D-F).

To analyze whether the NR/DR-mediated re-installment of mitochondrial metabolic plasticity in HSCs from old mice could rescue the induction of metabolic stress signals, freshly isolated HSCs from 2-week dietary cohorts were analyzed with the same NanoString assay as described above. In HSCs from young mice, supplementation of NR-alone induced metabolic stress signals (FIG. 13G) and the transcriptional response was highly similar to the transcriptional response induced by DR-alone (FIG. 13H). In contrast, in HSCs of old mice neither of the single treatments, NR-alone (FIG. 13I) or DR-alone (FIG. 13J) was able to induce metabolic stress signaling. Strikingly, however, NR/DR co-treatment fully rescued the induction of metabolic stress signaling in HSCs of old mice (FIG. 13K), which was very similar to the transcriptional response seen in HSCs of young DR-exposed mice (FIG. 13H) or NR-supplemented mice (FIG. 13G).

Together, these results demonstrated that the capacity of dietary interventions to induce metabolic stress signaling depends strictly on the induction of mitochondrial activity, which in young mice can be achieved by either NR- or DR-treatment alone, whereas in old mice this can only be achieved by combining NAD precursor supplementation with DR-exposure.

### Example 10: NAD Precursor Supplementation in Combination with CRD Improves Stem Cell Function and Lifespan of Old Mice—Results and Discussion

Transplantation of freshly isolated HSCs showed that all 3 dietary short-term interventions (2 weeks of DR-alone (=CRD), NR-alone (=NAD precursor supplementation) or NR/DR co-treatment) increased the repopulation capacity of HSCs from young mice (FIG. 14A). In contrast, an increase in the repopulation capacity of HSCs from old mice was only seen in HSCs derived from NR/DR-co-treated donors but not in HSCs derived from old mice that were treated by either one of the single treatments (DR-alone or NR-alone) (FIG. 14A).

To test effects of these dietary intervention on organism lifespan, 4 cohorts of 22-24 months old female mice (n=14-15 mice per group) were exposed to long-term treatments (until the end of life or the occurrence of humane endpoints). Single treatment with DR or NR led to slight increases in lifespan compared to AL-fed control mice (FIG. 14B). However, these differences did not reach significance compared to AL-fed mice. In sharp contrast to the non-significant lifespan effects of single treatments, strikingly the NR/DR cotreatment of old mice led to a very strong, highly significant increase in life expectancy compared to AL-fed controls (p<0.0001, FIG. 14B) as well as in comparison to the treatment with NR-alone (p=0.0005, FIG. 14B) or DR-alone (p=0.0004, FIG. 14B). The NR/DR cotreatment cohort is currently still under investigation with 86% of the mice still being alive compared to only 15-30% of survival rates in the other 3 dietary cohorts.

Together, the data provide experimental evidence that aging-associated mitochondrial dysfunction abrogates the metabolic plasticity of HSPCs to respond to changes in nutrient availability by activating mitochondria respiration. These defects in turn impair DR-mediated induction of metabolic stress signals and health benefits of DR in old mice, such as HSC enhancement and lifespan elongation. The current work provides a mechanistic understanding for the failure of DR at old age and identifies a dietary intervention that reinstalls it. The study shows that mitochondria activation is a key mechanism, which fails to respond to DR in old vs. young mice. This failure can fully be rescued by NAD-precursor supplementation, which in turn enables DR (=CRD) to induce mitochondria activity, metabolic stress signaling, functional enhancement of HSPCs, and lifespan extension in old mice.

Importantly, the here described effect of late-life NR/DR treatment on overall lifespan appears to be very strong (>23% increase in median life expectancy compared to AL with currently 55% of the mice still being alive, p<0.0001) compared to the best, currently known dietary interventions that increase lifespan when applied to old mice, such as Spermidin or single NR treatment (6.9% in previous studies, 10% in this study with the effect not being significant) or single DR-treatment (no effect in previous studies, 10% in this study with the effect not being significant).

Given the global increases in size of the elderly population and in aging-associated diseases, a powerful, non-toxic, dietary intervention that increase health at old age would fulfill a huge unmet need. In humans, NAD precursor supplementation in combination with DR could represent a promising approach to achieve this goal.

### Example 11: NAD-Precursor Supplementation Cooperates with Mitochondria-Inhibiting CRMs to Induce Mitochondria Activity—Results and Discussion

To test whether NAD precursor supplementation would cooperate with specific CRMs that mimic a certain sub-pathway induced by CRD, different classes of CRMs were employed. We tested the combined effects of NAD supplementation with
(a) Metformin—a CRM, which induces mitochondrial stress
(b) Pterostilbene—a CRM which induces Sirtuin-dependent stress responses including autophagy
(c) Spermidine—a CRM, which induces autophagy (ad ownstream target of sirtuins (see above)

Old mice (22-24 months) were fed with CRMs (a-c) with or without combination with NAD-precursor supplementation for 2 weeks before analysis. Freshly isolated hematopoietic cells (CMPs) were tested for a rescue in responsiveness to activate mitochondria in response to glucose depletion—a marker of metabolic improvement induced by CRD in young mice or by a combination of NAD precursor supplementation plus CRD in old mice.

Of note NAD precursor supplementation showed synergistic effects on inducing mitochondria activity only when combine with metformin, which mimics the mitochondria stress induction of CRD. In contrast, NAD precursor supplementation showed no synergistic effects on inducing mitochondria activity when combined with pterostilbene mimicking the sirtuin/autophagy induction of CRD. Based on these results we anticipate that NAD precursor supplementation will also not show synergism with spermidine mimicking the induction of autophagy downstream of sirtuin activation in response to CRD.

Together these results show that NAD precursor supplementation has a great synergistic potential to induce mitochondria activity and health benefits in combination with the subclass of CRMs mimicking mitochondrial stress induced by CRD.

Methods Employed in the Examples

Mice:

All wild type mice used were C57BL/6 mice and obtained from Janvier. Mice were maintained in a specific pathogen-free animal facility in FLI with 12 hours of the light-dark cycle and fed with a standard mouse chow food. Experiments were conducted according to protocols approved by the state government of Thuringia (FLI17-006, FLI19-009, and O_KLR_18-20).

Dietary Restriction and Weight Curve

Prior to the start of dietary restriction (DR) experiment the daily food consumption of Young (2-3 months) and old (20-24 months) female WT C57BL/6 mice were measured for 2 weeks. Each mouse was placed in single cage. Mice that were exposed to DR were given 30% reduction of their daily food consumption. Mice in the control group (AL) were given unlimited access to ad libitum food (VRF1 from Sniff). The mice were always fed during the morning time. The weight of each mouse was measured before and during the experiment. The weight change for each mouse was calculated as follows: the weight of individual mouse at the indicated time point was divided by its initial weight one day before the start of DR experiment.

Dietary/Calorie Restriction Mimetics (CRM)

Old mice (22-24 month) were treated with CRMs:
(a) Metformin-Hydrochlorid (0,1% weight proportion of chow)—metformin is a CRM that mimics mitochondrial stress induced by CRD, metformin does so by inhibiting complex-1 of the electron transport chain (ETC) of the mitochondria.
(b) Perostilbene (120 mg/kg chow)—is a CRM, which leads to sirtuin activation. Sirtuin is also activated in response to CRD. Sirtuin activation induces stress responses including autophagy, which increases the quality of mitochondria by digesting dysfunctional mitochondria.
(c) Spermidine (3 mM in the drinking water)—is a CRM, which induces autophagy, Autophagy is induced by Sirtuin activation. It is thus a downstream target of Sirtuins (see above).

(a-c) CRMs were given to mice for 2 weeks, (c) CRM was given to mice for 2 months to reach systemic increases in spermidine. Mice were treated with the CRM-alone or in combination with NAD-precursor feeding (NR) for 2 weeks prior to analysis.

Transplantation

Two hundred freshly isolated HSCs (CD150⁺CD34⁻Flt3⁻LSK) from each individual mouse were transplanted via intravenous injection into lethally gamma-irradiated (12 Gy) young recipients (2-3 months) recipients together with 1×10⁶ of competitor bone marrow cells. During the first week of transplantation the mice were provided with antibiotic water (0.01%, Baytril). The repopulation capacity in peripheral blood was measured by BD LSR Fortessa every 4 weeks for the whole period of transplantation. Four months after transplantation the mice euthanized and bone marrow was taken for chimerism analysis by BD LSR Fortessa or BD FACS ARIA Ill.

FCCP Injection

Young C57BL/6 wild type mice (3 months) male mice were weighed before the injection. The mice were then injected intraperitoneally with low dose (1.2 mg/Kg), high dose (4.82 mg/Kg) of FCCP, or 10% DMSO as control (all drugs were dissolved in 10% DMSO. The drug concentration was adjusted so that the final volume of injection of each injection is 150 pL/mouse. The mice were injected at day 1 and day 4 after weighing. Then they were analyzed at day 6.

FACS Sorting and Analysis

Bone marrow (BM) cells were obtained and isolated from fore limbs, hind limbs, pelvis and spine. After completely cleaning off the muscle and connective tissue, all the bones were crushed in sterile PBS containing 2% heat-inactivated FBS (staining media) using mortar and pestle. For isolating specific populations in BM cells, single-cell suspensions were enriched for c-Kit⁺cells by sequentially staining with c-Kit-APC (Biolegend) antibody and anti-APC microbeads (Miltenyi Biotec) on ice. The c-Kit⁺cells separated and harvested by MACS Separation LS Columns (Miltenyi Biotec) on magnetic stand (Miltenyi Biotec). The enriched cells were then stained with linage antibody cocktail containing biotinylated antibodies against CD4 (Biolegend, 100508), CD8 (Biolegend, 100704), TER-119 (Biolegend, 116204), CD11b (Biolegend, 101204), Gr-1 (Biolegend, 108404) and B220 (Biolegend, 103222) at 4° C. for 30 min. After washing, cells were incubated with a mix of antibodies against FcYR-FITC, Flt3-PE, Sca1-PE/Cy7, c-Kit-APC, Streptavidin-APC/Cy7, CD34-Alexa Fluor 700, and CD150-Brilliant Violet 605. After staining, the cells were washed and resuspended with 1 mL staining media containing 0.5 uL DAPI (1 mg/mL) to exclude dead cells. The desired cell populations according to the cell surface markers were subsequently sorted by BD Aria III flow cytometer.

For FACS analysis, freshly isolated total BM cells (1×10⁷) were stained with antibodies to detect the desired surface markers. After antibody staining, BM cells were incubated with Rhodamin123 (Thermo)—PBS (1.25 μg/mL) to detect mitochondrial membrane potential, or incubated with CellROX reagent (Thermo, 1:500 diluted) in 200 uL PBS at 37° C. for 30 mins to detect ROS. After staining, samples were washed with 1 mL staining media for twice, resuspended in 500 uL staining media with DAPI (1 μg/mL) and then were kept on ice for FACS analysis. For intracellular antigen detection: p-mTor, (pS2448), p-S⁶ (pS235/pS236), p-Akt (S473), and Pink1, BM cells were fixed with 100 ul Fixation/Permeabilization Solution (Fisher scientific, BD 554714) for 10 min and stained with p-mTor (BD Biosciences, 563489), p-S6 (BD Biosciences, 560434), p-Akt (R&D, IC7794G), or Pink1 (Abcam, ab186259) antibody overnight. After staining, samples were washed with 1 mL perm wash buffer (Fisher scientific, BD 554714) twice and FACS analysis was performed using BD LSR Fortessa.

Analysis of Transplanted Mice

For analysis of chimerism in peripheral blood (PB) of transplanted mice, 30 uL of EDTA-collected blood was stained with CD45.2-Percp-Cy5.5 (Biolegend, 109828) CD45.1-PE (Biolegend, 110708) conjugated anti-mouse antibodies for 30 min on ice. The cells were then lysed with 1×RBC lysis Buffer (BD Biosciences, 555899) for 5 min. The cells were then washed with 1×PBS and centrifuged for 5 min at 300 g on 4° C. The cells were then resuspended in 2% FBS in PBS and analyzed by BD LSR fortessa. For the analysis of hematopoietic stem and progenitor cells (HSPCs) from transplanted mice 10×10⁶ total bone marrow cells were incubated with a lineage cocktail containing biotinylated antibodies against CD4 (Biolegend, 100508), CD8 (Biolegend, 100704), TER-119 (Biolegend, 116204), CD11b (Biolegend, 101204), Gr-1 (Biolegend, 108404) and B220 (Biolegend, 103222) at 4° C. for 30 min. After washing, cells were incubated with a mix of antibodies against CD45.2-Percp-Cy5.5 (Biolegend, 109828) CD45.1-PE (Biolegend, 110708), CD150-Bv605 (Biolegend, 115927), CD34-AF700 (eBioscience, 56-0341-82), Flt3-PE (Biolegend, 135306), FcYR-FITC (Biolegend, 101306), Sca-1-PE-Cy7 (Biolegend, 108114), c-Kit-APC (Biolegend, 105812), and streptavidin-APC-Cy7 on ice. (Biolegend, 405208)

Cell Culture

HSCs and progenitors were cultured in SFEM medium (STEM CELL Technologies, 09650) supplemented with 50 ng/mL mSCF (Peprotech, 250-03), 50 ng/mL mTPO (Peprotech, 315-14), 100 units/mL penicillin, 100 μg/mL streptomycin (Gibco, 15140-122). For testing the response of HSPCs to the blocking of glycolysis or mitochondrial ATP synthesis, 2-DG (Sigma, D8375) or oligomycin A (Sigma, 75351) were used. For the inhibition of nutrient uptake into mitochondria, three inhibitors (3I) were used. BPTES (Sigma, SML0601) blocks glutamate uptake, Etomoxir (Sigma E1905) blocks Fatty acids uptake, and UK5099 (Sigma PZ0160) blocks pyruvate uptake. The final concentration of each inhibitor in the culture medium is 6 μM. Same amount of DMSO was used in the medium as control. For determination of cell number after NR treatment 500 HSCs from young (2-3 months) and old (22-24 months) were pre-incubated either with 1 mM NR (provided by Prof. Johan Auwerx Lab) or DMSO as control for 2 days followed by 2 days treatment with the inhibitors (BPTES, Etomoxir and UK5099, 6 μM each) or same amount of DMSO as control. The cells were then analyzed by BD LSR fortessa.

Seahorse Analysis

Measurement of metabolic activity was done using XF96e according to the guidelines of Agilent Seahorse XF Cell Mito Stress Test (Agilent technologies). In brief, XF base medium (Agilent technologies, 102353) was supplemented with final concentration of 1 mM pyruvate, 2 mM glutamine, 10 mM glucose, 50 ng/mL mTPO, and 50 ng/mL mSFC. The medium was adjusted to pH of 7.4 at 37° C. and filtered. Freshly isolated cells were seeded into 96-well seahorse cell culture plate pre-coated for 3 h with poly-lysine. The cells were then cultured with 180 μl of the adjusted medium and the plate was centrifuged at 300 g for 5 min. The cells were then cultured in 37° C. non-CO₂ incubator for 1 h before the measurement. Mito-stress assay was performed according to the manufacturer protocol (Agilent technologies). The concentration of drugs used in the assay was 2 μM oligomycin, 4 μM FCCP, and 1 μM of rotenone/Antimycin A (sigma). For CMP and GMP 80,000 cells were used. For MPP 60,000 cells were used. For seahorse analysis of NR experiment (FIG. 7F) freshly FACS isolated Sorted CMPs (Lin⁻c-Kit⁺Sca1⁻CD34⁺FcγR^{-/low}) from young and old mice were pretreated with 1 mM NR or the 3 inhibitors (6 μM each) or DMSO for 12 h. Cells from NR treatment and DMSO were then exposed to the 31 or DMSO for 12h. The cells were cultured in in SFEM medium (STEM CELL Technologies, 09650) supplemented with 50 ng/mL mSCF (Peprotech, 250-03), 50 ng/mL mTPO (Peprotech, 315-14), 100 units/mL penicillin, 100 μg/mL streptomycin (Gibco, 15140-122). At the day of performing the assay the cells were changed to the readjusted medium as described above.

NanoString Analysis

Measuring of RNA expression by NanoString was done according to our previous publication (Chen et al., 2019) and according to manufacturer protocol (NanoString technologies). In brief, 1×10⁴ cells were lysed in 2 μL of lysis/binding solution (Applied Biosystems, 8500G14). The cell lysate was then used for hybridization reaction as following: 2 μL of cell lysate was mixed with 5 μL of nCounter hybridization buffer (NanoString), 2 μL of Core Tagset, 2 μL of extension Tagset, 0.5 μL of 0.6 nm Probe A working pool, 0.5 μL of 0.6 nm probe A extension Pool, 0.5 μL of 3 nm Probe B working pool, and 0.5 μL of 3 nM Probe B extension pool (from IDT technologies). 2 μL of Nuclease-free water was added to each reaction to reach a final volume of 15 μL. The reaction mixture was prepared in Strip tubes (from NanoString technologies). Then it was incubated at 67° C. using thermal cycler (Masterycler from Eppendorf) for 16 h. Afterward, the Nanostring chemistry was processed automatically using nCounter prep-station 5s (NanoString technologies) according to manufacturer protocol. Directly after the run, the nCounter Cartridge was loaded into nCounter digital analyzer 5s (NanoString technologies). For NanoString analysis on young and old mice exposed to DR or AL food, freshly FACS-Sorted MPPs (CD34⁺Flt3⁻ LSK) were used. For NR treatment, freshly FACS-Sorted MPPs (CD34⁺Flt3⁻ LSK) from young and old mice were pretreated with 1 mM NR or the 31 (6 μM each) or DMSO for 12 h. Cells from NR treatment and DMSO were divided into 2 halves and further treated with 3I (6 μM each) or DMSO as control. The cells were harvested for NanoString analysis as described above. Data analysis was done after background correction using nSolver advanced analysis software (v.4) and (R software v 3.3.2.). The following housekeeping genes were used for normalization: ActB, B2M, Gapdh, Gusb, Hprt, PGK1, Polr1b, Polr2a, Ppia, Rpl19, Sdha, and Tbp. Heatmaps and volcano plots was done using GraphPad prism software version 8.

Proteomics of CMP and GMPs

1×10⁵ PBS—washed cells (CMPs/GMPs) were FACS sorted directly into concentrated lysis buffer; the final composition was 1% SDS, 100 mM HEPES, 50 mM DTT, pH8.0). Samples were snap-frozen in dry ice before preparation for the proteomics data acquisition. After thawing on ice, samples were sonicated using a Bioruptor (60 sec ON/30 sec OFF, 10 cycles, high intensity at 20° C.) (Diagenode, Beligum), then heated to 95° C. for 10 min, before a repeat set of sonication cycles as before. Alklyation to block cysteines was carried out with iodoacetamide (15 mM final concentration, 30 min, dark, room temperature). Protein precipitation was carried out using 8× sample volume of ice-cold acetone and samples were left at −20° C. overnight. The following day, samples were centrifuged (20800× g, 30 min, 4° C.), supernatant carefully removed, and protein pellets washed twice with ice cold 80% acetone/20% water (300 μL), 10 min centrifugation (as above)). After removal of the second wash, pellets were air-dried before resuspension in the digestion buffer (1M Guanadine HCl in 0.1M HEPES, pH 8; LysC (1:100 enzyme:protein ratio), then incubated for 4 h at 37° C. The samples were diluted 1:1 with Milli-Q water (to reach 0.5 M GuaHCl) and incubated with trypsin (1:100 enzyme:protein) for 16 h at 37° C. The digests were then acidified with 10% trifluoroacetic acid and then desalted with Waters Oasis® HLB pElution Plate 30 μm (Waters Corporation, Milford, Mass., USA) in the presence of a slow vacuum. In this process, the columns were conditioned with 3×100 μL solvent B (80% acetonitrile; 0.05% formic acid) and equilibrated with 3×100 μL solvent A (0.05% formic acid in Milli-Q water). The samples were loaded, washed 3 times with 100 μL solvent A, and then eluted into PCR tubes with 50 μL solvent B. The eluates were dried down with the speed vacuum centrifuge prior to resuspension in 10 μL MS Buffer (5% acetonitrile, 95% Milli-Q water, with 0.1% formic acid) containing 0.25 μL of iRT kit (Biognosys AG) for the MS analyses.

LC-MS/MS (DDA for Library and DIA)

Data were acquired on a QE-HFX MS (Thermo), connected to an M-Class NanoAcquity (Waters).

The outlet of the analytical column was coupled directly to the mass spectrometer using the Proxeon nanospray source. Solvent A was water, 0.1% formic acid and solvent B was acetonitrile, 0.1% formic acid. Approximately 1 μg of each of the samples (reconstituted at estimated 1 μg/μL and spiked with iRT kit peptides (Biognosys AG, Switzerland)) were injected for LC-MS with a constant flow of solvent A, at 5 μL/min, in trapping mode. The trapping time was 6 min. Peptides were eluted via the analytical column with a constant flow of 0.3 μL/min. During the elution step, the percentage of solvent B increased in a non-linear fashion from 0% to 40% in 120 min. Total runtime was 145 min, including clean-up and column re-equilibration. The peptides were introduced into the mass spectrometer via a Pico-Tip Emitter 360 μm OD×20 μm ID; 10 μm tip (New Objective) and a spray voltage of 2.2 kV was applied. The capillary temperature was set at 300° C. The ion funnel RF was set to 40%. DDA data were acquired on pools of each condition, with the following settings: Full scan MS spectra with mass range 350-1650 m/z were acquired in profile mode in the Orbitrap with resolution of 120000. MS1 fill time was 20 ms, AGC target 3e6. Top N was used (=15) and the intensity threshold was 4e4. Normalized Collision Energy (NCE) with HCD was set to 27% and a 1.6 Da window was used for quadrupole isolation. MS2 data were acquired in profile mode from 200-2000 m/z. MS2 fill time was 25 ms or an AGC target of 2e5. Only 2-5+ charge states were selected for MS/MS. Dynamic exclusion was 20 s, and the peptide match “preferred” option was selected. For the DIA data, LC conditions remained unchanged. Full scan MS spectra with mass range 350-1650 m/z were acquired in profile mode in the Orbitrap with resolution of 120000. The default charge state was set to 3+. The filling time was set at maximum of 60 ms with limitation of 3e6 ions. DIA scans were acquired with 34 mass window segments of differing widths across the MS1 mass range. HCD fragmentation (stepped normalized collision energy; 25.5, 27, 30%) was applied and MS/MS spectra were acquired with a resolution of 30000 with a fixed first mass of 200 m/z after accumulation of 3e6 ions or after filling time of 40 ms (whichever occurred first). Data were acquired in profile mode. For data acquisition and processing of the raw data Xcalibur 4.0 (Thermo Scientific) was used in parallel with Tune version 2.9.

Data Analysis for DIA Data

For library creation, the DDA and DIA data were searched using the Pulsar search engine within Spectronaut (Spectronaut (version 13.1.190621.4365); Biognosys AG, Zurich, Switzerland). Data were searched against a species specific (Mus musculus) Swissprot database (01/2016, 16756 entries) alongside the database of common contaminants. The data were searched with trypsin/P specificity and the following modifications: Carbamidomethyl (C) (Fixed) and Oxidation (M)/Acetyl (Protein N-term) (Variable). A maximum of 2 missed cleavages was allowed. The identifications satisfied an FDR of 1% on both peptide and protein level. All other settings were the defaults from Biognosys (Bruderer et al., 2017; Rosenberger et al., 2017; Storey, 2002).

The resulting library contained 77363 precursors, corresponding to 4957 protein groups using Spectronaut protein inference. DIA data were then uploaded and searched against this spectral library in Spectronaut. Relative quantification was performed in the software for each pairwise comparison using the replicates from each condition. Quantification settings were modified from the defaults in the following way: Median and no Top N for the major and minor group quantities; data filtering on q-value percentile (0.5), no imputation, local cross-run normalization on q-value (sparse). The post-analysis settings were set to use the unpaired strategy. 7 samples were removed from the dataset due to poor quality recovery of material as seen in the MS chromatograms. The data (candidate table) and data reports were then exported as tables and further data analysis and visualization were performed with R-studio (version 1.0.153, employing R-version 3.6.1 (The R Foundation for Statistical Computing)) using in-house pipelines and scripts. Gene Set Enrichment Analysis (GSEA) on relevant pairwise comparisons was performed using Webgestalt ((Liao et al., 2019; Wang et al., 2013; Wang et al., 2017; Zhang et al., 2005).

Measurement of ATP, Lactate, and Pyruvate

The FACS purified HSC pellet (1000-1500 cells) lysed by 20 μL 1% NP40 on ice for 10 min and centrifuged at 8000 g for 5 min. The cell lysate was transferred into new clean tubes. The cellular ATP in low number of HSCs was measured by the ATP determination Kit (Thermo, A22066). Briefly, the ATP reaction solution and standard samples were prepared following the instruction manual with the reagents provided in the kit. The cell lysate (10 μL per sample) and standards were mixed with 100 μL ATP reaction solution and then added into the black 96-well flat bottom plate (Thermo). The plate was gently shaken, and the intensity of luminescence was measured for each well by microplate reader (Tecan). The relative cellular ATP level of each sample was calculated by subtracting the intensity value of empty control and divided by the initial cell number of the individual sample. For lactate or pyruvate measurement, the reagent was prepared according to the instruction manual of the Lactate Assay Kit (Sigma, MAK064) or Pyruvate Assay Kit (Sigma, MAK071). The cell lysate (3-5 μL per sample) was mixed with 50 μL Master Reaction Mix and added into the black 96-well flat bottom plate (Thermo). The plate was shaken briefly and incubated at room temperature for 10-30 min before the measurement. The cellular level of lactate or pyruvate was determined by measuring fluorescence intensity (λₑₓ=535 nm/λₑₘ=587 nm) on microplate reader (Tecan).

Immunofluorescence Staining

FACS purified cells were dropped on poly-Lysine pre-coated slides and seated at 4° C. for 0.5-1 h to let the cells attached to the slides. Then the cells were fixed by 3.7% paraformaldehyde/PBS for 10 min at room temperature, followed by washing with PBS. Slides were permeabilized in 0.3% Triton X-100/2% BSA/PBS for 30 mins at room temperature and blocked in 2% BSA/PBS for 30 mins at room temperature. The cells were then incubated with LC3 (Biorbyt, orb97657) and Tom20 (Santa Cruz Biotechnology, sc-11415) antibodies for 1 h at room temperature. After washing with PBS 3 times, the cells were then incubated with secondary antibodies: AF594 conjugated donkey anti-rabbit (Life Technologies, A21207) and AF647 conjugated donkey anti-mouse (Life Technologies, A31571) for 30 min at room temperature. After staining, the slides were washed by PBS for 3 times and mounted by mounting medium (Vector Laboratories, H-1000) containing 1 μg/mL DAPI. The cells were imaged on Zeiss Axio Imager Microscope (100× objective), and the images were processed using ZEN Software (Zeiss). The fluorescence intensity per cell was measured by ImageJ on 19 cells per mouse to calculate the mean fluorescence intensity (MFI) of an individual mouse.

LC-MS (Metabolites)

Different populations of hematopoietic stem and progenitor cells (30000 cells per condition) were purified by FACS. The cell pellets were resuspended with ice cold 80% methanol and immediately stored in −80° C. for overnight. The samples were thawed at 4° C. on the next day, and centrifuged at the highest speed for 20 min at 4° C. The supernatant was transferred into new tubes and mixed with equal volume of acetonitrile and stored at −80° C. until analysis. Liquid Chromatography coupled to Mass Spectroscopy (LC-MS) analysis was conducted on a Q-Exactive—Orbitrap mass spectrometer interfaced with a Dionex UltiMate 3000 LC system (Thermo). LC separation was conducted using a ZIC-cHILIC column (10×2.1 mm, 3 μm, Merck, USA), using a gradient from buffer B (10 mM CH₃COONH₄ pH5.8, 90% acetonitrile) to buffer A (10 mM CH₃COONH₄ pH5.8, 10% acetonitrile). The column compartment was kept at 20° C. during analysis. The maximum injection volume was 10 μL. The following parameters for the HESI ions source were used: Sheath gas flow rate—48 units; Aux gas flow rate—11 units; Sweep gas flow rate—2 units; Spray voltage—3.5 kV; Capillary temperature—256° C.; S-lens RF level—90 units; Aux gas heater temperature—413° C. Mass spectra were recorded by tSIM at positive polarity and maximum resolution (140,000). Metabolites in the samples were quantified by peak integration in the XCalibur software using processing setup. A cell extract containing nucleotides with fully C13-saturate ribose was generated by growing 293 cells in presence of 6×C13-labeled glucose. Samples were spiked with this cell extract, and concentrations of the endogenous metabolites were determined by comparing the peak area of the endogenous metabolite to the internal standard. Fragmentation analysis was performed to confirm identification of the detected metabolites.

Statistical Analysis and Generation of Graphs

All data are presented as mean with standard deviation (S.D.), except where stated in the figure legends. The numbers of biological replicates are stated in the figure legends. No statistical method was used to predetermine sample size. All data were tested for the normal distribution using the Shapiro-Wilk normality test (p<0.05). For data that passed normality test, we used parametric tests. To compare the significance between 2 groups with single condition or treatment, we used a two-tailed, unpaired t-test with Welch's correction. To compare the statistical significance of differences between two treatments or conditions, two-way ANOVA with Tukey's multiple comparison tests were used. For data that are not normally distributed, the Mann-Whitney Test was used. The significance level was set at 0.05 (5%). Statistical tests and generation of graphs were done using GraphPad prism v.8. Analysis of FACS data was done using Flowjo version 10. Seahorse data were analyzed using Wave software.

### TABLES OF THE EXAMPLES

TABLE 1
List of proteins from Sirtuin-signaling from Ingenuity Pathway Analysis (IPA) that was
carried out on proteome changes of GMPs from old (24 months, n = 5) compared to young
(3 months, n = 5) mice exposed to 2 weeks DR (red histogram of log2 fold changes in FIG. 12C).
The graph shows the top-10 enriched pathways using a cut-off including all significantly,
differently expressed proteins (q-value ≤ 0.05).

| | | | Expr False |
|---|---|---|---|
| | | | Discovery |
| | | | Rate |
| Symbol | Entrez Gene Name | Expr Log Ratio | (q-value) |
| ACLY | ATP citrate lyase | 0.017 | 3.59E−02 |
| ADAM 10 | ADAM metallopeptidase domain 10 | −0.007 | 3.87E−02 |
| AKT1 | AKT serinethreonine kinase 1 | 0.026 | 4.65E−02 |
| APEX1 | apurinicapyrimidinic | −0.074 | 1.04E−02 |
| | endodeoxyribonuclease 1 | | |
| ATG3 | autophagy related 3 | −0.072 | 2.01E−03 |
| ATG5 | autophagy related 5 | −0.107 | 1.35E−02 |
| ATG12 | autophagy related 12 | −1.868 | 4.06E−02 |
| ATG16L1 | autophagy related 16 like 1 | −0.440 | 8.94E−03 |
| ATP5F1C | ATP synthase F1 subunit gamma | 0.009 | 1.23E−02 |
| ATP5PB | ATP synthase peripheral stalk- | 0.094 | 3.65E−03 |
| | membrane subunit b | | |
| CPT1A | carnitine palmitoyltransferase 1A | 0.046 | 2.48E−10 |
| CYC1 | cytochrome c1 | 0.573 | 4.81E−04 |
| FOXO3 | forkhead box O3 | −0.822 | 1.03E−03 |
| G6PD | glucose-6-phosphate | −0.240 | 3.44E−02 |
| | dehydrogenase | | |
| GABARAPL1 | GABA type A receptor associated | −0.733 | 7.19E−04 |
| | protein like 1 | | |
| GABARAPL2 | GABA type A receptor associated | 0.255 | 3.75E−05 |
| | protein like 2 | | |
| GABPA | GA binding protein transcription | −0.060 | 2.14E−02 |
| | factor subunit alpha | | |
| GABPB1 | GA binding protein transcription | 0.301 | 7.31E−03 |
| | factor subunit beta 1 | | |
| GOT2 | glutamic-oxaloacetic transaminase 2 | −0.008 | 3.18E−02 |
| GSK3B | glycogen synthase kinase 3 beta | −0.140 | 2.96E−03 |
| GTF3C2 | general transcription factor IIIC | −0.294 | 1.25E−02 |
| | subunit 2 | | |
| H1F0 | H1 histone family member 0 | 0.155 | 6.91E−03 |
| HIST1H1C | histone cluster 1 H1 family member c | 0.114 | 1.99E−03 |
| HIST1H1D | histone cluster 1 H1 family member d | 0.237 | 3.34E−02 |
| LDHA | lactate dehydrogenase A | −0.251 | 8.43E−04 |
| MAP1LC3B | microtubule associated protein 1 | 0.307 | 1.95E−03 |
| | light chain 3 beta | | |
| MYCN | MYCN proto-oncogene, bHLH | −0.309 | 1.03E−02 |
| | transcription factor | | |
| NBN | nibrin | 0.060 | 3.50E−02 |
| NDUFA2 | NADH:ubiquinone oxidoreductase | 0.207 | 3.86E−03 |
| | subunit A2 | | |
| NDUFA4 | NDUFA4 mitochondrial complex | 0.000 | 3.51E−04 |
| | associated | | |
| NDUFA5 | NADH:ubiquinone oxidoreductase | −0.172 | 2.60E−03 |
| | subunit A5 | | |
| NDUFA9 | NADH:ubiquinone oxidoreductase | −0.070 | 7.38E−03 |
| | subunit A9 | | |
| NDUFAF2 | NADH:ubiquinone oxidoreductase | 0.396 | 1.07E−02 |
| | complex assembly factor 2 | | |
| NDUFB5 | NADH:ubiquinone oxidoreductase | 0.184 | 2.27E−03 |
| | subunit B5 | | |
| NDUFB8 | NADH:ubiquinone oxidoreductase | 0.294 | 1.29E−02 |
| | subunit B8 | | |
| NDUFB10 | NADH:ubiquinone oxidoreductase | 0.440 | 3.01E−05 |
| | subunit B10 | | |
| NDUFS1 | NADH:ubiquinone oxidoreductase | 0.136 | 2.86E−02 |
| | core subunit S1 | | |
| NDUFS3 | NADH:ubiquinone oxidoreductase | 0.074 | 3.61E−02 |
| | core subunit S3 | | |
| NDUFS6 | NADH:ubiquinone oxidoreductase | 0.163 | 1.48E−02 |
| | subunit S6 | | |
| NDUFS8 | NADH:ubiquinone oxidoreductase | 0.009 | 2.64E−02 |
| | core subunit S8 | | |
| NDUFV1 | NADH:ubiquinone oxidoreductase | −0.082 | 4.04E−02 |
| | core subunit V1 | | |
| NDUFV3 | NADH:ubiquinone oxidoreductase | 0.061 | 3.50E−02 |
| | subunit V3 | | |
| NQO1 | NAD(P)H quinone dehydrogenase 1 | 0.155 | 1.46E−03 |
| PCK2 | phosphoenolpyruvate | −0.006 | 6.50E−03 |
| | carboxykinase 2, mitochondrial | | |
| PGAM1 | phosphoglycerate mutase 1 | −0.075 | 1.17E−02 |
| PGK1 | phosphoglycerate kinase 1 | −0.056 | 2.42E−04 |
| POLR1C | RNA polymerase I and III subunit C | −0.132 | 3.66E−02 |
| POLR1D | RNA polymerase I and III subunit D | 0.146 | 6.64E−04 |
| POLR1E | RNA polymerase I subunit E | −0.040 | 4.73E−02 |
| PPID | peptidylprolyl isomerase D | −0.046 | 1.02E−02 |
| PPIF | peptidylprolyl isomerase F | −0.236 | 9.46E−03 |
| RELA | RELA proto-oncogene, NF-kB | 0.148 | 3.51E−02 |
| | subunit | | |
| SDHA | succinate dehydrogenase complex | −0.082 | 1.56E−03 |
| | flavoprotein subunit A | | |
| SDHB | succinate dehydrogenase complex | 0.036 | 1.65E−02 |
| | iron sulfur subunit B | | |
| SF3A1 | splicing factor 3a subunit 1 | 0.029 | 8.98E−03 |
| SIRT7 | sirtuin 7 | 0.269 | 2.23E−02 |
| SMARCA5 | SWISNF related, matrix associated, | 0.044 | 8.95E−03 |
| | actin dependent regulator of | | |
| | chromatin, subfamily a, member 5 | | |
| SOD1 | superoxide dismutase 1 | 0.041 | 2.82E−03 |
| SOD2 | superoxide dismutase 2 | 0.209 | 4.77E−03 |
| STAT3 | signal transducer and activator of | 0.007 | 5.58E−03 |
| | transcription 3 | | |
| TIMM9 | translocase of inner mitochondrial | 0.091 | 2.26E−05 |
| | membrane 9 | | |
| TIMM44 | translocase of inner mitochondrial | −0.015 | 4.35E−02 |
| | membrane 44 | | |
| TIMM50 | translocase of inner mitochondrial | 0.058 | 5.64E−03 |
| | membrane 50 | | |
| TIMM17B | translocase of inner mitochondrial | 0.113 | 4.23E−02 |
| | membrane 17B | | |
| TIMM8A | translocase of inner mitochondrial | 0.051 | 3.51E−03 |
| | membrane 8A | | |
| TOMM22 | translocase of outer mitochondrial | 0.223 | 1.14E−02 |
| | membrane 22 | | |
| TOMM70 | translocase of outer mitochondrial | 0.120 | 1.28E−02 |
| | membrane 70 | | |
| TRIM28 | tripartite motif containing 28 | 0.148 | 2.62E−02 |
| TUBA1C | tubulin alpha 1c | −0.211 | 1.15E−03 |
| UQCRC2 | ubiquinol-cytochrome c reductase | −0.040 | 1.36E−02 |
| | core protein 2 | | |
| WRN | Werner syndrome RecQ like | −0.355 | 1.61E−02 |
| | helicase | | |
| XPA | XPA, DNA damage recognition and | −0.180 | 5.84E−03 |
| | repair factor | | |
| XPC | XPC complex subunit, DNA | 0.016 | 9.63E−03 |
| | damage recognition and repair | | |
| | factor | | |
| XRCC5 | X-ray repair cross complementing 5 | 0.387 | 2.69E−05 |

TABLE 2
List of differentially regulated genes from FIG. 12G: 3I-exposed MPP
from young mice (Y.Con + 3I) versus 3I-exposed MPPs from old
mice (O.Con + 3I)

| m.RNA | Log2.FC | P-Values |
|---|---|---|
| Smc1-mRNA | 5.49 | 0.000166 |
| U2AF2-mRNA | 5.04 | 0.000166 |
| eIF4E-mRNA | 5.45 | 0.000166 |
| Eif3f-mRNA | 6.98 | 0.000458 |
| Eif4a1-mRNA | 5.24 | 0.000458 |
| U2AF1-mRNA | 5.74 | 0.000916 |
| SF3A2-mRNA | 4.07 | 0.00198 |
| Wapl-mRNA | 4.16 | 0.00198 |
| Nfatc1-mRNA | −1.5 | 0.00198 |
| Ldb1-mRNA | −2.09 | 0.00198 |
| SF3B2-mRNA | 4.62 | 0.00198 |
| Fli1-mRNA | −1.97 | 0.00326 |
| Erg-mRNA | −2.35 | 0.00326 |
| mfn2-mRNA | −1.63 | 0.00326 |
| Esco2-mRNA | −2.5 | 0.00398 |
| Eef2-mRNA | 7.39 | 0.00398 |
| Eif5a-mRNA | 7.22 | 0.00398 |
| SRSF9-mRNA | 4.84 | 0.00531 |
| SRSF3-mRNA | 6.76 | 0.00562 |
| Eif3h-mRNA | 5.58 | 0.00602 |
| Eif4a2-mRNA | 1.63 | 0.00602 |
| SRSF6-mRNA | 5.51 | 0.00646 |
| Lkb1-mRNA | −1.18 | 0.00646 |
| SF3B3-mRNA | 3.84 | 0.0068 |
| PP2A-mRNA | 3.19 | 0.00705 |
| TLR 3-mRNA | −8.13 | 0.00756 |
| 4E-BP1-mRNA | −2.12 | 0.00829 |
| IGF-1r-mRNA | −2.88 | 0.00875 |
| SF3B1-mRNA | 5.37 | 0.0107 |
| TRAIL (aka Apo2 ligand)-mRNA | −5.37 | 0.0107 |
| hsp60-mRNA | 3.34 | 0.0135 |
| Runx1-mRNA | −2.03 | 0.0141 |
| SRSF2-mRNA | 2.46 | 0.0143 |
| NFAT-mRNA | −3.01 | 0.0143 |
| Ldh2-mRNA | −3.57 | 0.0143 |
| p57-mRNA | −4.98 | 0.0143 |
| Eif4h-mRNA | 3.98 | 0.0143 |
| cMyc-mRNA | −1.2 | 0.0146 |
| Pds5B-mRNA | 3.97 | 0.0147 |
| Total XBP1-mRNA | −1.42 | 0.0154 |
| Eef1d-mRNA | 5.21 | 0.0154 |
| Mecom(Evi1)-mRNA | −3.39 | 0.0161 |
| Eef1g-mRNA | 2.87 | 0.0162 |
| Eif2a-mRNA | 3.53 | 0.0173 |
| Gata2-mRNA | −2.45 | 0.0185 |
| mfn1-mRNA | −1.33 | 0.0199 |
| SRSF7-mRNA | 2.36 | 0.0199 |
| Nfatc3-mRNA | −1.3 | 0.0212 |
| Smc3-mRNA | 3.76 | 0.022 |
| Scl/tal1-mRNA | −1.86 | 0.0225 |
| ERDJ4-mRNA | −2.96 | 0.0225 |
| RORc-mRNA | −4.98 | 0.0244 |
| Rad21-mRNA | 1.73 | 0.0301 |
| Pds5A-mRNA | −2.9 | 0.0307 |
| NIPBL-mRNA | 3.05 | 0.0308 |
| SRSF5-mRNA | 1.67 | 0.0308 |
| Rab 22-mRNA | −1.5 | 0.0308 |
| Csf3r(G-CSFR)-mRNA | −4.54 | 0.0329 |
| Egr1-mRNA | −3.15 | 0.0345 |
| Eif3d-mRNA | 1.53 | 0.0419 |
| TGF-b-mRNA | 1.13 | 0.0429 |
| ATP2A3-mRNA | −1.66 | 0.0429 |
| naprt-mRNA | −3.57 | 0.0429 |
| Pbx1-mRNA | −2.08 | 0.0429 |
| IDH1-mRNA | −1.72 | 0.0429 |
| SRSF10-mRNA | 2.41 | 0.0433 |
| drp1-mRNA | −1.14 | 0.0449 |
| GRP94-mRNA | 0.461 | 0.0455 |
| Sgo2a-mRNA | −2.49 | 0.0473 |

TABLE 3
List of differentially regulated genes from FIG. 12H: 3I-exposed MPPs
from young mice (Y.Con + 3I) versus 3I-exposed MPPs from old mice
that were pre-treated for 12 h with NR (O.NR + 3I).

| mRNA | LogZFC | P-Values |
|---|---|---|
| atp5a1-mRNA | 1.11 | 0.00456 |
| Myh9-mRNA | −2.45 | 0.0178 |
| unspliced XBP1-mRNA | 2.56 | 0.0178 |
| IDH1-mRNA | −2.62 | 0.0224 |
| Pds5A-mRNA | −3.08 | 0.0224 |
| Ino1-mRNA | −1.78 | 0.0283 |
| Pkm-mRNA | 1.73 | 0.0283 |
| NRas-mRNA | −2.16 | 0.0403 |

### REFERENCES

Akbari, M., Kirkwood, T. B. L., and Bohr, V. A. (2019). Mitochondria in the signaling pathways that control longevity and health span. Ageing Research Reviews 54, 100940.
Balaban, R. S., Nemoto, S., and Finkel, T. (2005). Mitochondria, Oxidants, and Aging. Cell 120, 483-495.
Barzilai, N., Crandall, J. P., Kritchevsky, S. B., and Espeland, M. A. (2016). Metformin as a Tool to Target Aging. Cell metabolism 23, 1060-1065.
Benedetto, A., and Gems, D. (2019). Autophagy promotes visceral aging in wild-type C. elegans. Autophagy 15, 731-732.
Bishop, N. A., and Guarente, L. (2007). Two neurons mediate diet-restriction-induced longevity in C. elegans. Nature 447, 545-549.
Bonora, M., Patergnani, S., Rimessi, A., De Marchi, E., Suski, J. M., Bononi, A., Giorgi, C., Marchi, S., Missiroli, S., Poletti, F., et al. (2012). ATP synthesis and storage. Purinergic Signal 8, 343-357.
Brown, K., Xie, S., Qiu, X., Mohrin, M., Shin, J., Liu, Y., Zhang, D., Scadden, David T., and Chen, D. (2013). SIRT3 Reverses Aging-Associated Degeneration. Cell Reports 3, 319-327.
Bruderer, R., Bernhardt, O. M., Gandhi, T., Xuan, Y., Sondermann, J., Schmidt, M., Gomez-Varela, D., and Reiter, L. (2017). Optimization of Experimental Parameters in Data-Independent Mass Spectrometry Significantly Increases Depth and Reproducibility of Results. Molecular &amp; Cellular Proteomics 16, 2296-2309.
Cantó, C., Menzies, Keir J., and Auwerx, J. (2015). NAD+ Metabolism and the Control of Energy Homeostasis: A Balancing Act between Mitochondria and the Nucleus. Cell Metabolism 22, 31-53.
Carter, C. S., Ramsey, M. M., and Sonntag, W. E. (2002). A critical analysis of the role of growth hormone and IGF-1 in aging and lifespan. Trends in Genetics 18, 295-301.
Chen, Z., Amro, E. M., Becker, F., Holzer, M., Rasa, S. M. M., Njeru, S. N., Han, B., Di Sanzo, S., Chen, Y., Tang, D., et al. (2019). Cohesin-mediated NF-κB signaling limits hematopoietic stem cell self-renewal in aging and inflammation. The Journal of Experimental Medicine 216, 152-175.
Collino, S., Martin, F.-P. J., Montoliu, I., Barger, J. L., Da Silva, L., Prolla, T. A., Weindruch, R., and Kochhar, S. (2013). Transcriptomics and Metabonomics Identify Essential Metabolic Signatures in Calorie Restriction (CR) Regulation across Multiple Mouse Strains. Metabolites 3, 881-911.
Deutsch, E. W., Csordas, A., Sun, Z., Jarnuczak, A., Perez-Riverol, Y., Ternent, T., Campbell, D. S., Bernal-Llinares, M., Okuda, S., Kawano, S., et al. (2016). The ProteomeXchange consortium in 2017: supporting the cultural change in proteomics public data deposition. Nucleic Acids Research 45, D1100-D1106.
Dunn, S. E., Kari, F. W., French, J., Leininger, J. R., Travlos, G., Wilson, R., and Barrett, J. C. (1997). Dietary Restriction Reduces Insulin-like Growth Factor I Levels, Which Modulates Apoptosis, Cell Proliferation, and Tumor Progression in p53-deficient Mice. Cancer Research 57, 4667-4672.
Fontana, L., Partridge, L., and Longo, V. D. (2010). Extending Healthy Life Span—From Yeast to Humans. Science 328, 321-326.
Geach, T. (2016). Accelerated ageing slowed by reduced calorie intake. Nature Reviews Endocrinology 12, 623.
Grandison, R. C., Piper, M. D. W., and Partridge, L. (2009). Amino-acid imbalance explains extension of lifespan by dietary restriction in Drosophila. Nature 462, 1061-1064.
Green, D. R., Galluzzi, L., and Kroemer, G. (2011). Mitochondria and the Autophagy-Inflammation-Cell Death Axis in Organismal Aging. Science 333, 1109-1112.
Guarente, L. (2008). Mitochondria—A Nexus for Aging, Calorie Restriction, and Sirtuins? Cell 132, 171-176.
Haas, R. H. (2019). Mitochondrial Dysfunction in Aging and Diseases of Aging. Biology (Basel) 8, 48.
Hahn, O., Drews, L. F., Nguyen, A., Tatsuta, T., Gkioni, L., Hendrich, O., Zhang, Q., Langer, T., Pletcher, S., Wakelam, M. J. O., et al. (2019). A nutritional memory impairs survival, transcriptional and metabolic response to dietary restriction in old mice. bioRxiv, 730853.
Hansen, M., Chandra, A., Mitic, L. L., Onken, B., Driscoll, M., and Kenyon, C. (2008). A Role for Autophagy in the Extension of Lifespan by Dietary Restriction in C. elegans. PLOS Genetics 4, e24.
Ho, T. T., Warr, M. R., Adelman, E. R., Lansinger, O. M., Flach, J., Verovskaya, E. V., Figueroa, M. E., and Passegué, E. (2017). Autophagy maintains the metabolism and function of young and old stem cells. Nature 543, 205-210.
Holt, P. R., Moss, S. F., Heydari, A. R., and Richardson, A. (1998). Diet Restriction Increases Apoptosis in the Gut of Aging Rats. The Journals of Gerontology: Series A 53A, B168-B172.
Ito, K., Carracedo, A., Weiss, D., Arai, F., Ala, U., Avigan, D. E., Schafer, Z. T., Evans, R. M., Suda, T., Lee, C.-H., et al. (2012). A PML-PPAR-δ pathway for fatty acid oxidation regulates hematopoietic stem cell maintenance. Nat Med 18, 1350-1358.
Jia, K., and Levine, B. (2007). Autophagy is Required for Dietary Restriction-Mediated Life Span Extension in C. elegans. Autophagy 3, 597-599.
Kapahi, P., Zid, B. M., Harper, T., Koslover, D., Sapin, V., and Benzer, S. (2004). Regulation of Lifespan in Drosophila by Modulation of Genes in the TOR Signaling Pathway. Current Biology 14, 885-890.
Lee, C.-K., Allison, D. B., Brand, J., Weindruch, R., and Prolla, T. A. (2002). Transcriptional profiles associated with aging and middle age-onset caloric restriction in mouse hearts. Proceedings of the National Academy of Sciences 99, 14988-14993.
Lee, C.-K., Klopp, R. G., Weindruch, R., and Prolla, T. A. (1999). Gene Expression Profile of Aging and Its Retardation by Caloric Restriction. Science 285, 1390-1393.
Lee, K. P., Simpson, S. J., Clissold, F. J., Brooks, R., Ballard, J. W. O., Taylor, P. W., Soran, N., and Raubenheimer, D. (2008). Lifespan and reproduction in <em>Drosophila</em>: New insights from nutritional geometry. Proceedings of the National Academy of Sciences 105, 2498-2503.
Lee, S.-J., Hwang, A. B., and Kenyon, C. (2010). Inhibition of Respiration Extends C. elegans Life Span via Reactive Oxygen Species that Increase HIF-1 Activity. Current Biology 20, 2131-2136.
Liao, Y., Wang, J., Jaehnig, E. J., Shi, Z., and Zhang, B. (2019). WebGestalt 2019: gene set analysis toolkit with revamped UIs and APIs. Nucleic Acids Research 47, W199-W205.
Lin, S.-J., Kaeberlein, M., Andalis, A. A., Sturtz, L. A., Defossez, P.-A., Culotta, V. C., Fink, G. R., and Guarente, L. (2002). Calorie restriction extends Saccharomyces cerevisiae lifespan by increasing respiration. Nature 418, 344-348.
Mair, W., Goymer, P., Pletcher, S. D., and Partridge, L. (2003). Demography of Dietary Restriction and Death in <em>Drosophila</em>. Science 301, 1731-1733.
Mariño, G., Ugalde, A. P., Fernãndez, Ã. F., Osorio, F. G., Fueyo, A., Freije, J. M. P., and López-Otín, C. (2010). Insulin-like growth factor 1 treatment extends longevity in a mouse model of human premature aging by restoring somatotroph axis function. Proceedings of the National Academy of Sciences 107, 16268-16273.
Mitchell, S. J., Madrigal-Matute, J., Scheibye-Knudsen, M., Fang, E., Aon, M., González-Reyes, J. A., Cortassa, S., Kaushik, S., Gonzalez-Freire, M., Patel, B., et al. (2016). Effects of Sex, Strain, and Energy Intake on Hallmarks of Aging in Mice. Cell metabolism 23, 1093-1112.
Mohrin, M., Shin, J., Liu, Y., Brown, K., Luo, H., Xi, Y., Haynes, C. M., and Chen, D. (2015). A mitochondrial UPR-mediated metabolic checkpoint regulates hematopoietic stem cell aging. Science 347, 1374-1377.
Morselli, E., Maiuri, M. C., Markaki, M., Megalou, E., Pasparaki, A., Palikaras, K., Criollo, A., Galluzzi, L., Malik, S. A., Vitale, I., et al. (2010). Caloric restriction and resveratrol promote longevity through the Sirtuin-1-dependent induction of autophagy. Cell Death & Disease 1, e10-e10.
Mouchiroud, L., Houtkooper, R. H., Moullan, N., Katsyuba, E., Ryu, D., Cantó, C., Mottis, A., Jo, Y.-S., Viswanathan, M., Schoonjans, K., et al. (2013). The NAD(+)/Sirtuin Pathway Modulates Longevity through Activation of Mitochondrial UPR and FOXO Signaling. Cell 154, 430-441.
Nisoli, E., Tonello, C., Cardile, A., Cozzi, V., Bracale, R., Tedesco, L., Falcone, S., Valerio, A., Cantoni, O., Clementi, E., et al. (2005). Calorie Restriction Promotes Mitochondrial Biogenesis by Inducing the Expression of eNOS. Science 310, 314-317.
Ochocki, J. D., and Simon, M. C. (2013). Nutrient-sensing pathways and metabolic regulation in stem cells. The Journal of Cell Biology 203, 23-33.
Ralser, M., Wamelink, M. M., Struys, E. A., Joppich, C., Krobitsch, S., Jakobs, C., and Lehrach, H. (2008). A catabolic block does not sufficiently explain how 2-deoxy-<span class=“sc”>d</span>-glucose inhibits cell growth. Proceedings of the National Academy of Sciences 105, 17807-17811.
Rosenberger, G., Bludau, I., Schmitt, U., Heusel, M., Hunter, C. L., Liu, Y., MacCoss, M. J., MacLean, B. X., Nesvizhskii, A. I., Pedrioli, P. G. A., et al. (2017). Statistical control of peptide and protein error rates in large-scale targeted data-independent acquisition analyses. Nature Methods 14, 921.
Satoh, A., Brace, C. S., Rensing, N., Cliften, P., Wozniak, D. F., Herzog, E. D., Yamada, K. A., and Imai, S I. (2013). Sirt1 extends life span and delays aging in mice through the regulation of Nk2 homeobox 1 in the DMH and LH. Cell metabolism 18, 416-430.
Snoeck, H.-W. (2017). Mitochondrial regulation of hematopoietic stem cells. Curr Opin Cell Biol 49, 91-98.
Storey, J. D. (2002). A direct approach to false discovery rates. Journal of the Royal Statistical Society: Series B (Statistical Methodology) 64, 479-498.
Sun, N., Youle, R. J., and Finkel, T. (2016). The Mitochondrial Basis of Aging. Mol Cell 61, 654-666.
Tang, D., Tao, S., Chen, Z., Koliesnik, I. O., Calmes, P. G., Hoerr, V., Han, B., Gebert, N., Zörnig, M., Löffler, B., et al. (2016). Dietary restriction improves repopulation but impairs lymphoid differentiation capacity of hematopoietic stem cells in early aging. The Journal of Experimental Medicine 213, 535-553.
Vannini, N., Campos, V., Girotra, M., Trachsel, V., Rojas-Sutterlin, S., Tratwal, J., Ragusa, S., Stefanidis, E., Ryu, D., Rainer, P. Y., et al. (2019). The NAD-Booster Nicotinamide Riboside Potently Stimulates Hematopoiesis through Increased Mitochondrial Clearance. Cell Stem Cell 24, 405-418.e407.
Vannini, N., Girotra, M., Naveiras, O., Nikitin, G., Campos, V., Giger, S., Roch, A., Auwerx, J., and Lutolf, M. P. (2016). Specification of haematopoietic stem cell fate via modulation of mitochondrial activity. Nature Communications 7, 13125.
Vermeij, W. P., Dollé, M. E. T., Reiling, E., Jaarsma, D., Payan-Gomez, C., Bombardieri, C. R., Wu, H., Roks, A. J. M., Botter, S. M., van der Eerden, B. C., et al. (2016). Restricted diet delays accelerated ageing and genomic stress in DNA-repair-deficient mice. Nature 537, 427-431.
Vizcaino, J. A., Csordas, A., del-Toro, N., Dianes, J. A., Griss, J., Lavidas, I., Mayer, G., Perez-Riverol, Y., Reisinger, F., Ternent, T., et al. (2015). 2016 update of the PRIDE database and its related tools. Nucleic Acids Research 44, D447-D456.
Volk, M. J., Pugh, T. D., Kim, M., Frith, C. H., Daynes, R. A., Ershler, W. B., and Weindruch, R. (1994). Dietary Restriction from Middle Age Attenuates Age-associated Lymphoma Development and Interleukin 6 Dysregulation in C57BL/6 Mice. Cancer Research 54, 3054-3061.
Walker, J. E. (2009). The NADH:ubiquinone oxidoreductase (complex I) of respiratory chains. Quarterly Reviews of Biophysics 25, 253-324.
Wang, J., Duncan, D., Shi, Z., and Zhang, B. (2013). WEB-based GEne SeT AnaLysis Toolkit (WebGestalt): update 2013. Nucleic Acids Research 41, W77-W83.
Wang, J., Vasaikar, S., Shi, Z., Greer, M., and Zhang, B. (2017). WebGestalt 2017: a more comprehensive, powerful, flexible and interactive gene set enrichment analysis toolkit. Nucleic Acids Research 45, W130-W137.
Wei, M., Fabrizio, P., Hu, J., Ge, H., Cheng, C., Li, L., and Longo, V. D. (2008). Life Span Extension by Calorie Restriction Depends on Rim15 and Transcription Factors Downstream of Ras/PKA, Tor, and Sch9. PLOS Genetics 4, el 3.
Weir, H. J., Yao, P., Huynh, F. K., Escoubas, C. C., Goncalves, R. L., Burkewitz, K., Laboy, R., Hirschey, M. D., and Mair, W. B. (2017). Dietary Restriction and AMPK Increase Lifespan via Mitochondrial Network and Peroxisome Remodeling. Cell Metabolism 26, 884-896.e885.
Wilhelm, T., Byrne, J., Medina, R., Kolundzid, E., Geisinger, J., Hajduskova, M., Tursun, B., and Richly, H. (2017). Neuronal inhibition of the autophagy nucleation complex extends life span in post-reproductive C. elegans. Genes & Development 31, 1561-1572.
Xiao, W., Wang, R.-S., Handy, D. E., and Loscalzo, J. (2017). NAD(H) and NADP(H) Redox Couples and Cellular Energy Metabolism. Antioxidants & Redox Signaling 28, 251-272.
Yamamoto, R., Wilkinson, A. C., Ooehara, J., Lan, X., Lai, C.-Y., Nakauchi, Y., Pritchard, J. K., and Nakauchi, H. (2018). Large-Scale Clonal Analysis Resolves Aging of the Mouse Hematopoietic Stem Cell Compartment. Cell Stem Cell 22, 600-607.e604.
Zarse, K., Schmeisser, S., Groth, M., Priebe, S., Beuster, G., Kuhlow, D., Guthke, R., Platzer, M., Kahn, C. R., and Ristow, M. (2012). Impaired Insulin/IGF1 Signaling Extends Life Span by Promoting Mitochondrial L-Proline Catabolism to Induce a Transient ROS Signal. Cell Metabolism 15, 451-465.
Zhang, B., Kirov, S., and Snoddy, J. (2005). WebGestalt: an integrated system for exploring gene sets in various biological contexts. Nucleic Acids Research 33, W741-W748.
Zhang, H., Ryu, D., Wu, Y., Gariani, K., Wang, X., Luan, P., D'Amico, D., Ropelle, E. R., Lutolf, M. P., Aebersold, R., et al. (2016). NAD<sup>+</sup>repletion improves mitochondrial and stem cell function and enhances life span in mice. Science 352, 1436-1443.
Zid, B. M., Rogers, A. N., Katewa, S. D., Vargas, M. A., Kolipinski, M. C., Lu, T. A., Benzer, S., and Kapahi, P. (2009). 4E-BP Extends Lifespan upon Dietary Restriction by Enhancing Mitochondrial Activity in Drosophila. Cell 139, 149-160.

1. A method of treating or reducing the risk of an age-related medical condition in a subject, having or at risk of an age-related medical condition, comprising: administrating a nicotinamide adenine dinucleotide (NAD) precursor to a subject, wherein said NAD precursor is administered in combination with a calorie restriction diet (CRD) and/or a calorie restriction mimetic (CRM).

2. The method according to claim 1, wherein the NAD precursor is nicotinamide riboside (NR).

3. The method according to claim 1, wherein the CRM is a fasting mimicking diet product.

4. The method according to claim 1, wherein the CRM is a chemical compound/drug, preferably selected from the group consisting of resveratrol, metformin, oxaloacetate, rimonabant, lipoic acid, 2-deoxy-D-glucose and rapamycin.

5. The method according to claim 1, wherein the age-related medical condition is associated with a decline in mitochondrial function.

6. The method according to claim 1, wherein the treatment and/or prevention of an age-related medical condition comprises slowing, reversing and/or inhibiting the ageing process.

7. The method according to claim 1, wherein the subject is human and is more than 40 years old.

8. The method according to claim 1, wherein the NAD precursor antagonizes an age-related decline of mitochondrial function.

9. The method according to claim 1, wherein the CRD and/or CRM promote metabolic plasticity, mitochondrial metabolism and/or metabolic stress responses.

10. A pharmaceutical combination, comprising
a nicotinamide adenine dinucleotide (NAD) precursor, and
a calorie restriction mimetic (CRM).

11. The pharmaceutical combination according to claim 10,
wherein the NAD precursor is nicotinamide riboside (NR).

12. The pharmaceutical combination according to claim 10, wherein the CRM is selected from the group consisting of resveratrol, metformin, oxaloacetate, rimonabant, lipoic acid, 2-deoxy-D-glucose and rapamycin.

13. The pharmaceutical combination according to claim 10, wherein
the NAD precursor is in a pharmaceutical composition in admixture with a pharmaceutically acceptable carrier, and the CRM compound is in a separate pharmaceutical composition in admixture with a pharmaceutically acceptable carrier, or
the NAD precursor and the CRM compound are present in a kit, in spatial proximity but in separate containers and/or compositions, or
the NAD precursor and the CRM compound are combined in a single pharmaceutical composition in admixture with a pharmaceutically acceptable carrier.

14. A method of treating or reducing the risk of an age-related medical condition in a subject having or at risk of an age-related medical condition, comprising administrating a pharmaceutical combination according to claim 10 to a subject.

15. The method according to claim 14, wherein the age-related medical condition is associated with a decline in mitochondrial function.

16. The method according to claim 8, wherein the NAD precursor enhances and/or restores a therapeutic response of a human subject to CRD and/or CRM administration.

17. The method according to claim 16, wherein the subject is over 40 years old.

18. The pharmaceutical combination according to claim 10, wherein the CRM is rapamycin and a fasting mimicking diet product.
